(19) United States
(12) Patent Application Publication
(10) Pub. No.: US 2009/0075832 A1
(43) Pub. Date: Mar. 19, 2009

(54) Compositions and Methods for Classifying Biological Samples

(75) Inventors: Toomas Neuman, San Diego, CA (US); Mehis Pold, San Diego, CA (US)

Correspondence Address: King Spalding LLP, 4 Embarcadero Center, Suite 3500, San Francisco, CA, 94111, US

(73) Assignee: CeMines, Inc, Golden, CO (US)

(21) Appl. No.: 11/817,010

(22) PCT Filed: Feb. 24, 2006
(86) PCT No.: PCT/US06/06431

§ 371 Date: Apr. 7, 2008

Related U.S. Application Data
(60) Provisional application No. 60/656,859, filed on Feb. 24, 2005.

(51) Int. Cl. C40B 30/04; C40B 40/10; G01N 33/53

(52) U.S. Cl. 506/9; 506/18; 436/501

(57) ABSTRACT

The present invention relates to autoantibodies and the detection thereof with peptide epitopes. The invention also relates to autoantibody patterns and their correlation with biological class distinctions.

### BACKGROUND

Cancer is the second leading cause of death in the United States. Despite focused research in conventional diagnostics and therapies, the five-year survival rate has improved only minimally in the past 25 years. Better understanding of the complexity of tumorigenesis is required for the development and commercialization of much-needed, efficacious diagnostic and therapeutic products.

Based on observed immune responses to human tumors, it has been suggested that serum autoantibodies (“aABs”) could be used in cancer diagnostics (Fernandez-Madrid et al., Clin Cancer Res. 5:1393-400 (1999)). For example, the presence of certain serum aABs can reportedly predict the manifestation of lung cancer among at-risk patients (Lubin et al., Nat Med. 1995; 1:701-2), as well as the prognosis for non-small cell lung cancer (NSCLC) patients (Blaes et al., Ann Thorac Surg. 2000; 69:254-8). Notably however, such cancer studies have only reported on a small number of markers that are not determinative of the presence or absence of cancer and have invariably focused on the appearance of cancer-related serum aABs and their tumor-associated antigens in cancer patients (Vernino et al., Clin. Cancer Res. 10:7270-5 (2004); Metcalfe et al., Breast Cancer Res. 2:438-43 (2000); Tan, J. Clin. Invest. 108:1411-5 (2001); Lubin et al., Nat Med. 1:701-2 (1995); Torchilin et al., Trends Immunol. 22:424-7 (2001); Koziol et al., Clin. Cancer Res. 9:5120-5126, (2003); Zhang et al., Clin. Exp. Immunol. 125:3-9, (2001)). Further, the low frequency with which an autoantibody specific for any individual tumor-associated antigen is detected has precluded the use of autoantibodies as useful diagnostic markers.

Few studies concerning the multiplex analysis of aABs in a disease condition have been reported. The pioneering study by Robinson et al. in this specific area was published in 2002 and described multiple aABs that recognized a variety of biomolecules and were present in eight distinct human autoimmune diseases, including systemic lupus erythematosus and rheumatoid arthritis (Robinson et al., Nat Med. 8:295-301 (2002)). No similar studies concerning cancer have been reported.

All currently used aAB detection strategies have their intrinsic strengths and weaknesses. For example, detection of an individual aAB by ELISA offers simplicity. The major weakness of this approach, however, is that it is silent with respect to other potentially informative aABs and therefore limited in its predictive value. The SEREX analysis (serological analysis of expression cDNA libraries) enables simultaneous identification of different aABs with known specificity (Gure et al., Cancer Res. 58:1034-41 (1998)). This technique, however, is time and labor consuming, and, thus, unsuitable for clinical use. Western blotting with patient sera quickly identifies the size of potential autoantigens in a protein sample but is restricted in its informative capacity by the protein samples used and the limited resolution of autoantibody:antigen complexes, and provides no further information regarding the identity of autoantigens (Fernandez-Madrid et al., Clin Cancer Res. 5:1393-400 (1999)).

In conclusion, autoantibody patterns determinative for cancer, cancer subtypes, and other aspects of the disease have not been described. Further, high-throughput analytical tools for detecting autoantibodies and autoantibody patterns in biological samples that are relevant to the diagnosis and characterization of cancer would be of great benefit.

### SUMMARY OF INVENTION

The present invention concerns the detection of autoantibodies (aABs) in biological samples, and exploits differences in immune status, as determined by autoantibody profiling, to distinguish physiological states or phenotypes (referred to herein as classes) and yield diagnostic and prognostic information. The present invention uses peptide epitopes to mimic antigen-antibody binding and determine autoantibody binding activities (autoantibody profiling) in biological samples as a semi-quantifiable measure of immune status. Methods for selecting sets of informative epitopes useful for autoantibody profiling and class prediction, including diagnostic and prognostic determinations, as well as sets of informative epitopes useful for particular disease class distinctions are provided. In one example, as disclosed herein, patients with different tumor status have detectable differences in their serum aAB profiles, which has diagnostic relevance. A set of synthetic peptides is used to measure autoantibody binding activities in cancer and non-cancer samples, and a subset of informative epitopes is identified and used to characterize the immune status associated with the cancer and provide a highly accurate cancer diagnostic. In another example disclosed herein, a set of informative epitopes useful for distinguishing lung cancer subclasses is provided. Advantageously, the invention uses autoantibody binding activity pattern recognition and sets of informative epitopes because combinations of multiple autoantibody binding activities as composites possess a greater potential to characterize cancer accurately compared with traditional single-entity biomarkers, including single aABs.

In addition to sets of informative epitopes that may be used to detect autoantibody binding activity patterns that are diagnostic for a variety of cancers, the present invention provides sets of informative epitopes that may be used to determine a specific disease stage or the histopathological phenotype of a tumor based on the autoantibody binding activity patterns detected therewith. Additionally provided herein are sets of informative epitopes that may be used to classify a sample as being from an individual at high risk for manifestation of a disease based on the autoantibody binding activity patterns detected therewith. Notably, unlike gene-arrays, the biological samples used for the aAB-tests disclosed herein do not require a biopsy or time-consuming sample purification.

Importantly, the present invention makes use of epitopes, rather than whole proteins or fragments thereof, to probe samples for autoantibodies. As demonstrated herein, epitopes corresponding to different segments of a single protein can exhibit discordant differences in their binding activities between samples from different classes. As a consequence, autoantibody detection with whole proteins or fragments thereof (i.e., composites of multiple epitopes) can be uninformative with respect to class distinction, while the use of individual epitopes within a single protein may be highly informative. For example, a first epitope may have an epitope binding activity present at a certain frequency in non-cancer samples, and lack detectable epitope binding activity in samples from small cell lung cancer patients. A second epitope, corresponding to the same protein and not overlapping with the first epitope, may have an abundant epitope binding activity present at a similar frequency in both normal samples and cancer samples. In this instance, the first epitope would be informative, as discussed herein, while the second epitope and the whole protein would not be informative to class distinction based on these results.

Another important aspect of the diagnostic and prognostic methods disclosed herein is that they take into consideration autoantibodies of varied distribution, notably including epitope binding activities that are present in normal samples and decreased in disease samples. That is, the present methods do not focus solely on autoantibodies that appear in disease conditions in response to the appearance of disease-associated autoantigens. Rather, the present invention utilizes a variety of epitopes, many of which detect high levels of epitope binding activities in normal samples at a certain frequency and reveal low or undetectable levels of epitope binding activities in samples corresponding to a disease condition. Despite the fact that autoantibodies capable of binding such epitopes are frequently not detectable in disease samples, these epitopes are, nonetheless, informative with respect to class distinction, and are useful in the diagnostic and prognostic methods disclosed herein.

Accordingly, in one aspect, the present invention provides methods of identifying a set of informative epitopes, the autoantibody binding activities of which correlate with a class distinction between samples. The methods comprise sorting epitopes by the degree to which their autoantibody binding activity in samples correlates with a class distinction, and determining whether the correlation is stronger than expected by chance. An epitope for which autoantibody binding activity correlates with a class distinction more strongly than expected by chance is an informative epitope. A set of informative epitopes is identified. In one embodiment, the class distinction is determined between known classes. Preferably, the class distinction is between a disease class and a non-disease class, more preferably a cancer class and a normal class. In another preferred embodiment, the class distinction is between a high risk class and a non-disease class, more preferably a high risk cancer class and a non-cancer class. A known class can also be a class of individuals who respond well to chemotherapy or a class of individuals who do not respond well to chemotherapy.

In another embodiment, the known class distinction is a disease class distinction, preferably a cancer class distinction, still more preferably a lung cancer class distinction, a breast cancer class distinction, a gastrointestinal cancer class distinction, or a prostate cancer class distinction. In one embodiment, the known class distinction is a lung cancer class distinction between an SCLC class and an NSCLC class.

Sorting epitopes by the degree to which their autoantibody binding activity in samples correlates with a class distinction and determining the significance of the correlation can be carried out by neighborhood analysis (e.g., employing a signal to noise routine, a Pearson correlation routine, or a Euclidean distance routine) that comprises defining an idealized autoantibody binding activity pattern, wherein the idealized pattern is autoantibody binding activity that is uniformly high in a first class and uniformly low in a second class; and determining whether there is a high density of epitopes for which autoantibody binding activity is similar to the idealized pattern, as compared to an equivalent random pattern. The signal to noise routine is:

P(g,c)=(μ₁(g)−μ₂(g))/(σ₁(g)+σ₂(g)),

wherein g is the autoantibody binding activity value for an epitope; c is the class distinction, μ₁(g) is the mean of the autoantibody binding activity values for g for the first class; μ₂(g) is the mean of the autoantibody binding activity values for g for the second class; σ₁(g) is the standard deviation for the first class; and σ₂(g) is the standard deviation for the second class.

In one embodiment, a signal to noise routine is used to determine a weighted vote for an informative epitope for the classification of cancer without neighborhood analysis.

Another aspect of the present invention is a method of assigning a sample to a known or putative class, comprising determining a weighted vote of one or more informative epitopes (e.g., greater than 20, 50, 100, 150) for one of the classes in accordance with a model built with a weighted voting scheme, wherein the magnitude of each vote depends on the autoantibody binding activity of the sample for the given epitope and on the degree of correlation of the autoantibody binding activity for the given epitope with class distinction; and summing the votes to determine the winning class. The weighted voting scheme is:

V_{g}=a_{g}(x_{g}−b_{g}),

wherein V_{g} is the weighted vote of the epitope, g; a_{g} is the correlation between autoantibody binding activity for the epitope and class distinction, P(g,c), as defined herein; b_{g}=(μ₁(g)+μ₂(g))/2 which is the average of the mean log₁₀ autoantibody binding activity value for the epitope in a first class and a second class; x_{g} is the log₁₀ autoantibody binding activity value for the epitope in the sample to be tested; and wherein a positive V value indicates a vote for the first class, and a negative V value indicates a negative vote for the first class (a vote for the second class). A prediction strength can also be determined, wherein the sample is assigned to the winning class if the prediction strength is greater than a particular threshold, e.g., 0.3. The prediction strength is determined by:

(V_{win}−Vₗₒₛₑ)/(V_{win}+Vₗₒₛₑ),

wherein V_{win} and Vₗₒₛₑ are the vote totals for the winning and losing classes, respectively.

The invention also encompasses a method of determining a weighted vote for an informative epitope to be used in classifying a sample, comprising determining a weighted vote for one of the classes for one or more informative epitopes, wherein the magnitude of each vote depends on the autoantibody binding activity of the sample for the epitope and on the degree of correlation of the autoantibody binding activity for the epitope with class distinction. The votes may be summed to determine the winning class.

Yet another embodiment of the present invention is a method for ascertaining a plurality of classifications from two or more samples, comprising clustering samples by autoantibody binding activities to produce putative classes; and determining whether the putative classes are valid by carrying out class prediction based on putative classes and assessing whether the class predictions have a high prediction strength. The clustering of the samples can be performed, for example, according to a self organizing map. The self organizing map is formed of a plurality of Nodes, N, and the map clusters the vectors according to a competitive learning routine. The competitive learning routine is:

fᵢ₊₁(N)=fᵢ(N)+τ(d(N,Nₚ),i)(P−fᵢ(N))

wherein i=number of iterations, N=the node of the self organizing map, τ=learning rate, P=the subject working vector, d=distance, Nₚ=node that is mapped nearest to P, and fᵢ(N) is the position of N at i. To determine whether the putative classes are valid the steps for building the weighted voting scheme can be carried out as described herein and class prediction may be performed on the samples.

The invention also pertains to a method for classifying a sample obtained from an individual into a class, comprising assessing the sample for autoantibody binding activity for at least one epitope; and, using a model built with a weighted voting scheme, classifying the sample as a function of autoantibody binding activity of the sample with respect to that of the model.

The present invention also pertains to a method, e.g., for use in a computer system, for classifying a sample obtained from an individual. The method comprises providing a model built by a weighted voting scheme; assessing the sample for autoantibody binding activity for at least one epitope, to thereby obtain an autoantibody binding activity value for each epitope; using the model built with a weighted voting scheme, classifying the sample comprising comparing the autoantibody binding activity of the sample to the model, to thereby obtain a classification; and providing an output indication of the classification. The routines for the weighted voting scheme and neighborhood analysis are described herein. The method can be carried out using a vector that represents a series of autoantibody binding activity values for the samples. The vectors are received by the computer system, and then subjected to the above steps. The methods further comprise performing cross-validation of the model. The cross-validation of the model involves eliminating or withholding a sample used to build the model; using a weighted voting routine, building a cross-validation model for classifying without the eliminated sample; and using the cross-validation model, classifying the eliminated sample into a winning class by comparing the autoantibody binding activity values of the eliminated sample to autoantibody binding activity values of the cross-validation model; and determining a prediction strength of the winning class for the eliminated sample based on the cross-validation model classification of the eliminated sample. The methods can further comprise filtering out any autoantibody binding activity values in the sample that exhibit an insignificant change, normalizing the autoantibody binding activity values of the vectors, and/or resealing the values. The method further comprises providing an output indicating the clusters (e.g., formed working clusters).

The invention also encompasses a method for ascertaining at least one previously unknown class (e.g., a cancer class) into which at least one sample to be tested is classified, wherein the sample is obtained from an individual. The method comprises obtaining autoantibody binding activity values for a plurality of epitopes from two or more samples; forming respective vectors of the samples, each vector being a series of autoantibody binding activity values indicative of autoantibody binding activities in a corresponding sample; and using a clustering routine, grouping vectors of the samples such that vectors indicative of similar autoantibody binding activities are clustered together (e.g., using a self organizing map) to form working clusters, the working clusters defining at least one previously unknown class. The previously unknown class is validated by using the methods for the weighted voting scheme described herein. The self organizing map is formed of a plurality of Nodes, N, and clusters the vectors according to a competitive learning routine. The competitive learning routine is:

fᵢ+1(N)=fᵢ(N)+τ(d(N,Nₚ),i)(P−fᵢ(N))

wherein i=number of iterations, N=the node of the self organizing map, τ=learning rate, P=the subject working vector, d=distance, Nₚ=node that is mapped nearest to P, and fᵢ(N) is the position of N at i.

The invention also provides a method for increasing the number of informative epitopes useful for a particular class prediction. The method involves determining the correlation of autoantibody binding activity for an epitope with a class distinction, and determining if the epitope is an informative epitope. In one embodiment, the method involves use of a signal to noise routine. If the epitope is determined to be informative, i.e. as having significant predictive value, it may be combined with other informative epitopes and used in accordance with a weighted voting scheme model as described herein for class prediction.

In one embodiment, the mean average antibody binding activity (SEM) for two or more epitopes across samples of a first class is compared to the mean average antibody binding activity (SEM) for the two or more epitopes across samples of a second class, and a neighborhood analysis using a two-sided Student t-test is done to identify informative epitopes.

In one embodiment, the invention provides a method for identifying a set of informative epitopes having autoantibody binding activities that correlate with a class distinction between samples, comprising the steps of: (a) determining autoantibody binding activities for a plurality of epitopes in a plurality of samples for each of two or more classes; (b) identifying clusters of epitopes from the plurality of epitopes which have autoantibody binding activities in samples of the same class from the plurality of samples, wherein the clusters of epitopes have autoantibody binding activities that correlate with a class distinction between samples of different classes from the plurality of samples; and (c) determining whether the correlation is stronger than expected by chance; wherein a cluster of epitopes having autoantibody binding activities that correlate with a class distinction more strongly than expected by chance are a set of informative epitopes.

In a preferred embodiment, a pattern recognition algorithm is used to identify a set of informative epitopes using autoantibody binding activities for a plurality of epitopes in a plurality of samples for each of two or more classes. The pattern recognition algorithm recognizes clusters of autoantibody binding activities that can be used to distinguish classes among the samples. In a preferred embodiment, the pattern recognition algorithm is used to validate the resulting patterns. In a preferred embodiment, a neural network pattern recognition algorithm is used. In another preferred embodiment, a support vector machine algorithm is used for pattern recognition. When a small number of samples are used, a support vector machine algorithm is preferably used. Training may be done using samples from any class that is to be distinguished, e.g., cancer samples or control samples.

The invention also pertains to a computer apparatus for classifying a sample into a class, wherein the sample is obtained from an individual, wherein the apparatus comprises: a source of autoantibody binding activity values of the sample; a processor routine executed by a digital processor, coupled to receive the autoantibody binding activity values from the source, the processor routine determining classification of the sample by comparing the autoantibody binding activity values of the sample to a model built with a weighted voting scheme or a pattern recognition algorithm and training samples; and an output assembly, coupled to the digital processor, for providing an indication of the classification of the sample. The model is built with a weighted voting scheme, as described herein, or a pattern recognition algorithm and training samples, as described herein. The output assembly comprises a display of the classification.

Yet another embodiment is a computer apparatus for constructing a model for classifying at least one sample to be tested, wherein the apparatus comprises a source of vectors for autoantibody binding activity values from two or more samples belonging to two or more classes, the vectors being a series of autoantibody binding activity values for the samples; a processor routine executed by a digital processor, coupled to receive the autoantibody binding activity values of the vectors from the source, the processor routine determining relevant epitopes for classifying the sample based on the autoantibody binding activity values, and constructing the model with a portion of the relevant epitopes by utilizing a weighted voting scheme. The apparatus can further include a filter, coupled between the source and the processor routine, for filtering out any of the autoantibody binding activity values in a sample that exhibit an insignificant change; or a normalizer, coupled to the filter, for normalizing the autoantibody binding activity values. The output assembly can be a graphical representation.

The invention also includes a computer apparatus for constructing a model for classifying at least one sample to be tested, wherein the model is based on autoantibody binding activity patterns established through the use of a pattern recognition algorithm and training samples.

The invention also involves a machine readable computer assembly for classifying a sample into a class, wherein the sample is obtained from an individual, wherein the computer assembly comprises a source of autoantibody binding activity values of the sample; a processor routine executed by a digital processor, coupled to receive the autoantibody binding activity values from the source, the processor routine determining classification of the sample by comparing the autoantibody binding activity values of the sample to a model built with a weighted voting scheme; and an output assembly, coupled to the digital processor, for providing an indication of the classification of the sample. The invention also includes a machine readable computer assembly for constructing a model for classifying at least one sample to be tested, wherein the computer assembly comprises a source of vectors for autoantibody binding activity values from two or more samples belonging to two or more classes, the vector being a series of autoantibody binding activity values for the samples; a processor routine executed by a digital processor, coupled to receive the autoantibody binding activity values of the vectors from the source, the processor routine determining relevant epitopes for classifying the sample, and constructing the model with a portion of the relevant epitopes by utilizing a weighted voting scheme.

The invention also includes a machine readable computer assembly for classifying a sample into a class, comprising a processor routine executed by a digital processor, wherein the processor routine determines classification of the sample by comparing autoantibody binding activities of the sample to a model based on autoantibody binding activity patterns established through the use of a pattern recognition algorithm and training samples.

In one embodiment, the invention includes a method of determining a treatment plan for an individual having a disease, comprising obtaining a sample from the individual; assessing autoantibody binding activity of the sample for at least one epitope; using a computer model built with a weighted voting scheme, classifying the sample into a disease class as a function of the autoantibody binding activity of the sample with respect to that of the model; and using the disease class, determining a treatment plan. Another application is a method of diagnosing or aiding in the diagnosis of an individual wherein a sample from the individual is obtained, comprising assessing the sample for autoantibody binding activity for at least one epitope; and using a computer model built with a weighted voting scheme, classifying the sample into a class of the disease including evaluating the autoantibody binding activity of the sample with respect to that of the model; and diagnosing or aiding in the diagnosis of the individual. The invention also includes a method for determining the efficacy of a drug designed to treat a disease class, wherein an individual has been subjected to the drug, which method comprises obtaining a sample from the individual subjected to the drug; assessing the sample for autoantibody binding activity for at least one epitope; and using a model built with a weighted voting scheme, classifying the sample into a class of the disease including evaluating the autoantibody binding activity of the sample as compared to that of the model. Yet another application is a method of determining whether an individual belongs to a phenotypic class that comprises obtaining a sample from the individual; assessing the sample for the autoantibody binding activity for at least one epitope; and using a model built with a weighted voting scheme, classifying the sample into a class including evaluating the autoantibody binding activity of the sample as compared to that of the model.

In another embodiment, the method of determining a treatment plan involves assessing the autoantibody binding activity of a patient sample for two or more epitopes using a computer model based on autoantibody binding activity patterns established through the use of a pattern recognition algorithm and training samples.

In one aspect, the invention provides a set of epitopes informative for breast cancer diagnosis. In a preferred embodiment, the invention provides a set of informative epitopes, which epitopes are informative for the diagnosis of breast cancer, comprising from 1-27, more preferably from 2-27, more preferably from 5-27, more preferably from 10-27, more preferably from 15-27, more preferably from 20-27, more preferably from 25-27 informative epitopes selected from the group consisting of those disclosed in FIG. 2. In a preferred embodiment, the set of informative epitopes comprises those disclosed in FIG. 2. In another preferred embodiment, the set of informative epitopes consists essentially of those disclosed in FIG. 2.

In another preferred embodiment, the invention provides a set of informative epitopes, which epitopes are informative for the diagnosis of lung cancer, particularly NSCLC, comprising from 1-51, more preferably from 2-51, more preferably from 5-51, more preferably from 10-51, more preferably from 15-51, more preferably from 20-51, more preferably from 25-51, more preferably from 30-51, more preferably from 35-51, more preferably from 40-51, more preferably from 45-51 informative epitopes selected from the group consisting of those disclosed in Table 2. In a preferred embodiment, the set of informative epitopes comprises those disclosed in Table 2. In another preferred embodiment, the set of informative epitopes consists essentially of those disclosed in Table 2.

In one aspect, the invention provides a set of epitopes informative for distinguishing NSCLC and SCLC. In a preferred embodiment, the invention provides a set of informative epitopes, which epitopes are informative for the distinguishing NSCLC and SCLC, comprising from 1-28, more preferably from 2-28, more preferably from 5-28, more preferably from 10-28, more preferably from 15-28, more preferably from 20-28, more preferably from 25-28 informative epitopes selected from the group consisting of those disclosed in FIG. 3. In a preferred embodiment, the set of informative epitopes comprises those disclosed in FIG. 3. In another preferred embodiment, the set of informative epitopes consists essentially of those disclosed in FIG. 3.

In one aspect, the invention provides a set of epitopes informative for distinguishing NSCLC and SCLC. In a preferred embodiment, the invention provides a set of informative epitopes, which epitopes are informative for the distinguishing NSCLC and SCLC, comprising from 1-51, more preferably from 2-51, more preferably from 5-51, more preferably from 10-51, more preferably from 15-51, more preferably from 20-51, more preferably from 25-51, more preferably from 30-51, more preferably from 35-51, more preferably from 40-51, more preferably from 45-51 informative epitopes selected from the group consisting of those disclosed in Table 2. In a preferred embodiment, the set of informative epitopes comprises those disclosed in Table 2. In another preferred embodiment, the set of informative epitopes consists essentially of those disclosed in Table 2.

In another preferred embodiment, the invention provides a set of informative epitopes, which epitopes are informative for the diagnosis of lung cancer, particularly NSCLC, comprising from 1-25, more preferably from 2-25, more preferably from 5-25, more preferably from 10-25, more preferably from 15-25, more preferably from 20-25 informative epitopes selected from the group consisting of those disclosed in Table 11. In a preferred embodiment, the set of informative epitopes comprises those disclosed in Table 11. In another preferred embodiment, the set of informative epitopes consists essentially of those disclosed in Table 11.

In one aspect, the invention provides sets of peptides useful for identifying a set of informative epitopes for a particular class distinction. In one embodiment, the set of peptides comprises from 1-1448, more preferably from 2-1448, more preferably from 5-1448, more preferably from 10-1448, more preferably from 25-1448, more preferably from 50-1448, more preferably from 100-1448, more preferably from 250-1448, more preferably from 500-1448, more preferably from 750-1448, more preferably from 1000-1448, more preferably from 1250-1448 peptides selected from the group of peptides disclosed in Table 1, and/or from 1-31, more preferably from 2-31, more preferably from 5-31, more preferably from 10-31, more preferably from 15-31, more preferably from 20-31, more preferably from 25-31 peptides selected from the group of peptides disclosed in Table 10, and/or from 1-83, more preferably 2-83, more preferably 5-83, more preferably 10-83, more preferably 15-83, more preferably 20-83, more preferably 25-83, more preferably 50-83, more preferably 75-83 peptides selected from the group of peptides disclosed in Table 9, and/or from 1-42, more preferably 2-42, more preferably 5-42, more preferably 10-42, more preferably 15-42, more preferably 20-42, more preferably 25-42, more preferably 30-42, more preferably 35-42 peptides selected from the group of peptides disclosed in Table 8, and/or from 1-52, more preferably from 2-52, more preferably from 5-52, more preferably from 10-52, more preferably from 15-52, more preferably from 20-52, more preferably from 25-52, more preferably from 30-52, more preferably from 35-52, more preferably from 40-52, more preferably from 45-52 peptides selected from the group of peptides disclosed in Table 7.

In one aspect, the invention provides epitope microarrays for distinguishing between a plurality of classes for a biological sample, wherein the microarray comprises a plurality of peptides, each peptide independently having a corresponding epitope binding activity in a sample characteristic of a particular class selected from the plurality of particular classes, wherein taken together, the plurality of peptides have corresponding epitope binding activities in a plurality of samples collectively characteristic of all of the plurality of particular classes, wherein the autoantibody binding activity of each peptide is independently higher in a sample characteristic of one of the plurality of particular classes than in a sample characteristic of another one of the plurality of particular classes.

In a preferred embodiment, the invention provides epitope microarrays for distinguishing between a first class and a second class for a biological sample. The epitope microarrays comprise a plurality of peptides, each peptide independently having a corresponding epitope binding activity in a sample characteristic of the first class or in a sample characteristic of the second class, wherein taken together, the plurality of peptides have corresponding epitope binding activities in samples collectively characteristic of the first and second classes, wherein the autoantibody binding activity of each peptide is independently higher in a sample characteristic of either the first class or the second class as compared to its autoantibody binding activity in a sample characteristic of the other class.

Preferred distinct classes include a non-disease class and a disease class, more preferably a non-cancer class and a cancer class, the latter preferably being lung cancer, breast cancer, gastrointestinal cancer, or prostate cancer. Other preferred distinct classes are a high risk class and a non-disease class, preferably a high risk cancer class and a non-cancer class. Other preferred distinct classes are distinct cancer classes, such as distinct lung cancer classes, such as NSCLC and SCLC. Other preferred distinct cancer classes are metastatic cancer and non-metastatic cancer classes.

In a preferred embodiment, two or more peptides of the epitope microarray correspond to distinct regions of a single protein, preferably non-overlapping regions of the single protein.

In another preferred embodiment, the invention provides an epitope microarray useful for the diagnosis of lung cancer, particularly NSCLC, which array comprises from 1-25, more preferably from 2-25, more preferably from 5-25, more preferably from 10-25, more preferably from 15-25, more preferably from 20-25 informative epitopes selected from the group consisting of those disclosed in Table 11. In a preferred embodiment, the set of informative epitopes comprises those disclosed in Table 11. In another preferred embodiment, the set of informative epitopes consists essentially of those disclosed in Table 11.

In another preferred embodiment, the invention provides an epitope microarray useful for the diagnosis of lung cancer, particularly NSCLC, which array comprises from 1-51, more preferably from 2-51, more preferably from 5-51, more preferably from 10-51, more preferably from 15-51, more preferably from 20-51, more preferably from 25-51, more preferably from 30-51, more preferably from 35-51, more preferably from 40-51, more preferably from 45-51 informative epitopes selected from the group consisting of those disclosed in Table 2. In a preferred embodiment, the set of informative epitopes comprises those disclosed in Table 2. In another preferred embodiment, the set of informative epitopes consists essentially of those disclosed in Table 2.

In another preferred embodiment, the invention provides an epitope microarray useful for the diagnosis of breast cancer, which array comprises from 1-27, more preferably from 2-27, more preferably from 5-27, more preferably from 10-27, more preferably from 15-27, more preferably from 20-27, more preferably from 25-27 informative epitopes selected from the group consisting of those disclosed in FIG. 2. In a preferred embodiment, the set of informative epitopes comprises those disclosed in FIG. 2. In another preferred embodiment, the set of informative epitopes consists essentially of those disclosed in FIG. 2.

In another preferred embodiment, the invention provides an epitope microarray useful for distinguishing between NSCLC and SCLC, which array comprises from 1-51, more preferably from 2-51, more preferably from 5-51, more preferably from 10-51, more preferably from 15-51, more preferably from 20-51, more preferably from 25-51, more preferably from 30-51, more preferably from 35-51, more preferably from 40-51, more preferably from 45-51 informative epitopes selected from the group consisting of those disclosed in Table 2. In a preferred embodiment, the set of informative epitopes comprises those disclosed in Table 2. In another preferred embodiment, the set of informative epitopes consists essentially of those disclosed in Table 2.

In another preferred embodiment, the invention provides an epitope microarray useful for distinguishing between NSCLC and SCLC, which array comprises from 1-28, more preferably from 2-28, more preferably from 5-28, more preferably from 10-28, more preferably from 15-28, more preferably from 20-28, more preferably from 25-28 informative epitopes selected from the group consisting of those disclosed in FIG. 3. In a preferred embodiment, the set of informative epitopes comprises those disclosed in FIG. 3. In another preferred embodiment, the set of informative epitopes consists essentially of those disclosed in FIG. 3.

In a preferred embodiment, the invention provides an epitope microarray useful for identifying informative epitopes for a particular class distinction. The epitope microarray comprises from 1-1448, more preferably from 2-1448, more preferably from 5-1448, more preferably from 10-1448, more preferably from 25-1448, more preferably from 50-1448, more preferably from 100-1448, more preferably from 250-1448, more preferably from 500-1448, more preferably from 750-1448, more preferably from 1000-1448, more preferably from 1250-1448 peptides selected from the group of peptides disclosed in Table 1, and/or from 1-31, more preferably from 2-31, more preferably from 5-31, more preferably from 10-31, more preferably from 15-31, more preferably from 20-31, more preferably from 25-31 peptides selected from the group of peptides disclosed in Table 10, and/or from 1-83, more preferably 2-83, more preferably 5-83, more preferably 10-83, more preferably 15-83, more preferably 20-83, more preferably 25-83, more preferably 50-83, more preferably 75-83 peptides selected from the group of peptides disclosed in Table 9, and/or from 1-42, more preferably 2-42, more preferably 5-42, more preferably 10-42, more preferably 15-42, more preferably 20-42, more preferably 25-42, more preferably 30-42, more preferably 35-42 peptides selected from the group of peptides disclosed in Table 8, and/or from 1-52, more preferably from 2-52, more preferably from 5-52, more preferably from 10-52, more preferably from 15-52, more preferably from 20-52, more preferably from 25-52, more preferably from 30-52, more preferably from 35-52, more preferably from 40-52, more preferably from 45-52 peptides selected from the group of peptides disclosed in Table 7.

In one embodiment, the invention provides an epitope microarray useful for distinguishing between two or more classes and, accordingly, for predicting the classification of a sample, comprising a set of informative epitopes for class distinction that are selected using the methods disclosed herein.

### BRIEF DESCRIPTION OF THE DRAWINGS

FIG. 1. Epitope microarray design. Both arrays were hybridized with the same serum and the peptide-aAb complexes detected by a secondary anti-Human Ig conjugated to either (A) alkaline phosphatase or (B) Cy3. Similar signal patterns were obtained using these two independent detection methods. Thus, the epitope microarray is compatible with different detection methods. (C) The IgG serial dilutions for data normalization. PC—positive control; NC—negative control.

FIG. 2. Sample set of breast cancer informative epitopes. A set of informative epitopes for breast cancer was determined using two-sided t-test assuming equal variance, and then sorted into two groups based on I/D signal dichotomy. EB and EC were determined as described in the experimental section.

FIG. 3. Sample set of lung cancer informative epitopes. A set of lung cancer informative epitopes was determined using Student t-test, and then sorted into two groups based on I/D signal dichotomy. EN and ES were determined as described in the experimental section.

FIG. 4. Clustering of our results compared with previously published cancer survival data (see Marcus et al., J Natl Cancer Inst. 92:1308-16 (2000).

FIG. 5. Epitope evaluation and signal analysis. Signal strength in each patient and control individual is expressed on a scale of five. A pair-wise epitope signal comparison is then carried out for each individual epitope. Only the epitopes producing a significantly different signal (p<0.05) are then used to compose the marker sets that differentiate between two groups. All epitopes in this figure are considered informative for breast cancer because they all produced a signal that was significantly different in breast cancer compared with non-cancer control.

### DETAILED DESCRIPTION

“Autoantibody binding activity” and “autoantibody binding activity value” refers to the measure of the binding interaction between a given epitope and an autoantibody in a given sample, which is a semiquantifiable measure that is reflective of the amount of epitope-binding autoantibody in the sample. As used herein, the autoantibody binding activity “of a sample”, “in a sample”, “with a sample”, or “for a sample”, refers to the measure of the binding interaction between a given epitope and an autoantibody in the given sample.

“Epitope binding activity” as used herein refers to an epitope-binding autoantibody in a sample. A “corresponding epitope binding activity” for a particular epitope is an autoantibody that specifically binds the particular epitope.

“Autoantibodies” (“aABs”) specifically bind components of the same body that produces them. Altered serum autoantibody composition has been noted in a number of different cancers including breast (Metcalfe et al., Breast Cancer Res. 2:438-43 (2000)) and lung cancer (Lubin et al., Nat Med. 1:701-2 (1995); Blaes et al., Ann Thorac Surg. 69:254-8 (2000); Gure et al., Cancer Res. 58:1034-41 (1998)), and a variety of other diseases including lupus erythematosus, Sjogren's syndrome, scleroderma, dermato/polymyositis, type I diabetes, paraneoplastic neuronal syndromes, inflammatory bowel disease and thyroid endocrinopathies (see Schwarz, Autoimmunity and Autoimmune Disease, In: Fundamental Immunology, 3rd ed. (Ed. Paul WE) pp. 1033-99 Raven Press, New York, 1993).

The methods disclosed herein generally relate to two areas: class prediction and class discovery. Class prediction refers to the assignment of particular samples to defined classes which may reflect current states, predispositions, or future outcomes. Class discovery refers to defining one or more previously unrecognized biological classes.

In one aspect, the invention relates to predicting or determining a classification of a sample, comprising identifying a set of informative epitopes whose autoantibody binding activities correlate with a class distinction among samples. In one embodiment, the method involves sorting epitopes by the degree to which autoantibody binding thereto across all the samples correlates with the class distinction, and then determining whether the correlation is stronger than expected by chance (i.e., statistically significant). If the correlation of autoantibody binding activity with class distinction is statistically significant, that epitope is considered an “informative” or “relevant” epitope.

Related classification methods based on gene expression profiling have been described previously. See Golub et al., U.S. Pat. No. 6,647,341, expressly incorporated herein in its entirety by reference. Notably, the present invention differs from the disclosure of Golub et al. in that the present classification schemes and methods do not involve measurements of gene expression. Rather, the present methods involve measurements of immune status based on the binding of autoantibodies in biological samples to peptide epitopes. The present invention stems from the finding that the immune status evidenced by a sample's autoantibody binding activities is highly informative in respect of biological class distinctions, given an appropriate set of informative epitopes.

Once a set of informative epitopes is identified, the weight given the information provided by each informative epitope is determined. Each vote is a measure of how much the new sample's level of autoantibody binding activity looks like the typical level of autoantibody binding activity in training samples from a particular class. The more strongly autoantibody binding activity is correlated with a class distinction, the greater the weight given to the information which that epitope provides. In other words, if autoantibody binding to a particular epitope is strongly correlated with a class distinction, that epitope will carry a great deal of weight in determining the class to which a sample belongs. Conversely, if autoantibody binding to a particular epitope is only weakly correlated with a class distinction, that epitope will be given little weight in determining the class to which a sample belongs. Each informative epitope to be used from the set of informative epitopes is assigned a weight. It is not necessary that the complete set of informative epitopes be used; a subset of the total informative epitopes can be used as desired. Using this process, a weighted voting scheme may be determined, and a predictor or model for class distinction may be created from a set of informative epitopes.

A further aspect of the invention includes assigning a biological sample to a known or putative class (i.e., class prediction) by evaluating the sample's autoantibody binding activity for informative epitopes. For each informative epitope, a vote for one or the other class is determined based on autoantibody binding activity of the sample. Each vote is then weighted in accordance with the weighted voting scheme described above, and the weighted votes are summed to determined the winning class for the sample. The winning class is defined as the class for which the largest vote is cast. Optionally, a prediction strength (PS) for the winning class can also be determined. Prediction strength is the margin of victory of the winning class that ranges from 0 to 1. In one embodiment, a sample can be assigned to the winning class only if the PS exceeds a certain threshold (e.g., 0.3); otherwise the assessment is considered uncertain.

In another embodiment, a pattern recognition algorithm is used with training samples characteristic of a particular class. The particular class of samples used may be any one of those that are to be distinguished between. For example, samples characteristic of a cancer class, or samples characteristic of a non-cancer class may be used with a pattern recognition algorithm to generate a model useful for distinguishing between cancer and non-cancer samples.

In one embodiment, a support vector machine algorithm is used. In another embodiment, a neural network algorithm is used. Preferably, if a small number of training samples are used, a support vector machine algorithm is used.

Another embodiment of the invention relates to a method of discovering or ascertaining two or more classes from samples by clustering the samples based on autoantibody binding activities to obtain putative classes (i.e., class discovery). The putative classes are validated by carrying out the class prediction steps, as described above. In preferred embodiments, one or more steps of the methods are performed using a suitable processing means, e.g., a computer.

In one embodiment, the methods of the present invention are used to classify a sample with respect to a specific disease class or a subclass within a specific disease class. The invention is useful in classifying a sample for virtually any disease, condition, or syndrome including, but not limited to, cancer, autoimmune diseases, infectious diseases, neurodegenerative diseases, etc. That is, the invention can be used to determine whether a sample belongs to (is classified as) a specific disease category (e.g., extant lung cancer, as opposed to non-cancer, as opposed to high risk for manifestation of lung cancer) and/or to a class within a specific disease (e.g., small cell lung cancer (“SCLC”) class as opposed to non-small cell lung cancer (“NSCLC”) class).

As used herein, the terms “class” and “subclass” are intended to mean a group which shares one or more characteristics. For example, a disease class can be broad (e.g., proliferative disorders), intermediate (e.g., cancer) or narrow (e.g., lung cancer). The term “subclass” is intended to further define or differentiate a class. For example, in the class of lung cancer, NSCLC and SCLC are examples of subclasses; however, NSCLC and SCLC can also be considered as classes in and of themselves. These terms are not intended to impart any particular limitations in terms of the number of group members. Rather, they are intended only to assist in organizing the different sets and subsets of groups as biological distinctions are made.

The invention can be used to identify classes or subclasses between samples with respect to virtually any category or response, and can be used to classify a given sample with respect to that category or response. In one embodiment the class or subclass is previously known. For example, the invention can be used to classify samples, based on autoantibody binding activities, as being from individuals who are more susceptible to viral (e.g., HIV, human papilloma virus, meningitis) or bacterial (e.g., chlamydial, staphylococcal, streptococcal) infection versus individuals who are less susceptible to such infections. The invention can be used to classify samples based on any phenotypic or physiological trait, including, but not limited to, cancer, obesity, diabetes, high blood pressure, response to chemotherapy, etc. The invention can further be used to identify previously unknown biological classes.

In particular embodiments, class prediction is carried out using samples from individuals known to have the disease type or class being studied, as well as samples from individuals not having the disease or having a different type or class of the disease. This provides the ability to assess autoantibody binding activity patterns across the full range of phenotypes. Using the methods described herein, a classification model is built with the autoantibody binding activities from these samples.

In one embodiment, this model is created by identifying a set of informative or relevant epitopes, for which the autoantibody binding activity in samples is correlated with the class distinction to be predicted. For example, the epitopes are sorted by the degree to which their autoantibody binding activities correlate with the class distinction, and this data is assessed to determine whether the observed correlations are stronger than would be expected by chance (e.g., are statistically significant). If the correlation for a particular epitope is statistically significant, then the epitope is considered an informative epitope. If the correlation is not statistically significant, then the epitope is not considered an informative epitope.

The degree of correlation between autoantibody binding activity and class distinction can be assessed using a number of methods. In a preferred embodiment, each epitope is represented by an autoantibody binding activity vector v(g)=(a₁, a₂, . . . , an), where al denotes the autoantibody binding activity of epitope g in i^{th} sample in the initial set (S) of samples. A class distinction is represented by an idealized autoantibody binding activity pattern c=(c₁, c₂, . . . , cₙ), where cᵢ=+1 or 0 according to whether the i^{th} sample belongs to class 1 or class 2. The correlation between an epitope and a class distinction can be measured in a variety of ways. Suitable methods include, for example, the Pearson correlation coefficient r(g,c) or the Euclidean distance d(g*,c*) between normalized vectors (where the vectors g* and c* have been normalized to have mean 0 and standard deviation 1).

In a preferred embodiment, the correlation is assessed using a measure of correlation that emphasizes the “signal-to-noise” ratio in using the epitope as a predictor. In this embodiment, (μ₁ (g), σ₁(g)) and (μ₂(g),σ₂(g)) denote the means and standard deviations of the log₁₀ of the autoantibody binding values of epitope g for the samples in class 1 and class 2, respectively. P(g,c)=(μ₁(g)−μ₂(g))/(σ₁(g)+σ₂(g)), which reflects the difference between the classes relative to the standard deviation within the classes. Large values of |P(g,c)| indicate a strong correlation between the autoantibody binding activity and the class distinction, while small values of |P(g,c)| indicate a weak correlation between autoantibody binding activity and class distinction. The sign of P(g,c) being positive or negative corresponds to g having greater autoantibody binding activity in class 1 or class 2, respectively. Note that P(g,c), unlike a standard Pearson correlation coefficient, is not confined to the range [−1,+1]. If N₁(c,r) denotes the set of genes such that P(g,c)>=r, and if N₂(c,r) denotes the set of epitopes such that P(g,c)<=r, N₁(c,r) and N₂(c,r) are the neighborhoods of radius r around class 1 and class 2. An unusually large number of epitopes within the neighborhoods indicates that many epitopes have autoantibody binding activity patterns closely correlated with the class vector.

An assessment of whether the observed correlations are stronger than would be expected by chance is most preferably carried out using a “neighborhood analysis”. In this method, an idealized pattern corresponding to autoantibody binding activity that is uniformly high in one class and uniformly low in the other class is defined, and one tests whether there is an unusually high density of autoantibody binding activities “nearby” or “in the neighborhood of”, i.e., more similar to, the idealized pattern than equivalent random patterns. The determination of whether the density of nearby autoantibody binding activities is statistically significantly higher than expected can be carried out using known methods for determining the statistical significance of differences. One preferred method is a permutation test in which the number of autoantibody binding activities in the neighborhood (nearby) is compared to the number of autoantibody binding activities in similar neighborhoods around idealized patterns corresponding to random class distinctions, obtained by permuting the coordinates of c.

The sample assessed can be any sample that can contain epitope-binding autoantibodies. Preferred samples are serum samples from individuals. Also preferred are samples of synovial fluid and cerebrospinal fluid. Using the methods described herein, the autoantibody binding activities for a plurality of epitopes can be measured simultaneously. The assessment of numerous autoantibody binding activities (autoantibody profiling) provides for a more accurate evaluation of the sample because there are more autoantibody binding activities that can assist in classifying the sample.

The autoantibody binding activities are obtained, e.g., by contacting the sample with a suitable epitope microarray, and determining the extent of binding of autoantibodies in the sample to the epitopes on the microarray. Once the autoantibody binding activities of the sample are obtained, they are compared or evaluated against the model, and then the sample is classified. The evaluation of the sample determines whether or not the sample should be assigned to the particular class being studied.

The autoantibody binding activity measured or assessed is the numeric value obtained from an apparatus that can measure autoantibody binding activity levels. Autoantibody binding activity values refer to the amount of autoantibody binding detected for a given epitope, as described herein. The values are raw values from the apparatus, or values that are optionally, rescaled, filtered and/or normalized. Such data is obtained, for example, from an epitope microarray platform using fluorometry-based or colorimetric autoantibody detection techniques.

The data can optionally be prepared by using a combination of the following: rescaling data, filtering data and normalizing data. The autoantibody binding activity values can be rescaled to account for variables across experiments or conditions, or to adjust for minor differences in overall array intensity. Such variables depend on the experimental design the researcher chooses. The preparation of the data sometimes also involves filtering and/or normalizing the values prior to subjecting the autoantibody binding activity values to clustering.

Filtering the autoantibody binding activity values involves eliminating any vector in which the autoantibody binding activity value exhibits no change or an insignificant change across samples. Once the autoantibody binding activities for epitopes are filtered then the subset of epitopes/autoantibody binding activities that remain are referred to herein “working vectors.”

The present invention can also involve normalizing the levels of autoantibody binding activity values. The normalization of autoantibody binding activity values is not always necessary and depends on the type or algorithm used to determine the correlation between autoantibody binding activity and a class distinction. The absolute level of autoantibody binding activity is not as important as the degree of correlation autoantibody binding activity has for a particular class. Normalization occurs using the following equation:

NV=(ABV−AABV)/SDV

wherein NV is the normalized value, ABV is the autoantibody binding activity value across samples, AABV is the average autoantibody binding activity value across samples, and SDV is the standard deviation of the autoantibody binding activity values.

Once the autoantibody binding activity values are prepared, then the data is classified or is used to build the model for classification. Epitopes that are relevant for classification are first determined. The term “relevant epitopes” refers to those epitopes for which autoantibody binding activity correlates with a class distinction. The epitopes that are relevant for classification are also referred to herein as “informative epitopes”. The correlation between autoantibody binding activity and class distinction can be determined using a variety of methods; for example, a neighborhood analysis can be used. A neighborhood analysis comprises performing a permutation test, and determining probability of number of genes in the neighborhood of the class distinction, as compared to the neighborhoods of random class distinctions. The size or radius of the neighborhood is determined using a distance metric. For example, the neighborhood analysis can employ the Pearson correlation coefficient, the Euclidean distance coefficient, or a signal to noise coefficient. The relevant epitopes are determined by employing, for example, a neighborhood analysis which defines an idealized autoantibody binding activity pattern corresponding to a autoantibody binding activity that is uniformly high in one class and uniformly low in other class(es). A disparity in autoantibody binding activity exists when comparing the level of autoantibody binding activity in one class with other classes. Such epitopes are good indicators for evaluating and classifying a sample based on its autoantibody binding activities. In one embodiment, the neighborhood analysis utilizes the following signal to noise routine:

P(g,c)=(μ₁(g)−μ₂(g))/(σ₁(g)+σ₂(g)),

wherein g is the autoantibody binding activity value for a given epitope; c is the class distinction, μ₁(g) is the mean of the autoantibody binding activities for g for a first class; μ₂(g) is the mean of the autoantibody binding activities for g for a second class; σ₁(g) is the standard deviation for g the first class; and σ₂(g) is the standard deviation for the second class. The invention includes classifying a sample into one of two classes, or into one of multiple (a plurality of) classes.

Particularly relevant epitopes are those that are best suited for classifying samples. The step of determining the relevant epitopes also provides means for isolating antibodies that can be used to identify immunogenic proteins potentially involved in manifestation of the class, e.g., proteins involved in pathogenesis. Consequently, the methods of the present invention also pertain to determining drug target(s) based on immunogenic proteins that specifically bind to epitope binding autoantibodies and are involved with the class (e.g., disease) being studied, and the drug, itself, as determined by this method.

The next step for classifying epitopes involves building or constructing a model or predictor that can be used to classify samples to be tested. One builds the model using samples for which the classification has already been ascertained, referred to herein as an “initial dataset.” Once the model is built, then a sample to be tested is evaluated against the model (e.g., classified as a function of the relative autoantibody binding activities of the sample with respect to that of the model).

A portion of the relevant epitopes, determined as described above, can be chosen to build the model. Not all of the epitopes need to be used. The number of relevant epitopes to be used for building the model can be determined by one of skill in the art. For example, out of 1000 epitopes that demonstrate a high correlation of autoantibody binding activity to a class distinction, 25, 50, 75 or 100 or more of these epitopes can be used to build the model.

The model or predictor is built using a “weighted voting scheme” or “weighted voting routine.” A weighted voting scheme allows these informative epitopes to cast weighted votes for one of the classes. The magnitude of the vote is dependant on both the autoantibody binding activity level and the degree of correlation of the autoantibody binding activity with the class distinction. The larger the disparity or difference between autoantibody binding activity from one class and the next, the larger the vote the epitope will cast. An epitope with a larger difference is a better indicator for class distinction, and so casts a larger vote.

The model is built according to the following weighted voting routine:

V_{g}=a_{g}(x_{g}−b_{g}),

wherein V_{g} is the weighted vote of the epitope, g; a_{g} is the correlation between autoantibody binding activity values for the epitope and class distinction, P(g,c), as defined herein; b_{g}=(μ₁(g)+μ₂ (g))/2 which is the average of the mean log₁₀ autoantibody binding activity value in a first class and a second class; x_{g} is the log₁₀ autoantibody binding activity value in the sample to be tested. A positive weighted vote is a vote for the new sample's membership in the first class, and a negative weighted vote is a vote for the new sample's membership in the second class. The total vote V₁ for the first class is obtained by summing the absolute values of the positive votes over the informative epitopes, while the total vote V₂ for the second class is obtained by summing the absolute values of the negative votes.

A prediction strength can also be measured to determine the degree of confidence the model classifies a sample to be tested. The prediction strength conveys the degree of confidence of the classification of the sample and evaluates when a sample cannot be classified. There may be instances in which a sample is tested, but does not belong to a particular class. This is done by utilizing a threshold wherein a sample which scores below the determined threshold is not a sample that can be classified (e.g., a “no call”). For example, if a model is built to determine whether a sample belongs to one of two lung cancer classes, but the sample is taken from an individual who does not have lung cancer, then the sample will be a “no call” and will not be able to be classified. The prediction strength threshold can be determined by the skilled artisan based on known factors, including, but not limited to the value of a false positive classification versus a “no call”.

Once the model is built, the validity of the model can be tested using methods known in the art. One way to test the validity of the model is by cross-validation of the dataset. To perform cross-validation, one of the samples is eliminated and the model is built, as described above, without the eliminated sample, forming a “cross-validation model.” The eliminated sample is then classified according to the model, as described herein. This process is done with all the samples of the initial dataset and an error rate is determined. The accuracy the model is then assessed. This model should classify samples to be tested with high accuracy for classes that are known, or classes have been previously ascertained or established through class discovery. Another way to validate the model is to apply the model to an independent data set. Other standard biological or medical research techniques, known or developed in the future, can be used to validate class discovery or class prediction.

The invention also provides a method for increasing the number of informative epitopes useful for a particular class prediction. The method involves determining the correlation of autoantibody binding activity for an epitope with a class distinction, and determining if the epitope is an informative epitope. In one embodiment, the method involves use of a signal to noise routine. If the epitope is determined to be informative, i.e. as having significant predictive value, it may be combined with other informative epitopes and used in accordance with a weighted voting scheme model as described herein for class prediction.

The invention also provides alternative means for determining whether epitopes are informative for a particular biological class distinction. For example, in one embodiment, the mean average antibody binding activity (±SEM) for two or more epitopes across samples of a first class is compared to the mean average antibody binding activity (±SEM) for the two or more epitopes across samples of a second class, and a two-sided Student t-test is done to identify informative epitopes.

An aspect of the invention also includes ascertaining or discovering classes that were not previously known, or validating previously hypothesized classes. This process is referred to herein as “class discovery.” This embodiment of the invention involves determining the class or classes not previously known, and then validating the class determination (e.g., verifying that the class determination is accurate).

To ascertain classes that were not previously known or recognized, or to validate classes which have been proposed on the basis of other findings, the samples are grouped or clustered based on autoantibody binding activities. The autoantibody binding activity pattern (i.e., aAB profile) of a sample and the samples having similar autoantibody binding activity patterns are grouped or clustered together. The group or cluster of samples identifies a class. This clustering methodology can be applied to identify any classes in which the classes differ based on their autoantibody binding activity patterns.

Determining classes that were not previously known is performed by the present methods using a clustering routine. The present invention can utilize several clustering routines to ascertain previously unknown classes, such as Bayesian clustering, k-means clustering, hierarchical clustering, and Self Organizing Map (SOM) clustering.

Once the autoantibody binding activity values are prepared, the data is clustered or grouped. One particular aspect of the invention utilizes SOMs, a competitive learning routine, for clustering autoantibody binding activity patterns to ascertain the classes. SOMs impose structure on the data, with neighboring nodes tending to define ‘related’ clusters or classes.

SOMs are constructed by first choosing a geometry of “nodes”. Preferably, a 2 dimensional grid (e.g., a 3×2 grid) is used, but other geometries can be used. The nodes are mapped into k-dimensional space, initially at random and then interactively adjusted. Each iteration involves randomly selecting a vector and moving the nodes in the direction of that vector. The closest node is moved the most, while other nodes are moved by smaller amounts depending on their distance from the closest node in the initial geometry. In this fashion, neighboring points in the initial geometry tend to be mapped to nearby points in k-dimensional space. The process continues for several (e.g., 20,000-50,000) iterations.

The number of nodes in the SOM can vary according to the data. For example, the user can increase the number of Nodes to obtain more clusters. The proper number of clusters allows for a better and more distinct representation of the particular cluster of samples. The grid size corresponds to the number of nodes. For example a 3×2 grid contains 6 nodes and a 4×5 grid contains 20 nodes. As the SOM algorithm is applied to the samples based on autoantibody binding activity data, the nodes move toward the sample cluster over several iterations. The number of Nodes directly relates to the number of clusters. Therefore, an increase in the number of Nodes results in an increase in the number of clusters. Having too few nodes tends to produce patterns that are not distinct. Additional clusters result in distinct, tight clusters of autoantibody binding activity. The addition of even more clusters beyond this point does not result any fundamentally new patterns. For example, one can choose a 3×2 grid, a 4×5 grid, and/or a 6×7 grid, and study the output to determine the most suitable grid size.

A variety of SOM algorithms exist that can cluster samples according to autoantibody binding activity vectors. The invention utilizes any SOM routine (e.g., a competitive learning routine that clusters the autoantibody binding activity patterns), and preferably, uses the following SOM routine:

fᵢ₊₁(N)=fᵢ(N)+τ(d(N,Nₚ),i)(P−fᵢ(N)),

wherein i=number of iterations, N=the node of the self organizing map, τ=learning rate, P=the subject working vector, d=distance, Nₚ=node that is mapped nearest to P, and fᵢ(N) is the position of N at i.

Once the samples are grouped into classes using a clustering routine, the putative classes are validated. The steps for classifying samples (e.g., class prediction) can be used to verify the classes. A model based on a weighted voting scheme, as described herein, is built using the autoantibody binding activity data from the same samples for which the class discovery was performed. Such a model will perform well (e.g., via cross validation and via classifying independent samples) when the classes have been properly determined or ascertained. If the newly discovered classes have not been properly determined, then the model will not perform well (e.g., not better than predicting by the majority class). All pairs of classes discovered by the chosen class discovery method may be compared. For each pair C₁, C₂, S is the set of samples in either C₁ or C₂. Class membership (either C₁ or C₂) is predicted for each sample in S by the cross validation method described herein. The median PS (over the |S| predictions) to be a measure of how predictable the class distinction is from the given data. A low median PS value (e.g., near 0.3) indicates either spurious class distinction or an insufficient amount of data to support a real distinction. A high median PS value (e.g., 0.8) indicates a strong, predictable class distinction.

The class discovery techniques above can be used to identify the fundamental subtypes of any disorder, e.g., cancer. Class discovery methods could also be used to search for fundamental immune mechanisms that cut across distinct types of cancers. For example, one might combine different cancers (for example, breast tumors and prostate tumors) into a single dataset and cluster the samples based on epitope binding activities. Moreover, in a preferred embodiment, the class predictor described herein is adapted to a clinical setting, with an appropriate epitope microarray as described herein.

Classification of the sample gives a healthcare provider information about a classification to which the sample belongs, based on the analysis or evaluation of autoantibody binding activity for multiple epitopes. The methods provide a more accurate assessment than traditional tests because multiple autoantibody binding activities or markers are analyzed, as opposed to analyzing one or two markers as is done for traditional tests. The information provided by the present invention, alone or in conjunction with other test results, aids the healthcare provider in diagnosing the individual.

Also, the present invention provides methods for determining a treatment plan. Once the health care provider knows to which disease class the sample, and therefore, the individual belongs, the health care provider can determine an adequate treatment plan for the individual. Different disease classes often require differing treatments. Properly diagnosing and understanding the class of disease of an individual allows for a better, more successful treatment and prognosis.

Other applications of the invention include ascertaining classes for or classifying persons who are likely to have successful treatment with a particular drug or regimen. Those interested in determining the efficacy of a drug can utilize the methods of the present invention. During a study of the drug or treatment being tested, individuals who have a disease may respond well to the drug or treatment, and others may not. Samples are obtained from individuals who have been subjected to the drug being tested and who have a predetermined response to the treatment. A model can be built from a portion of the relevant epitopes, using the weighted voting scheme described herein. A sample to be tested can then be evaluated against the model and classified on the basis of whether treatment would be successful or unsuccessful. The company testing the drug could provide more accurate information regarding the class of individuals for which the drug is most useful. This information also aids a healthcare provider in determining the best treatment plan for the individual.

Another application of the present invention is classification of a sample from an individual to determine the likelihood that a particular disease or condition will manifest in an individual. For example, persons who are more likely to contract heart disease or high blood pressure can have autoantibody binding activity profiles different from those who are less likely to suffer from these diseases. A model, using the methods described herein, can be built from individuals who have heart disease or high blood pressure, and those who do not using a weighted voting scheme. Once the model is built, a sample from an individual can be tested and evaluated with respect to the model to determine to which class the sample belongs. An individual who belongs to the class of individuals who have the disease, can take preventive measures (e.g., exercise, aspirin, etc.). Heart disease and high blood pressure are examples of diseases that can be classified, but the present invention can be used to classify samples for virtually any disease, including predispositions for cancer.

A preferred embodiment for identifying and predicting predisposition to disease involves building a weighted voting scheme model using the methods described herein with samples from individuals who do not have, but are at high risk for, a particular disease condition. An example of such an individual would be a long term high frequency smoker who has not presented with lung cancer, or a family member whose pedigree predicts occurrence of a familial disease, but who has not presented with the disease. Once the model is built, a sample from an individual can be tested and evaluated with respect to the model to determine to which class the sample belongs. An individual who belongs to the class of individuals predisposed to the disease can take preventive measures (e.g., exercise, aspirin, cessation of smoking, etc.).

More generally, class predictors may be useful in a variety of settings. First, class predictors can be constructed for known pathological categories, reflecting a tumor's cell of origin, stage or grade. Such predictors could provide diagnostic confirmation or clarify unusual cases. Second, the technique of class prediction can be applied to distinctions relating to future clinical outcome, such as drug response or survival.

### Epitope Microarrays

In one aspect, the invention provides epitope microarrays which are positionally addressable arrays of autoantibody-binding peptides (epitopes) adhered to the array. The array contains from two to thousands of epitopes, more preferably from 10-1,500, more preferably from 20-1000, more preferably from 50-500 epitopes. The epitopes used are preferably from about 3 to about 20, more preferably about 15 amino acids in length, though epitopes of other lengths may be used. A binding agent, preferably a secondary antibody that specifically binds to an autoantibody present in the sample, is used to detect the presence of the autoantibody specifically bound to an epitope of the array. The detection agent is preferably labeled with a detectable label, (e.g., ³²P, calorimetric indicator, or a fluorescent label), prior to incubation with the epitope array.

The choice of epitopes used for autoantibody detection, and for epitope microarrays, may depend on the class distinction desired. Alternatively, a set of random peptides may be used and informative epitopes within the set may be identified using the methods disclosed herein.

In a preferred embodiment, the invention provides epitope microarrays useful for the diagnosis of cancer, and peptides present on such microarrays are selected from a set designed based on the following scheme. A first group of epitopes of the set corresponds to proteins that are expressed in embryonal tissues, and whose aberrant expression in adult tissues could provoke a humoral immune response. These include transcription factors (TFs) that are active in embryonal development, and also elicit immune responses while expressed in tumor cells. For example, aAbs against the members of SOX-family transcription factors have been identified in the sera of small cell lung cancer (SCLC) patients (Gure et al. supra). The members of SOX-family TFs are normally expressed in the developing nervous system and their expression has not been documented in normal lung epithelium (Gure et al. supra). Furthermore, expression of the members of basic helix-loop-helix (bHLH) family TFs that play a role in embryonal nervous system has been documented in NSCLC and SCLC (Chen et al., Proc Natl Acad Sci USA. (1997) 94:5355-60).

Additionally, the cancer diagnostic epitope microarray preferably incorporates previously published B-cell epitopes and the epitopes predicted to bind various isoforms of class 11 major histocompatibility complex (MHC). Publicly available MHC II binding algorithms such as ProPred and RankPept may be used. Special attention in epitope design is given to proteins whose autoantibodies have been linked to cancer. These include p53 and various members of SOX, FOX, IMP, ELAV/HU and other families (Tan, J Clin Invest. (2001) 108:1411-5). Also preferably included on the cancer diagnostic microarray are epitopes known to trigger a T-cell response, as an overlap between the T- and B-immunogenicity could be inferred from previous studies (Scanlan et al., Cancer Immun. (2001) 1:4; Chen et al., Proc Natl Acad Sci USA. (1998) 95:6919-23). An excellent collection of known T-cell epitopes exist in Cancer Immunity database. Thus, a highly preferred cancer diagnostic epitope microarray combines previously identified immunogenic sequences with the embryonal factor epitope design described above. The peptides are synthesized and may be printed on a microarray using known methods. For example, see Robinson et al., supra.

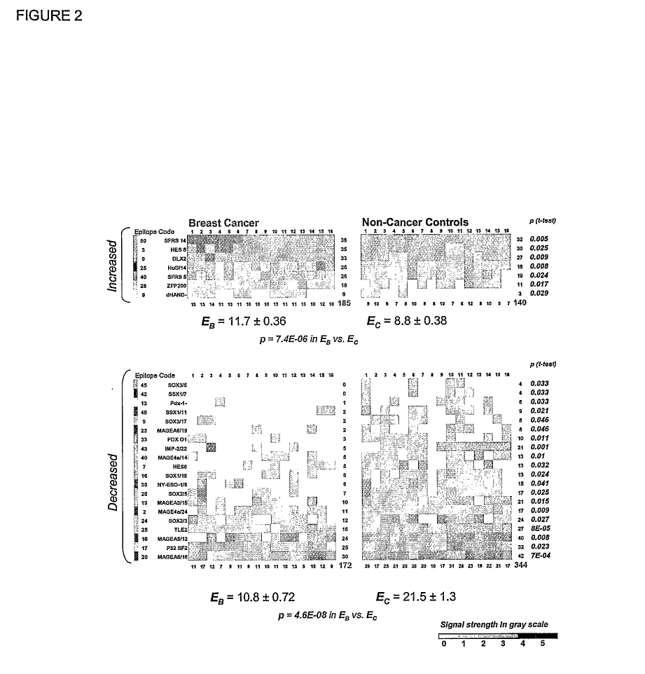

Preferred informative epitopes for the diagnosis of breast cancer include those disclosed in FIG. 2.

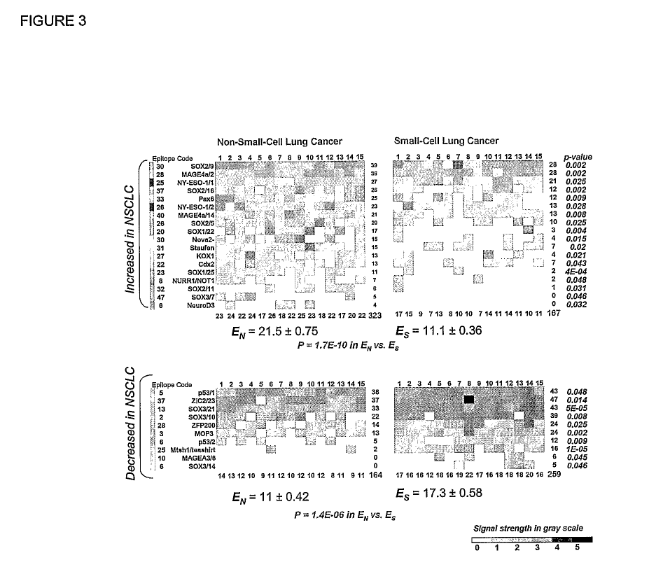

Preferred informative epitopes for distinguishing between NSCLC and SCLC include those disclosed in FIGS. 3, 7, and 13.

Preferred informative epitopes for the diagnosis of NSCLC include those disclosed in FIGS. 7 and 13.

Preferred epitopes from which to select informative epitopes for predicting a class distinction include those disclosed in FIGS. 6, 7, 9, 10, 11, 12, and 13.

In one aspect, the invention provides epitope microarrays for distinguishing between a plurality of classes for a biological sample, wherein the microarray comprises a plurality of peptides, each peptide independently having a corresponding epitope binding activity in a sample characteristic of a particular class selected from the plurality of particular classes, wherein taken together, the plurality of peptides have corresponding epitope binding activities in a plurality of samples collectively characteristic of all of the plurality of particular classes, wherein the autoantibody binding activity of each peptide is independently higher in a sample characteristic of one of the plurality of particular classes than in a sample characteristic of another one of the plurality of particular classes.

In a preferred embodiment, the invention provides epitope microarrays for distinguishing between a first class and a second class for a biological sample. The epitope microarrays comprise a plurality of peptides, each peptide independently having a corresponding epitope binding activity in a sample characteristic of the first class or in a sample characteristic of the second class, wherein taken together, the plurality of peptides have corresponding epitope binding activities in samples collectively characteristic of the first and second classes, wherein the autoantibody binding activity of each peptide is independently higher in a sample characteristic of either the first class or the second class as compared to its autoantibody binding activity in a sample characteristic of the other class.

In one embodiment, the invention provides epitope microarrays comprising a plurality of peptides, each peptide having a corresponding epitope binding activity in a first sample or a second sample, wherein the autoantibody binding activity of each peptide is higher or lower with the first sample as compared to the second sample, and wherein the first sample and the second sample correspond to distinct classes.

In a preferred embodiment, at least a first peptide of the epitope microarray has higher autoantibody binding activity with a first sample corresponding to a first class as compared to its autoantibody binding activity with a second sample corresponding to a second class, and at least a second peptide of the epitope microarray has higher autoantibody binding activity with the second sample corresponding to the second class as compared to its autoantibody binding activity with the first sample corresponding to the first class.

Each peptide included on an epitope microarray displays an autoantibody binding activity that correlates with a class distinction, though the frequency at which autoantibody binding activity for any particular epitope is detected may be low, and the probability of detecting a particular epitope-binding autoantibody in a sample characteristic of a particular class may be low. Such epitopes are nonetheless useful for diagnosis when used in combination, as disclosed herein.

Preferred distinct classes include a non-disease class and a disease class, more preferably a non-cancer class and a cancer class, the latter preferably being lung cancer, breast cancer, gastrointestinal cancer, or prostate cancer. Other preferred distinct classes are a high risk class and a non-disease class, preferably a high risk cancer class and a non-cancer class. Other preferred distinct classes are distinct cancer classes, such as distinct lung cancer classes, such as NSCLC and SCLC. Other preferred distinct cancer classes are metastatic cancer and non-metastatic cancer classes.

In a preferred embodiment, two or more peptides of the epitope microarray correspond to distinct regions of a single protein, preferably non-overlapping regions of the single protein.

As disclosed herein, epitopes corresponding to different segments of a single protein can exhibit discordant differences in their binding activities between samples from different classes. Without being bound by theory, this discordance of autoantibody binding activities between epitopes corresponding to the same protein may be due, in part, to protein alterations and consequent epitope alterations that contribute to the distinction of the classes. In support, splice variants of a large number of mRNAs, including mRNAs encoding embryonal transcription factors, have been identified in a variety of cancers.

In one embodiment, one or more peptides of the array is directed to an autoantibody that specifically binds the protein product of an alternatively spliced mRNA that is present or predominant, with respect to transcripts of the particular gene, in a first class, but absent or nondominant in a second class.

At least a first peptide of an epitope microarray herein has higher autoantibody binding activity with a first sample corresponding to a first class as compared to its autoantibody binding activity with a second sample corresponding to a second class, and at least a second peptide of the epitope microarray has higher autoantibody binding activity with the second sample corresponding to the second class as compared to its autoantibody binding activity with the first sample corresponding to the first class. Thus between two distinct classes, autoantibody binding activity that is higher in each class detectable with the preferred microarrays of the invention. With respect to cancer diagnostics, the preferred cancer diagnostic microarrays include epitopes capable of detecting autoantibody binding activities that are higher in a non-cancer sample than a cancer sample, as well as epitopes that are capable of detecting autoantibody binding activities that are higher in a cancer sample than a non-cancer sample, the latter potentially attributable to the appearance of tumor-associated antigens in an individual with cancer.

Once binding of autoantibody to array-bound epitope, and binding of detection agent to immobilized autoantibody occurs, the arrays are inserted into a scanner which can detect patterns of binding. The autoantibody binding data may be collected as light emitted from the labeled groups of the detection agents bound to the array. Since the position of each epitope on the array is known, particular autoantibody binding activities are determined. The amount of light detected by the scanner becomes raw data that the invention applies and utilizes. The epitope array is only one example of obtaining the raw autoantibody binding activity data. Other methods for determining autoantibody binding activity known in the art (eg., ELISA, phage display, etc.), or developed in the future can be used with the present invention.

### Peptide Epitopes and Microarray Preparation

Peptides, as used herein, includes modified peptides, such as phosphopeptides. Peptides may be derived from any of a number of sources, as appreciated by one of skill in the art. For example, random peptides may be generated by expression systems known in the art. Peptides may be generated by extensive protein fragmentation. Preferably, peptides are synthesized according to methods well known in the art. For example, see Methods in Enzymology, Volume 289: Solid-Phase Peptide Synthesis, J. Abelson et al., Academic Press, 1st edition, Nov. 15, 1997, ISBN 0121821900. In a preferred embodiment, a Perkin-Elmer Applied Biosystems 433A Peptide synthesizer is used to synthesize peptides, allowing for synthesis of modified peptides.

Epitope microarrays may be prepared according to methods well known in the art. For example, see Protein Microarray Technology, D. Kambhampati (ed.), John Wiley & Sons, Mar. 5, 2004, ISBN 3527305971; Protein Microarrays, M. Schena, Jones & Bartlett Publishers, July, 2004, ISBN 0763731277; and Protein Arrays: Methods and Protocols (Methods in Molecular Biology), E. Fung, Humana Press, Apr. 1, 2004, ISBN 158829255X. In a preferred embodiment, a Piezorray Non-contact Spotting System from Perkin Elmer is used according to the manufacturer's specifications.

### Sample Sources and Manipulation

A sample can be any sample comprising autoantibodies. Preferred samples include blood, plasma, cerebrospinal fluid, and synovial fluid.

Blood may be collected from each individual by venipuncture. 0.1-0.5 ml may be used to prepare blood serum or plasma. Serum may be prepared just after blood drawing. Tubes may be left at room temperature for 4 hours following centrifugation at 170×g for 5 minutes after which serum is removed. Serum may be aliquoted and stored at −20° C. Plasma may be prepared by adding EDTA (final concentration of 5 mM) to blood sample. Blood sample may be centrifuged at 170×g for 5 minutes, supernatant removed and stored at −20° C.

TABLE 1
Informative Epitopes - Disclosed are 1,448 peptide epitopes, as well as
corresponding protein names, Genbank accession numbers, and peptide sites. These epitopes may
be used as an initial set for autoantibody profiling. Of these, 1,253 were used as an initial set to
measure autoantibody binding activities in lung cancer samples. See Experimental.

| Gene | Accession # | position | epitope | length |
|---|---|---|---|---|
| ACADVL - acyl-Coenzyme A | NM_000018 | | | |
| dehydrogenase, very long chain | | | | |
| ACADVL745 | | 745 | KHKKGIVNEQFLLQ | 14 |
| ACADVL860 | | 860 | WQQELYRNFKSISKA | 15 |
| ACADVL407 | | 407 | KMGIKASNTAEVFFD | 15 |
| ACADVL324 | | 324 | CGKYYTLNGSKLWIS | 15 |
| ACADVL487 | | 487 | KAVDHATNRTQFGEK | 15 |
| ACADVL257 | | 257 | LFGTKAQKEKYLPKL | 15 |
| ACADVL661 | | 661 | ALKNPFGNAGLLLGE | 15 |
| ADSL - adenylosuccinate lyase | NM_000026 | | | |
| ADSL244 | | 244 | DLCMDLQNLKRVRDD | 15 |
| ADSL85 | | 85 | QIQEMKSNLENIDFK | 15 |
| ADSL164 | | 164 | TDLIILRNALDLLLP | 15 |
| ADSL156 | | 156 | TSCYVGDNTDLIILR | 15 |
| ADSL476 | | 476 | TADTILNTLQNISEG | 15 |
| ADSL411 | | 411 | RCCSLARHLMTLVMD | 15 |
| ADSL97 | | 97 | DFKMAAEEEKRLRHD | 15 |
| AP1G2 - adaptor-related protein | NM_003917 | | | |
| complex 1, gamma 2 subunit | | | | |
| AP1G2584 | | 584 | VRDDAVANLTQLIGG | 15 |
| AP1G2497 | | 497 | ELSLALVNSSNVRAM | 15 |
| AP1G2500 | | 500 | LALVNSSNVRAMMQE | 15 |
| AP1G2425 | | 425 | FLLNSDRNIRYVALT | 15 |
| AP1G21020 | | 1020 | LFRILNPNKAPLRLK | 15 |
| AP1G2656 | | 656 | GDLLLAGNCEEIEPL | 15 |
| AP1G2938 | | 938 | SFIRPPENPALLLIT | 15 |
| AP1G2701 | | 701 | LLEKVLQSHMSLPAT | 15 |
| AP1G2967 | | 967 | ICQAAVPKSLQLQLQ | 15 |
| AP1G2388 | | 388 | DTSRNAGNAVLFETV | 15 |
| ASCC3L1 - activating signal | NM_014014 | | | |
| cointegrator 1 complex subunit 3-like 1 | | | | |
| ASCC3L1884 | | 884 | GLSATLPNYEDVATF | 15 |
| ASCC3L12395 | | 2395 | RRMTQNPNYYNLQGI | 15 |
| ASCC3L11965 | | 1965 | RRWKQRKNVQNINLF | 15 |
| ASCC3L12472 | | 2472 | IAAYYYINYTTIELF | 15 |
| ASCC3L1405 | | 405 | SDDRECENQLVLLLG | 15 |
| ASCC3L11968 | | 1968 | KQRKNVQNINLFVVD | 15 |
| ASCC3L12519 | | 2519 | GLIEIISNAAEYENI | 15 |
| ASCC3L1659 | | 659 | LYRAALETDENLLLC | 15 |
| BAIAP3 - BAI1-associated protein 3 | NM_003933 | | | |
| BAIAP31198 | | 1198 | LSPDSIQNDEAVAPL | 15 |
| BAIAP31099 | | 1099 | ALCVVLNNVELVRKA | 15 |
| BAIAP31217 | | 1217 | DEKLALLNASLVVRK | 15 |
| BAIAP3567 | | 567 | EHSAEEPNSSSWRGE | 15 |
| BOP1 - block of proliferation 1 | NM_015201 | | | |
| BOP1641 | | 641 | LVAAAVEDSVLLLNP | 15 |
| BOP1825 | | 825 | LTKKLMPNCKWVS | 13 |
| Cep290 - Homo sapiens centrosome | NM_025114 | | | |
| protein cep290 (Cep290), mRNA. | | | | |
| Cep290707 | | 707 | IDLTEFRNSKHLKQQ | 15 |
| Cep2901287 | | 1287 | ALQKVVDNSVSLSEL | 15 |
| Cep2901345 | | 1345 | MLVQRTSNLEHLECE | 15 |
| Cep2901423 | | 1423 | KAKKSITNSDIVSIS | 15 |
| Cep2903023 | | 3023 | KLRIAKNNLEILNEK | 15 |
| Cep290471 | | 471 | QLDADKSNVMALQQG | 15 |
| Cep2902537 | | 2537 | QGKPLTDNKQSLIEE | 15 |
| Cep2902465 | | 2465 | RENSLTDNLNDLNNE | 15 |
| Cep2901107 | | 1107 | RKFAVIRHQQSLLYK | 15 |
| CGI-09 - Homo sapiens CGI-09 protein | NM_015939 | | | |
| (CGI-09), mRNA. | | | | |
| CGI-09637 | | 637 | ADTSLKSNASTLESH | 15 |
| CGI-09169 | | 169 | IVQQLIENSTTFRDK | 15 |
| CGI-09575 | | 575 | LSETWLRNYQVLPDR | 15 |
| CGI-09490 | | 490 | AALLSERNADGLIVA | 15 |
| CGI-0987 | | 87 | GTAFEVTSGGSLQPK | 15 |
| CGI-63 - Homo sapiens nuclear | NM_016011 | | | |
| receptor binding factor 1 (CGI-63) | | | | |
| CGI-63100 | | 100 | KMLAAPINPSDINMI | 15 |
| CGI-63156 | | 156 | QVVAVGSNVTGLKPG | 15 |
| CHTF18 - CTF18, chromosome | NM_022092 | | | |
| transmission fidelity factor 18 homolog | | | | |
| CHTF181110 | | 1110 | YIYRLEPNVEELCRF | 15 |
| CHTF18882 | | 882 | VVQGLFDNFLRLRLR | 15 |
| CLK3 - CDC-like kinase 3 | NM_001292 | | | |
| CLK3158 | | 158 | RRTRSCSSASSMRLW | 15 |
| COTL1 - coactosin-like 1 | NM_021149 | | | |
| COTL1154 | | 154 | AKEFVISDRKELEED | 15 |
| CSDA | | | | |
| CSDA - cold shock domain protein A | NM_003651 | | | |
| CSDA422 | | 422 | QQATSGPNQPSVRRG | 15 |
| CSDA7 | | 7 | AGEATTTTTTTLPQA | 15 |
| CSDA175 | | 175 | PQARSVGDGETVEFD | 15 |
| DKFZp434F054 - Homo sapiens | NM_032259 | | | |
| hypothetical protein DKFZp434F054 | | | | |
| DKFZp434F054-113 | | 113 | LLATAATNGVVVTW | 14 |
| DKFZp434F054-650 | | 650 | LPLMNSFNLKDMAPG | 15 |
| DKFZp434F054-647 | | 647 | SCGLPLMNSFNLKDM | 15 |
| DKFZp434F054-26 | | 26 | CHLDAPANAISVCRD | 15 |
| DKFZp434F054-701 | | 701 | SDTVLLDSSATLITN | 15 |
| EEF1D - eukaryotic translation | NM_001960 | | | |
| elongation factor 1 delta | | | | |
| EEF1D-37 | | 37 | AGASRQENGAS | 11 |
| EFHD2 - EF hand domain containing 2 | NM_024329 | | | |
| EFHD2-113 | | 113 | FSRKQIKDMEKMFK | 14 |
| EXOSC9 - exosome component 9 | NM_005033 | | | |
| EXOSC9-246 | | 246 | LILKALENDQKVRKE | 15 |
| EXOSC9-24 | | 24 | LMERCLRNSKCIDTE | 15 |
| FAHD1 - fumarylacetoacetate hydrolase | NM_031208 | | | |
| domain containing 1 | | | | |
| FAHD1-104 | | 104 | KRCRAVPEAAAMDYV | 15 |
| FAHD1-36 | | 36 | EMRSAVLSEPVL | 12 |
| FAHD1-237 | | 237 | YIISYVSKIITLEEG | 15 |
| FLJ10385 - Homo sapiens hypothetical | NM_018081 | | | |
| protein FLJ10385 | | | | |
| ELJ10385-629 | | 629 | LPQKDCTNGVSLHPS | 15 |
| ELJ10385-332 | | 332 | VASSSRENPIHIWDA | 15 |
| ELJ10385-250 | | 250 | ILTNSADNILRIYNL | 15 |
| FLJ10385-157 | | 157 | SLSEEEANGPELGSG | 15 |
| FLJ10385-556 | | 556 | SLGREVTTNQRIYFD | 15 |
| FLJ10385-247 | | 247 | GSCILTNSADNILRI | 15 |
| ELJ10385-578 | | 578 | LVSGSTSGAVSVWDT | 15 |
| ELJ10385-557 | | 557 | LGREVTTNQRIYFDL | 15 |
| FLJ10385-321 | | 321 | LMSSAQPDTSYVASS | 15 |
| GL009 - Homo sapiens hypothetical | NM_032492 | | | |
| protein GL009 | | | | |
| GL009-113 | | 113 | LLSFPRNNISYLVL | 14 |
| GL009-184 | | 184 | LFGFSAVSIMYLVLV | 15 |
| GL009-76 | | 76 | VAKMSVGHLRLLSHD | 15 |
| GL009-15 | | 15 | TDGSDFQHRERVAMH | 15 |
| GNPTAG - N-acetylglucosamine-1- | NM_032520 | | | |
| phosphotransferase, gamma subunit | | | | |
| GNPTAG-379 | | 379 | SNLEHL | 12 |
| GNPTAG-263 | | 263 | DELITPQGHEKLLRT | 15 |
| GNPTAG-109 | | 109 | PFHNVTQHEQTFRWN | 15 |
| GRINA - glutamate receptor, ionotropic, | XM_291268 | | | |
| GRINA-299 | | 299 | NTEAVIMA | 8 |
| GRINA-255 | | 255 | FRRKHPWNLVALSVL | 15 |
| GRINA-421 | | 421 | YVFAALNLYTDIINI | 15 |
| GRINA-224 | | 224 | FVRENVWTYYVS | 12 |
| GRINA-398 | | 398 | TCFLAVDTQLLLGNK | 15 |
| GTF2H2 - general transcription factor | NM_001515 | | | |
| IIH, polypeptide 2 | | | | |
| GTF2H2-240 | | 240 | LTTCDPSNIYDLIKT | 15 |
| GTF2H2-185 | | 185 | HGEPSLYNSLSIAMQ | 15 |
| GTF2H2-325 | | 325 | PPPASSSSECSLIRM | 15 |
| GTF2H2-487 | | 487 | YVCAVCQNVFCVDCD | 15 |
| GTF2H2-151 | | 151 | IIVTKSKRAEKLTEL | 15 |
| GTF2H2-193 | | 193 | SLSIAMQTLKHMP | 13 |
| GTF2H2-462 | | 462 | PLEEYNGERFCYG | 13 |
| HAGH - hydroxyacylglutathione | NM_005326 | | | |
| hydrolase | | | | |
| HAGH-8 | | 8 | VLPALTDNYMYLVID | 15 |
| HAGH-238 | | 238 | GHEYTINNLKFARHV | 15 |
| HAGH-108 | | 108 | ALTHKITHLSTLQVG | 15 |
| HAGH-80 | | 80 | HWDHAGGNEKLVKLE | 15 |
| HAGH-105 | | 105 | RIGALTHKITHLSTL | 15 |
| HAGHL - hydroxyacylglutathione | NM_032304 | | | |
| hydrolase-like | | | | |
| HAGHL-8 | | 8 | VIPVLEDNYMYLVIE | 15 |
| HAGHL-237 | | 237 | GHEHTLSNLEFAQKV | 15 |
| HAGHL-190 | | 190 | LEGSAQQMYQSLAEL | 15 |
| HAGHL-193 | | 193 | SAQQMYQSLAELG | 13 |
| HAGHL-108 | | 108 | SLTRRLAHGEELRFG | 15 |
| HDAC5 - histone deacetylase 5 | NM_005474 | | | |
| HDAC5-1027 | | 1027 | LYGTSPLNRQKLDSK | 15 |
| HDAC5-481 | | 481 | LPLDSSPNQFSLYTS | 15 |
| HDAC5-1194 | | 1194 | GTQQAFYNDPSVLYI | 15 |
| HDAC5-1112 | | 1112 | VAAGELKNGFAIIRP | 15 |
| HDAC5-102 | | 102 | QELLALKQQQQLQKQ | 15 |
| HDAC5-1136 | | 1136 | AMGFCFFNSVAITAK | 15 |
| HDAC5-1414 | | 1414 | AVLQQKPNINAVATL | 15 |
| HDAC5-702 | | 702 | QLVMQQQHQQFL | 15 |
| HDAC5-175 | | 175 | QEMLAAKRQQELEQQ | 15 |
| HDAC5-506 | | 506 | QATVTVTNSHLTASP | 15 |
| HDAC5-426 | | 426 | GPSSPNSSHSTIAEN | 15 |
| HDAC5-487 | | 487 | PNQFSLYTSPSLPNI | 15 |
| HDAC5-644 | | 644 | TGERVATSMRTVGKL | 15 |
| HLA-B - major histocompatibility | NM_005514 | | | |
| complex, class I, B | | | | |
| HLA-B-115 | | 115 | YKAQAQTDRESL | 12 |
| HLA-B-182 | | 182 | HDQYAYDGKDYIALN | 15 |
| HLA-C - major histocompatibility | NM_002117 | | | |
| complex, class I, C | | | | |
| HLA-C-479 | | 479 | CSNSAQGSDESLITC | 15 |
| HLA-C-182 | | 182 | YDQSAYDGKDYIALN | 15 |
| HLA-C-258 | | 258 | LRRYLENGKETLQRA | 15 |
| HSPA4 - heat shock 70 kDa protein 4 | NM_002154 | | | |
| HSPA4-1022 | | 1022 | NNKLNLQNKQSLTMD | 15 |
| HSPA4-381 | | 381 | MSANASDLPLS | 12 |
| HSPA4-76 | | 76 | AKSQVISNAKNTVQG | 15 |
| HSPA4-873 | | 873 | FVSEDDRNSFTLKLE | 15 |
| HSPA4-1016 | | 1016 | AMEWMNNKLNLQNK | 14 |
| HSPA4-966 | | 966 | KIISSFKNKEDQYDH | 15 |
| HSPA4-806 | | 806 | MLNLYIENEGKMIMQ | 15 |
| HSPA4-658 | | 658 | HGIFSVSSASLVEVH | 15 |
| HSPH1 - heat shock 105 kDa/110 kDa | NM_006644 | | | |
| protein 1 | | | | |
| HSPH1-381 | | 381 | MSSNSTDLPLN | 12 |
| HSPH1-83 | | 83 | HANNTVSNFKRFHGR | 15 |
| HSPH1-891 | | 891 | ICEQDHQNFLRLLTE | 15 |
| HSPH1-780 | | 780 | IPDADKANEKKVDQP | 15 |
| HSPH1-71 | | 71 | TIGVAAKNQQITHAN | 15 |
| HSPH1-1141 | | 1141 | ECYPNEKNSVNMD | 13 |
| HSPH1-1107 | | 1107 | PKLERTPNGPNIDKK | 15 |
| IQWD1 - IQ motif and WD repeats 1 | | | | |
| IQWD1-173 | | 173 | LDEQQDNNNEKLSPK | 15 |
| IQWD1-315 | | 315 | SAENPVENHINITQS | 15 |
| IQWD1-655 | | 655 | LMLEETRNTITVPAS | 15 |
| IQWD1-28 | | 28 | RGGTSQSDISTLPTV | 15 |
| IQWD1-338 | | 338 | DSNSGERNDLNLDRS | 15 |
| IQWD1-646 | | 646 | ADEVITRNELMLEET | 15 |
| IQWD1-395 | | 395 | TSTESATNENNTNPE | 15 |
| JPH4 - junctophilin 4 | NM_032452 | | | |
| JPH4-498 | | 498 | RAVSAARQRQEIAAA | 15 |
| KIAA0373/centrosome protein cep290 | NM_025114 | | | |
| KIAA0373-707 | | 707 | IDLTEFRNSKHLKQQ | 15 |
| KIAA0373-1287 | | 1287 | ALQKVVDNSVSLSEL | 15 |
| KIAA0373-1345 | | 1345 | MLVQRTSNLEHLECE | 15 |
| KIAA0373-1410 | | 1410 | ETKLGNESSMDKA | 13 |
| KIAA0373-1423 | | 1423 | KAKKSITNSDIVSIS | 15 |
| KIAA0373-3203 | | 3203 | KLRIAKNNLEILNEK | 15 |
| KIAA0373-271 | | 271 | RSQLSKKNYELIQY | 14 |
| KIAA0373-471 | | 471 | QLDADKSNVMALQQG | 15 |
| KIAA0373-113 | | 113 | TKVMKLENELEMAQ | 14 |
| KIAA0373-2537 | | 2537 | QGKPLTDNKQSLIEE | 15 |
| KIAA0373-2465 | | 2465 | RENSLTDNLNDLNNE | 15 |
| KIAA0373-938 | | 938 | VNAIESKNAEGIFDA | 15 |
| KIAA0373-1107 | | 1107 | RKFAVIRHQQSLLYK | 15 |
| KIAA0373-807 | | 807 | LDLLSLKNMSEAQSK | 15 |
| KIAA0373-634 | | 634 | VEIKNCKNQIKIRDR | 15 |
| KIAA0373-2401 | | 2401 | SQKEAHLNVQQIVDR | 15 |
| KIAA0373-1203 | | 1203 | KITVLQVNEKSLIRQ | 15 |
| KIAA0373-1193 | | 1193 | MKKILAENSRKITVL | 15 |
| KIAA0373-720 | | 720 | QQQYRAENQILLKEI | 15 |
| KIAA0373-3110 | | 3110 | KKNQSITDLKQLVKE | 15 |
| KIAA0373-2294 | | 2294 | KVKAEVEDLKYLLDQ | 15 |
| KIAA0373-1050 | | 1050 | ASIINSQNEYLIHLL | 15 |
| KIAA0373-64 | | 64 | QENVIHLFRI | 10 |
| KIAA0373-2692 | | 2692 | LGIRALESEKELEEL | 15 |
| KIAA0373-1972 | | 1972 | DPSLPLPNQLEIALR | 15 |
| KIAA0373-3234 | | 3234 | GAESTIPDADQLKEK | 15 |
| KIAA0373-1210 | | 1210 | NEKSLIRQYTTLVEL | 15 |
| KIAA0683 | NM_016111 | | | |
| KIAA0683-234 | | 234 | GNRLQQENLAEFFPQ | 15 |
| KIAA0683-242 | | 242 | LAEFFPQNYFRLLGE | 15 |
| KIAA0683-868 | | 868 | QPGSPSPNTPCLPEA | 15 |
| KIAA0683-323 | | 323 | PRLAALTQGSYLHQR | 15 |
| KRT18 - keratin 18 | NM_000224 | | | |
| KRT18-8 | | 8 | TRSTFSTNYRSLGSV | 15 |
| KRT18-343 | | 343 | YDELARKNREELDKY | 15 |
| KRT18-185 | | 185 | IFANTVDNARIVLQI | 15 |
| KRT18-566 | | 566 | GKVVSETNDTKVLRH | 15 |
| KRT18-544 | | 544 | DALDSSNSMQTIQKT | 15 |
| KRT18-252 | | 252 | RKVIDDTNITRLQLE | 15 |
| KRT18-567 | | 567 | KVVSETNDTKVLRH | 14 |
| KRT18-484 | | 484 | EGQRQAQEYEALLNI | 15 |
| KRT18-96 | | 96 | AGMGGIQNEKETMQS | 15 |
| LDHB - lactate dehydrogenase B | NM_002300 | | | |
| LDHB-347 | | 347 | LIESMLKNLSRIHPV | 15 |
| LDHB-18 | | 18 | EEATVPNNKITVVGV | 15 |
| LDHB-387 | | 387 | KGMYGIENEVFLSLP | 15 |
| LDHB-177 | | 177 | CIIIVVSNPVDILTY | 15 |
| LDHB-106 | | 106 | KDYSVTANSKIVVVT | 15 |
| LDHB-307 | | 307 | GTDNDSENWKEVHKM | 15 |
| LDHB-17 | | 17 | EEEATVPNNKITVVG | 15 |
| LGALS4 - lectin, galactoside-binding, | NM_006149 | | | |
| soluble, 4 (galectin 4) | | | | |
| LGALS4-391 | | 391 | DRFKVYANGQHLFDF | 15 |
| LGALS4-237 | | 237 | HCHQQLNSLPTMEGP | 15 |
| LGALS4-407 | | 407 | HRLSAFQRVDTLEIQ | 15 |
| LGALS4-415 | | 415 | VDTLEIQGDVTLSYV | 15 |
| LGALS4-155 | | 155 | EHYKVVVNGNPFYEY | 15 |
| LOC162962 - similar to zinc finger | XM_091886 | | | |
| protein 616 | | | | |
| LOC162962-177 | | 177 | VENKCIENQLTLSFQ | 15 |
| LOC162962-232 | | 232 | QSEKTVNNSSLVSPL | 15 |
| LOC162962-36 | | 36 | YWDVMLENYRNL | 12 |
| LOC162962-497 | | 497 | RQNSNLVNHQRIHTG | 15 |
| LOC162962-315 | | 315 | RVSSSLINHQMVHTT | 15 |
| LOC162962-854 | | 854 | LSNHKRIHTG | 10 |
| LOC162962-799 | | 799 | ECGTVFRNYSCLARH | 15 |
| LOC162962-1113 | | 1113 | RVRSILVNHQKMHTG | 15 |
| LOC162962-231 | | 231 | NQSEKTVNNSSLVSP | 15 |
| LOC162962-111 | | 111 | YLREIQKNLQDLEFQ | 15 |
| LOC162962-1189 | | 1189 | FGRFSCLNKHQMIHS | 15 |
| LOC162962-543 | | 543 | KSFSQSSNLATHQTV | 15 |
| LOC162962-904 | | 904 | DCGKAYTQRSSLT | 13 |
| LOC388198- | XM_373655 | | | |
| LOC388198-145 | | 145 | RSSTGAYALRLC | 12 |
| LOC388198-9 | | 9 | GAAYSAQRMAGLVLP | 15 |
| LOC388561 - similar to zinc finger | XM_371192 | | | |
| protein 600 | | | | |
| LOC388561-230 | | 230 | NESGKAFNYSSLLRK | 15 |
| LOC388561-182 | | 182 | NHGNNFWNSSLLTQK | 15 |
| LOC388561-7 | | 7 | FLSTAQGNREVFHAG | 15 |
| LOC388561-461 | | 461 | KTFSHKSSLTCH | 12 |
| LOC388561-412 | | 412 | ECGKTFSHKSSLTCH | 15 |
| LOC388561-307 | | 307 | ECGKTFSQTSSLTCH | 15 |
| LOC388561-874 | | 874 | ECGKNFSQKSSLICH | 15 |
| LOC401193 - similar to psi neuronal | XM_376391 | | | |
| apoptosis inhibitory protein | | | | |
| LOC401193-87 | | 87 | NTASSSLNIFSLLPT | 15 |
| LOC401193-77 | | 77 | KEPISLNNSINTASS | 15 |
| LOC401193-156 | | 156 | EFLRSKKSSEEITQY | 15 |
| LOC90333 | XM_030958 | | | |
| LOC90333-12 | | 12 | IQSFKSFNCSSLLKK | 15 |
| LOC90333-398 | | 398 | ECGKTFSQMSSLVYH | 15 |
| LOC90333-321 | | 321 | VCDKAFQRDSHLAQH | 15 |
| LSM1 - LSM1 homolog, U6 small | NM_014462 | | | |
| nuclear RNA associated | | | | |
| LSM1-164 | | 164 | DRGLSIPRADTLDEY | 15 |
| LSM1-33 | | 33 | GFLRSIDQFANLVLH | 15 |
| LSM1-87 | | 87 | IFVVRGENVVLLGEI | 15 |
| MAGEA4 - melanoma antigen, family A, 4 | NM_002362 | | | |
| MAGEA4-234 | | 234 | KEVDPTSNTYTLVTC | 15 |
| MAGEA4-181 | | 181 | MLERVIKNYKRCFPV | 15 |
| MAGEA4-85 | | 85 | GPPQSPQGASALPTT | 15 |
| MIF - macrophage migration inhibitory | NM_002415 | | | |
| factor | | | | |
| MIF-141 | | 141 | NAANVGWN | 8 |
| MIF-92 | | 92 | IGGAQNRSYSKLLCG | 15 |
| MIF-115 | | 115 | SPDRVYINYYDM | 12 |
| MSLN - mesothelin | NM_005823 | | | |
| MSLN-74 | | 74 | GVLANPPNISSLSPR | 15 |
| MSLN-71 | | 71 | PLDGVLANPPNISSL | 15 |
| MSLN-186 | | 186 | FSRITKANVDLLPRG | 15 |
| MSLN-652 | | 652 | RLAFQNMNGSEYFVK | 15 |
| MSLN-510 | | 510 | PEDIRKWNVTSL | 12 |
| MSLN-324 | | 324 | PSTWSVSTMDALRGL | 15 |
| MSLN-259 | | 259 | PGRFVAESAEVLLPR | 15 |
| NACA - nascent-polypeptide-associated | NM_005594 | | | |
| complex alpha | | | | |
| NACA-261 | | 261 | AVRALKNNSNDIVNA | 15 |
| NACA-66 | | 66 | QATTQQAQLAAA | 12 |
| NACA-251 | | 251 | MSQANVSRAKAVRAL | 15 |
| NISCH - nischarin | NM_007184 | | | |
| NISCH-428 | | 428 | NGLLVVDNLQHLYNL | 15 |
| NISCH-478 | | 478 | GLHTKLGNIKTLNLA | 15 |
| NISCH-805 | | 805 | CIGYTATNQDFIQRL | 15 |
| NISCH-1764 | | 1764 | KTTGKMENYELIHSS | 15 |
| NISCH-555 | | 555 | EHVSLLNNPLSIIPD | 15 |
| NISCH-710 | | 710 | ALASSLSSTDSLTPE | 15 |
| NISCH-1271 | | 1271 | THNCRNRNSFKLSRV | 15 |
| NISCH-97 | | 97 | PKKIIGKNSRSLVEK | 15 |
| NISCH-1360 | | 1360 | QLRASLQDLKTVVIA | 15 |
| NISCH-465 | | 465 | HLDLSYNKLSSLEGL | 15 |
| NISCH-333 | | 333 | SVRFSATSMKEVLVP | 15 |
| NISCH-1105 | | 1105 | RSCFAPQHMAMLCSP | 15 |
| NUBP2 - nucleotide binding protein 2 | NM_012225 | | | |
| NUBP2-179 | | 179 | PPGTSDEHMATIEAL | 15 |
| NUBP2-5 | | 5 | EAAAEPGNLAGVRHI | 15 |
| NUBP2-249 | | 249 | RVMGIVENMSGFTCP | 15 |
| OGFR - opioid growth factor receptor | NM_007346 | | | |
| OGFR-165 | | 165 | NYDLLEDNHSYIQWL | 15 |
| OGFR-639 | | 639 | SAAVASGGAQTLALA | 15 |
| OGFR-269 | | 269 | LNWRSHNNLRITRIL | 15 |
| PABPC1 - poly(A) binding protein, | NM_002568 | | | |
| cytoplasmic 1 | | | | |
| PABPC1-796 | | 796 | GMLLEIDNSELLHML | 15 |
| PABPC1-150 | | 150 | NLDKSIDNKALYDTF | 15 |
| PABPC1-90 | | 90 | ERALDTMNFDVIKGK | 15 |
| PABPC1-650 | | 650 | TQRVANTSTQTMGPR | 15 |
| PABPC1-332 | | 332 | QKAVDEMNGKELNGK | 15 |
| PAI-RBP1 - mRNA-binding protein | NM_015640 | | | |
| PAI-RBP1-304 | | 304 | GTVKDELTDLDQS | 13 |
| PAI-RBP1-102 | | 102 | RKNPLPPSVGVVDKK | 15 |
| PAI-RBP1-158 | | 158 | PDQQLQGEGKIIDRR | 15 |
| PDXK - pyridoxal (pyridoxine, vitamin | NM_003681 | | | |
| B6) kinase | | | | |
| PDXK-111 | | 111 | DKSFLAMVVDIVQEL | 15 |
| PDXK-7 | | 7 | ECRVLSIQSHVIRGY | 15 |
| PDXK-114 | | 114 | FLAMVVDIVQELK | 13 |
| PDXK-346 | | 346 | TVSTLHHVLQRTIQC | 15 |
| PDXK-339 | | 339 | LKVACEKTVSTLHHV | 15 |
| PDXK-89 | | 89 | LYEGLRLNNMNKYDY | 15 |
| PDXK-263 | | 263 | NYLIVLGSQRRRNPA | 15 |
| PDXK-101 | | 101 | YDYVLTGYTRDKSFL | 15 |
| RAB40C - member RAS oncogene | NM_021168 | | | |
| family | | | | |
| RAB40C-310 | | 310 | KSFSMANGMNAVMMH | 15 |
| RAB40C-319 | | 319 | NAVMMHGRSYSLASG | 15 |
| RAB40C-225 | | 225 | FNVIESFTELSRI | 13 |
| RAB40C-164 | | 164 | VPRILVGNRLHLAFK | 15 |
| RAB40C-78 | | 78 | TTILLDGRRVRLELW | 15 |
| RAB40C-237 | | 237 | SRIVLMRHGMEKIWR | 15 |
| RAB40C-340 | | 340 | KGNSLKRSKSIRPPQ | 15 |
| RAB40C-334 | | 334 | AGGGGSKGNSLKRSK | 15 |
| RBMS1 - RNA binding motif, single | NM_002897 | | | |
| stranded interacting protein 1 | | | | |
| RBMS1-21 | | 21 | YPQYLQAKQSLVPAH | 15 |
| RBMS1-79 | | 79 | GWDQLSKTNLYIRGL | 15 |
| RBMS1-462 | | 462 | SPLAQQMSHLSLG | 13 |
| RBMS1-157 | | 157 | SPAAAQKAVSALKAS | 15 |
| RBMS1-495 | | 495 | QYAHMQTTAVPVEEA | 15 |
| RBMS1-108 | | 108 | PYGKIVSTKAILDKT | 15 |
| RHBDL1 - rhomboid, veinlet-like 1 | NM_003961 | | | |
| RHBDL1-464 | | 464 | CPYKLLRMVLALVCM | 15 |
| RHBDL1-267 | | 267 | ASVTLAQIIVFLCYG | 15 |
| RHBDL1-349 | | 349 | GFNALLQLMIGVPLE | 15 |
| RHBDL1-503 | | 503 | FMAHLAGAVVGVSMG | 15 |
| RHBDL1-471 | | 471 | MVLALVCMSSEVGRA | 15 |
| RHBDL1-401 | | 401 | LAGSLTVSITDMRAP | 15 |
| RHBDL1-555 | | 555 | WWVVLLAYGTFLLFA | 15 |
| RHBDL1-332 | | 332 | AWRFLTYMFMHVGLE | 15 |
| RHOT2 - ras homolog gene family, | NM_138769 | | | |
| member T2 | | | | |
| RHOT2-309 | | 309 | APQALEDVKTVVCRN | 15 |
| RHOT2-807 | | 807 | LLGVVGAAVAAVLSF | 15 |
| RHOT2-815 | | 815 | VAAVLSFSLYRVLVK | 15 |
| RHOT2-7 | | 7 | DVRILLLGEAQVGKT | 15 |
| RHOT2-335 | | 335 | LDGFLFLNTLFIQRG | 15 |
| RHOT2-543 | | 543 | QAHAITVTREKRLDQ | 15 |
| RHOT2-659 | | 659 | VACLMFDGSDPKSFA | 15 |
| RNPC2 - RNA-binding region (RNP1, | NM_004902 | | | |
| RRM) containing 2 | | | | |
| RNPC2-642 | | 642 | KCPSIAAAIAAVNAL | 15 |
| RNPC2-701 | | 701 | FPDSMTATQLLVPSR | 15 |
| RNPC2-231 | | 231 | RPRDLEEFFSTVGKV | 15 |
| RNPC2-420 | | 420 | NGFELAGRPMKVGHV | 15 |
| RNPC2-662 | | 662 | AGKMITAAYVPLPTY | 15 |
| RNPC2-551 | | 551 | TEASALAAAASVQPL | 15 |
| RNPC2-561 | | 561 | SVQPLATQCFQLSNM | 15 |
| RNPC2-266 | | 266 | EFVDVSSVPLAIGLT | 15 |
| ROCK2 - Rho-associated, coiled-coil | NM_004850 | | | |
| containing protein kinase 2 | | | | |
| ROCK2-1334 | | 1334 | TNRTLTSDVANLANE | 15 |
| ROCK2-403 | | 403 | YADSLVGTYSKIMDH | 15 |
| ROCK2-1517 | | 1517 | DIEQLRSQLQALHIG | 15 |
| ROCK2-163 | | 163 | YAMKLLSKFEMIKRS | 15 |
| ROCK2-66 | | 66 | SLLDGLNSLVLD | 12 |
| ROCK2-1127 | | 1127 | ENNHLMEMKMNLEKQ | 15 |
| ROCK2-1018 | | 1018 | EERTLKQKVENLLLE | 15 |
| ROCK2-1296 | | 1296 | HKQELTEKDATIASL | 15 |
| ROCK2-644 | | 644 | VNTRLEKTAKELEEE | 15 |
| ROCK2-818 | | 818 | KNCLLETAKLKLEKE | 15 |
| RPL15- ribosomal protein L15 | NM_002948 | | | |
| RPL15-118 | | 118 | FARSLQSVA | 9 |
| RPL15-114 | | 114 | NQLKFARSLQSVA | 12 |
| RPL15-17 | | 17 | KQSDVMRFLLRVRCW | 15 |
| RUNDC1 - RUN domain containing 1 | NM_173079 | | | |
| RUNDC1-704 | | 704 | PKQSLLTAIHMVLTE | 15 |
| RUNDC1-795 | | 795 | SALNLLSRLSSLKFS | 15 |
| RUNDC1-110 | | 110 | ERRRLDSALLALSSH | 15 |
| RUNDC1-466 | | 466 | TGLHLMRRALAVLQI | 15 |
| RUNDC1-439 | | 439 | NEQRLVSWVNLICKS | 15 |
| RUNDC1-316 | | 316 | LDMNLNEDISSLSTE | 15 |
| RUNDC1-507 | | 507 | YSPLLKRLEVSVDRV | 15 |
| RUNDC1-332 | | 332 | LRQRVDAAVAQIVNP | 15 |
| RUNDC1-248 | | 248 | QKELILQLKTQLDDL | 15 |
| RUNDC1-3 | | 3 | MAAIEAAAEPVTVV | 15 |
| RUNDC1-576 | | 576 | VRKELTVAVRDLLAH | 15 |
| RUTBC3 - RUN and TBC1 domain | NM_015705 | | | |
| containing 3 | | | | |
| RUTBC3-862 | | 862 | PEELLYRAVQSVNVT | 15 |
| RUTBC3-386 | | 386 | LHWFLTAFASVVDIK | 15 |
| RUTBC3-904 | | 904 | WLEVLCSSLPTVE | 13 |
| RUTBC3-482 | | 482 | VAMRLAGSLTDVAVE | 15 |
| RUTBC3-475 | | 475 | DAELLLGVAMRLAGS | 15 |
| RUTBC3-581 | | 581 | LVADLREAILRVARH | 15 |
| RUTBC3-892 | | 892 | ICVGLNEQVLHLWLE | 15 |
| RUTBC3-462 | | 462 | NTLSDIPSQMEDA | 13 |
| RUTBC3-81 | | 81 | PGSSLLANSPLMEDA | 15 |
| RUTBC3-307 | | 307 | AFWMMSAIIEDLLPA | 15 |
| RUTBC3-246 | | 246 | GVPRLRRVLRALAWL | 15 |
| RUTBC3-413 | | 413 | GSRVLFQLTLGMLHL | 15 |
| RUTBC3-338 | | 338 | LRHLIVQYLPRLDKL | 15 |
| RUTBC3-740 | | 740 | GDDSVTEGVTDLVRG | 15 |
| RUTBC3-349 | | 349 | LDKLLQEHDIELSLI | 15 |
| RUTBC3-502 | | 502 | HLAYLIADQGQLLGA | 15 |
| SBDS - Shwachman-Bodian-Diamond | NM_016038 | | | |
| syndrome | | | | |
| SBDS-71 | | 71 | LDEVLQTHSVFVNVS | 15 |
| SBDS-108 | | 108 | CKQILTKGEVQVSDK | 15 |
| SBDS-252 | | 252 | LKEKLKPLIKVIESE | 15 |
| SBDS-148 | | 148 | QLEQMFRDIATIVAD | 15 |
| SCNN1A - sodium channel, nonvoltage- | NM_001038 | | | |
| gated 1 alpha | | | | |
| SCNN1A-732 | | 732 | PSVTMVTLLSNLGSQ | 15 |
| SCNN1A-346 | | 346 | ILSRLPETLPSLEED | 15 |
| SCNN1A-786 | | 786 | VFDLLVIMFLMLLRR | 15 |
| SCNN1A-343 | | 343 | YINILSRLPETLPSL | 15 |
| SCNN1A-88 | | 88 | NNTTIHGAIRLVCSQ | 15 |
| SCNN1A-272 | | 272 | VASSLRDNNPQVD | 13 |
| SCNN1A-166 | | 166 | NSDKLVFPAVTICTL | 15 |
| SCNN1A-778 | | 778 | VEMAELVFDLLVI | 13 |
| SCNN1A-471 | | 471 | LLSTVTGARVMVHGQ | 15 |
| SCNN1A-787 | | 787 | FDLLVIMFLMLLRRF | 15 |
| SCNN1A-502 | | 502 | VETSISMRKETLDRL | 15 |
| SCNN1A-745 | | 745 | SQWSLWFGSSVLSV | 14 |
| SCNN1A-226 | | 226 | LYKYSSFTTLVAGS | 14 |
| SCNN1A-184 | | 184 | RYPEIKEELEELDRI | 15 |
| SCP2 - sterol carrier protein 2 | NM_002979 | | | |
| SCP2-330 | | 330 | QKYGLQSKAVEILAQ | 15 |
| SCP2-318 | | 318 | AAAAILASEAFVQKY | 15 |
| SCP2-719 | | 719 | GNMGLAMKLQNLQLQ | 15 |
| SCP2-728 | | 728 | QNLQLQPGNAKL | 13 |
| SCP2-165 | | 165 | GFEKMSKGSLGIKFS | 15 |
| SCP2-418 | | 418 | TNELLTYEALGLCPE | 15 |
| SCP2-153 | | 153 | IQGGVAECVLALGFE | 16 |
| SCP2-268 | | 268 | DEYSLDEVMASKEVF | 15 |
| SCP2-233 | | 233 | GKEHMEKYGTKIEHF | 15 |
| SCP2-100 | | 100 | IYHSLGMTGIPIINV | 15 |
| SDCCAG1 - serologically defined colon | NM_004713 | | | |
| cancer antigen 1, NY-CO-1 | | | | |
| SDCCAG1-13 | | 13 | LRAVLAELNASLLGM | 15 |
| SDCCAG1-934 | | 934 | LASCTSELISE | 13 |
| SDCCAG1-232 | | 232 | TLERLTEIVASAPKG | 15 |
| SDCCAG1-860 | | 860 | TGEYLTTGSFMIRGK | 15 |
| SDCCAG1-475 | | 475 | LKGELIEMNLQIVDR | 15 |
| SDCCAG1-417 | | 417 | DLKALQQEKQALKKL | 15 |
| SDCCAG1-942 | | 942 | TSELISEEMEQLDGG | 15 |
| SDCCAG1-9 | | 9 | STIDLRAVLAELNAS | 15 |
| SDCCAG1-482 | | 482 | MNLQIVDRAIQVVRS | 15 |
| SDCCAG1-165 | | 165 | GNIVLTDYEYVILNI | 15 |
| SDCCAG1-71 | | 71 | KATLLLESGIRIHTT | 15 |
| SDCCAG1-627 | | 627 | NKPLLVDVDLSLSAY | 15 |
| SDCCAG1-21 | | 21 | NASLLGMRVNNVYDV | 15 |
| SDCCAG10 - serologically defined | NM_005869 | | | |
| colon cancer antigen 10, NY-CO-10 | | | | |
| SDCCAG10-311 | | 311 | KRELLAAKQKKVENA | 15 |
| SDCCAG10-400 | | 400 | FKSKLTQAIAETPEN | 15 |
| SDCCAG10-393 | | 393 | TLALLNQFKSKLTQA | 15 |
| SDCCAG10-159 | | 159 | EEEEVNRVSQSMKGK | 15 |
| SDCCAG3 - serologically defined colon | NM_006643 | | | |
| cancer antigen 3, NY-CO-3 | | | | |
| SDCCAG3-322 | | 322 | DYHDLESVVQQVEQN | 15 |
| SDCCAG3-350 | | 350 | HVVKLKQEISLLQA | 14 |
| SDCCAG3-192 | | 192 | PSWALSDTDSRVSP | 14 |
| SDCCAG3-418 | | 418 | LRVVMNSAQASIKQL | 15 |
| SDCCAG3-428 | | 428 | SIKQLVSGAETLNLV | 15 |
| SDCCAG3-262 | | 262 | ENSKLRRKLNEVQSF | 15 |
| SDCCAG3-255 | | 255 | SYDALKDENSKLRRK | 15 |
| SDCCAG3-411 | | 411 | ADVALQNLRVVMNSA | 15 |
| SDCCAG3-462 | | 462 | AEILKSIDRISEI | 13 |
| SDCCAG3-248 | | 248 | HLRTLQISYDALKDE | 15 |
| SDCCAG8 - serologically defined colon | NM_006642 | | | |
| cancer antigen 8, NY-CO-8 | | | | |
| SDCCAG8-419 | | 419 | ERDDLMSALVSVRSS | 15 |
| SDCCAG8-557 | | 557 | KMLILSQNIAQLEAQ | 15 |
| SDCCAG8-815 | | 815 | ECCTLAKKLEQISQK | 15 |
| SDCCAG8-423 | | 423 | LMSALVSVRSSLADT | 15 |
| SDCCAG8-945 | | 945 | ERQSLSEEVDRLRTQ | 15 |
| SDCCAG8-564 | | 564 | NIAQLEAQVEKVTKE | 15 |
| SDCCAG8-397 | | 397 | HEAVLSQTHTNVHMQ | 15 |
| SDCCAG8-582 | | 582 | AINQLEEIQSQLASR | 15 |
| SDCCAG8-798 | | 798 | QYLLLTSQNTFLTKL | 15 |
| SDCCAG8-776 | | 776 | LTQKIQQMEAQ | 13 |
| SDCCAG8-589 | | 589 | IQSQLASREMDV | 13 |
| SDCCAG8-156 | | 156 | NMPTMHDLVHTINDQ | 15 |
| SDCCAG8-561 | | 561 | LSQNIAQLEAQVEKV | 15 |
| SDCCAG8-184 | | 184 | CKEELSGMKNKIQVV | 15 |
| SDCCAG8-35 | | 35 | LTCALKEGDVTIG | 13 |
| SDCCAG8-28 | | 28 | ASRSIHQLTCALKEG | 15 |
| SDCCAG8-952 | | 952 | EVDRLRTQLPSMPQS | 15 |
| SDCCAG8-13 | | 13 | LEEILGQYQRSLREH | 15 |
| SDCCAG8-550 | | 550 | EREYMGSKMLILSQN | 15 |
| SEC14L1 - SEC14-like 1 | NM_003003 | | | |
| SEC14L1-488 | | 488 | GEEALLRYVLSVNEE | 15 |
| SEC14L1-560 | | 560 | GVKALLRIIEVVEAN | 15 |
| SEC14L1-190 | | 190 | EKIAMKQYTSNIKKG | 15 |
| SEC14L1-88 | | 88 | DAPRLLKKIAGVDYV | 15 |
| SEC14L1-730 | | 730 | ILIQIVDASSVITWD | 15 |
| SEC14L1-106 | | 106 | QKNSLNSRERTLHIE | 15 |
| SEC14L1-948 | | 948 | GFSQLSAATTSSSQS | 15 |
| SEC14L1-810 | | 810 | KVWQLGRDYSMVESP | 15 |
| SEC14L1-803 | | 803 | NNVQLIDKVWQLGRD | 15 |
| SEC14L1-882 | | 882 | SLPRVDDVLASLQVS | 15 |
| SEC14L1-579 | | 579 | LGRLLILRAPRVFPV | 15 |
| SEC14L1-1 | | 1 | MVQKYQSPVRVY | 12 |
| SEC14L1-493 | | 493 | LRYVLSVNEERLRRC | 15 |
| SEC14L1-263 | | 263 | SKKQAASMAVVIPEA | 15 |
| SEC14L1-898 | | 898 | HKCKVMYYTEVIGSE | 15 |
| SFRS2IP - splicing factor, | NM_004719 | | | |
| arginine/serine-rich 2, interacting protein | | | | |
| SFRS2IP-1417 | | 1417 | AAVKLAESKVSVAVE | 15 |
| SFRS2IP-339 | | 339 | PLSDLSENVESVVNE | 15 |
| SFRS2IP-491 | | 491 | LEKSLEEKNESLTEH | 15 |
| SFRS2IP-336 | | 336 | VSCPLSDLSENVESV | 15 |
| SFRS2IP-400 | | 400 | ESPKLESSEGEIIQT | 15 |
| SFRS2IP-1277 | | 1277 | LPLHLHTGVPLMQVA | 15 |
| SFRS2IP-1206 | | 1206 | LPINMMQPQMNVMQQ | 15 |
| SFRS2IP-1492 | | 1492 | YKEIVRKAVDKVCHS | 15 |
| SFRS2IP-1207 | | 1207 | PINMMQPQMNVMQQQ | 15 |
| SFRS2IP-158 | | 158 | DSSNICTVQTHVENQ | 15 |
| SFRS2IP-232 | | 232 | DLPVLVGEEGEVKKL | 15 |
| SFRS2IP-173 | | 173 | SANCLKSCNEQIEES | 15 |
| SLC2A11 - solute carrier family 2, | NM_030807 | | | |
| member 11, GLUT10; GLUT11 | | | | |
| SLC2A11-403 | | 403 | GNDSVYAYASSVFRK | 15 |
| SLC2A11-381 | | 381 | LRRQVTSLVVL | 12 |
| SLC2A11-147 | | 147 | KSLLVNNIFVVSAA | 14 |
| SLC2A11-110 | | 110 | LFGALLAGPLAITLG | 15 |
| SLC2A11-93 | | 93 | LVLLMWSLIVSLYPL | 15 |
| SLC2A11-501 | | 501 | FPWTLYLAMACIFAF | 15 |
| SLC2A11-174 | | 174 | EMIMLGRLLVGVNAG | 15 |
| SLC2A11-151 | | 151 | LVNNIFVVSAAILFG | 15 |
| SLC2A11-233 | | 233 | MSSAIFTALGIVMGQ | 15 |
| SLC2A11-229 | | 229 | GAVAMSSAIFTALGI | 15 |
| SLC2A11-91 | | 91 | DHLVLLMWSLIVSLY | 15 |
| SLC2A11-237 | | 237 | IFTALGIVMGQVVGL | 15 |
| SLC2A11-178 | | 178 | LGRLLVGVNAGVSMN | 15 |
| SLC2A11-567 | | 567 | VCGALMWIMLILVGL | 15 |
| SOX8 - SRY (sex determining region | NM_014587 | | | |
| Y)-box 8 | | | | |
| SOX8-173 | | 173 | HNAELSKTLGKLWRL | 15 |
| SOX8-349 | | 349 | SNVDISELSSEVMGT | 15 |
| SOX8-88 | | 88 | FPACIRDAVSQVLKG | 15 |
| SOX8-161 | | 161 | ARRKLADQYPHLHNA | 15 |
| SOX8-352 | | 352 | DISELSSEVMGT | 12 |
| SOX8-263 | | 263 | GGGAVYKAEAGLGDG | 15 |
| SOX8-17 | | 17 | SPSGTASSMSHVEDS | 15 |
| SOX8-177 | | 177 | LSKTLGKLWRLLSES | 15 |
| SOX8-96 | | 96 | VSQVLKGYDWSLVPM | 15 |
| SSRP1 - structure specific recognition | NM_003146 | | | |
| protein 1 | | | | |
| SSRP1-414 | | 414 | MSGSLYEMVSRVMKA | 15 |
| SSRP1-425 | | 425 | VMKALVNRKITVPGN | 15 |
| SSRP1-418 | | 418 | LYEMVSRVMKALVNR | 15 |
| SSRP1-786 | | 786 | SITDLSKKAGEIWKG | 15 |
| SSRP1-391 | | 391 | ISLTLNMNEEEVEKR | 15 |
| SSRP1-78 | | 78 | RRVALGHGLKLLTKN | 15 |
| SSRP1-410 | | 410 | LTKNMSGSLYEMVSR | 15 |
| SSRP1-84 | | 84 | HGLKLLTKNGHVYKY | 15 |
| SSTR5 - somatostatin receptor 5 | NM_001053 | | | |
| SSTR5-152 | | 152 | FGPVLCRLVMTLDGV | 15 |
| SSTR5-100 | | 100 | NIYILNLAVADVLYM | 15 |
| SSTR5-329 | | 329 | SERKVTRMVLVVVLV | 15 |
| SSTR5-352 | | 352 | FTVNIVNLAVAL | 15 |
| SSTR5-230 | | 230 | WVLSLCMSLPLLVFA | 15 |
| SSTR5-104 | | 104 | LNLAVADVLYMLGLP | 15 |
| SSTR5-332 | | 332 | KVTRMVLVVVLVFAG | 15 |
| SSTR5-176 | | 176 | TVMSVDRYLAVVHPL | 15 |
| SSTR5-75 | | 75 | CAAGLGGNTLVIYVV | 15 |
| STK16 - serine/threonine kinase 16, | NM_003691 | | | |
| MPSK; PKL12 | | | | |
| STK16-351 | | 351 | ALRQLLNSMMTVD | 13 |
| STK16-390 | | 390 | HIPLLLSQLEALQPP | 15 |
| STK16-348 | | 348 | HSSALRQLLNSMMTV | 15 |
| STK16-147 | | 147 | RGTLWNEIERLKDK | 14 |
| STK16-232 | | 232 | DLGSMNQACIHVEGS | 15 |
| STK16-304 | | 304 | WSLGCVLYAMMFG | 13 |
| STUB1 - STIP1 homology and U-Box | NM_005861 | | | |
| containing protein 1, NY-CO-7 | | | | |
| STUB1-223 | | 223 | LHSYLSRLIAA | 12 |
| STUB1-100 | | 100 | HEQALADCRRALELD | 15 |
| STUB1-93 | | 93 | CYLKMQQHEQALADC | 15 |
| STUB1-340 | | 340 | DRKDIEEHLQRVGHF | 15 |
| STUB1-273 | | 273 | YMADMDELFSQV | 12 |
| TAF10 - TAF10 | NM_006284 | | | |
| TAF10-164 | | 164 | FLMQLEDYTPTIPDA | 15 |
| TAF10-266 | | 266 | LTPALSEYGINVKKP | 15 |
| TAF10-157 | | 157 | SSTPLVDFLMQLEDY | 15 |
| TAF10-112 | | 112 | PEGAISNGVYVLPSA | 15 |
| TAF10-259 | | 259 | YTLTMEDLTPALSEY | 15 |
| TP53 - tumor protein p53 | NM_000546 | | | |
| TP53-171 | | 171 | YSPALNKMFCQLAKT | 15 |
| TP53-348 | | 348 | SGNLLGRNSFEVRVC | 15 |
| TP53-340 | | 340 | TIITLEDSSGNLLGR | 15 |
| TP53-224 | | 224 | AIYKQSQHMTEV | 12 |
| TP53-86 | | 86 | EAPRMPEAAPRVAPA | 15 |
| TP53-24 | | 24 | DLWKLLPENNVLSPL | 15 |
| TP53-31 | | 31 | ENNVLSPLPSQAMDD | 15 |
| TPS1 - tryptase, alpha | NM_003293 | | | |
| TPS1-1 | | 1 | MLSLLLLALPVL | 12 |
| TPS1-174 | | 174 | EPVNISSRVHTVMLP | 15 |
| TPS1-165 | | 165 | ADIALLELEEPVNIS | 15 |
| TPS1-11 | | 11 | ALPVLASRAYAAPAP | 15 |
| TPS1-103 | | 103 | DVKDLATLRVQLREQ | 15 |
| TPS1-237 | | 237 | PPFPLKQVKVPIMEN | 15 |
| TPSB1 - tryptase beta 1 | NM_003294 | | | |
| TPSB1-174 | | 174 | EPVNVSSHVHTVTLP | 15 |
| TPSB1-1 | | 1 | MLNLLLLALPVL | 12 |
| TPSB1-165 | | 165 | ADIALLELEEPVNVS | 15 |
| TPSB1-103 | | 103 | DVKDLAALRVQLREQ | 15 |
| TPSB1-11 | | 11 | ALPVLASRAYAAPAP | 15 |
| TPSB1-159 | | 159 | YTAQIGADIALLELE | 15 |
| TPSD1 - tryptase delta 1 | NM_012217 | | | |
| TPSD1-3 | | 3 | MLLLAPQMLSLLLL | 15 |
| TPSD1-181 | | 181 | EPVNISSHIHTVTLP | 15 |
| TPSD1-149 | | 149 | YQDQLLPVSRIIVHP | 15 |
| TPSD1-10 | | 10 | QMLSLLLLALPVLAS | 15 |
| TPSD1-172 | | 172 | ADIALLELEEPVNIS | 15 |
| UBE2I - ubiquitin-conjugating enzyme | NM_003345 | | | |
| E2I | | | | |
| UBE2I-150 | | 150 | PAITIKQILLGIQEL | 15 |
| UBE2I-154 | | 154 | IKQILLGIQELLNEP | 15 |
| UTP14A - UTP14, U3 small nucleolar | NM_006649 | | | |
| ribonucleoprot, homA, NY-CO-16 | | | | |
| UTP14A-66 | | 66 | KLLEAISSLDGK | 12 |
| UTP14A-5 | | 5 | TANRLAESLLALSQQ | 15 |
| UTP14A-107 | | 107 | EKLVLADLLEPVKTS | 15 |
| UTP14A-905 | | 905 | EKRNIHAAAHQV | 12 |
| UTP14A-668 | | 668 | EEPLLLQRPERV | 12 |
| UTP14A-144 | | 144 | VKKQLSRVKSK | 12 |
| UTP14A-818 | | 818 | IRDFLKEKREAVEAS | 15 |
| UTP14A-223 | | 223 | LEKEEPAIAPI | 12 |
| UTP14A-182 | | 182 | TAQVLSKWDPVVLKN | 15 |
| UTP14A-89 | | 89 | SEASLKVSEFNVSSE | 15 |
| UTP14A-627 | | 627 | VLSELRVLSQKLKEN | 15 |
| UTP14A-254 | | 254 | IFNLLHKNKQPVTDP | 15 |
| UTP14A-246 | | 246 | ARTPLEQEIFNLLHK | 15 |
| WFIKKN1 - WAP, follis/kazal, im, kunitz | NM_053284 | | | |
| and netrin domain cont. 1 | | | | |
| WFIKKN1-583 | | 583 | SDFAIVGRLTEVLEE | 15 |
| WFIKKN1-15 | | 15 | LLLRLTSGAGLLPGL | 15 |
| WFIKKN1-3 | | 3 | MPALRPLLPLLLLL | 14 |
| WFIKKN1-723 | | 723 | ILELLEKQACELLNR | 15 |
| WFIKKN1-640 | | 640 | GLKFLGTKYLEVTLS | 15 |
| WFIKKN1-576 | | 576 | LALSLCRSDFAIVGR | 15 |
| WFIKKN1-645 | | 645 | GTKYLEVTLSGMDWA | 15 |
| WFIKKN1-324 | | 324 | YGNVVVTSIGQLVLY | 15 |
| WFIKKN1-701 | | 701 | DGVAVLDAGSYVRAA | 15 |
| WFIKKN1-716 | | 716 | SEKRVKKILELLEKQ | 15 |
| WFIKKN1-506 | | 506 | YSPLLQQCHPFVYGG | 15 |
| ZNF28 - zinc finger protein 28 (KOX 24) | NM_006969 | | | |
| ZNF28-15 | | 15 | VYDKIFEYNSYLAKH | 15 |
| ZNF28-92 | | 92 | ECGIVFNQQSHLASH | 15 |
| ZNF292 - zinc finger protein 292 | XM_048070 | | | |
| ZNF292-2597 | | 2597 | QMMALNSCTTSINSD | 15 |
| ZNF292-562 | | 562 | PNGKLIEEISEVDCK | 15 |
| ZNF292-3236 | | 3236 | TPEEIESMTASVDVG | 15 |
| ZNF292-1500 | | 1500 | TTPLLQSSEVAVSIK | 15 |
| ZNF292-2768 | | 2768 | SQCVLINTSVTLTPT | 15 |
| ZNF292-2630 | | 2630 | IKTAMNSQILEVKSG | 15 |
| ZNF292-861 | | 861 | QCLALMGEEASIVSS | 15 |
| ZNF292-662 | | 662 | QLSLLTKTVYHIFFL | 15 |
| ZNF292-2165 | | 2165 | ASMILSTNAVNLQQP | 15 |
| ZNF292-1850 | | 1850 | FPAHLASVSTPLLSS | 15 |
| ZNF292-330 | | 330 | PLPLLEVYTVAIQSY | 15 |
| ZNF292-659 | | 659 | RCRQLSLLTKTVYHI | 15 |
| ZNF292-502 | | 502 | KTNQLSQATALAKLC | 15 |
| ZNF292-2529 | | 2529 | LVENLTQKLNNVNNQ | 15 |
| ZNF292-2160 | | 2160 | QPSLLASMILSTNAV | 15 |
| ZNF292-3885 | | 3885 | VLKQLQEMKPTVSLK | 15 |
| ZNF292-1902 | | 1902 | QGGMLCSQMENLPST | 15 |
| ZNF292-2479 | | 2479 | TTMGLIAKSVEIPTT | 15 |
| ZNF292-1105 | | 1105 | KKNSLYSTDFIVFND | 15 |
| ZNF292-347 | | 347 | ARPYLTSECENVALV | 15 |
| ZNF292-868 | | 868 | EEASIVSSIDELNDS | 15 |
| ZNF292-3630 | | 3630 | ITKLINEDSTSVETQ | 15 |
| ZNF292-1921 | | 1921 | QMEDLTKTVLPLNID | 15 |
| ZNF292-263 | | 263 | LGERLQELELQLRES | 15 |
| ZNF292-2553 | | 2553 | FKTSLESHTVLAPLT | 15 |
| ZNF292-3415 | | 3415 | KKNNLENKNAKIVQI | 15 |
| ZNF292-1612 | | 1612 | TPQNLERQVNNLMTF | 15 |
| ZNF292-1597 | | 1597 | QNSLVNSETLKIGDL | 15 |
| ZNF292-3193 | | 3193 | DCSRIFQAITGLIQH | 15 |
| ZNF292-3154 | | 3154 | HKSDLPAFSAEVEEE | 15 |
| ZNF292-2846 | | 2846 | TKDALFKHYGKIHQY | 15 |
| ZNF292-2533 | | 2533 | LTQKLNNVNNQLFMT | 15 |
| ZNF292-2163 | | 2163 | LLASMILSTNAVNLQ | 15 |
| ZNF292-862 | | 862 | CLALMGEEASIVSSI | 15 |
| AHSA2 - AHA1, activator of heat shock | NM_152392 | | | |
| 90 protein ATPase homolog 2 | | | | |
| AHSA2-18 | | 18 | VKRKLSGNTLQVQAS | 15 |
| AHSA2-7 | | 7 | PTKAMATQELTVKRK | 15 |
| AHSA2-33 | | 33 | SPVALGVRIPTVALH | 15 |
| AHSA2-115 | | 115 | FVPTLGQTELQL | 12 |
| CSNK1G1 - casein kinase 1, gamma 1 | NM_022048 | | | |
| CSNK1G1-189 | | 189 | IAIQLLSRMEYVHSK | 15 |
| CSNK1G1-183 | | 183 | LKTVLMIAIQLLSRM | 15 |
| CSNK1G1-342 | | 342 | KADTLKERYQKIGDT | 15 |
| CSNK1G1-273 | | 273 | EHKSLTGTARYM | 12 |
| CSNK1G1-390 | | 390 | FPEEMATYLRYVRRL | 15 |
| CSNK1G1-411 | | 411 | DYEYLRTLFTDLFEK | 15 |
| CSNK1G1-467 | | 467 | GSVHVDSGASAITRE | 15 |
| DKFZp451M2119 | NM_182585 | | | |
| DKFZp451M2119-80 | | 80 | APTQMSTVPSGLPLP | 15 |
| DKFZp451M2119-30 | | 30 | DEGLVEGKVVRLGQG | 15 |
| DKFZp451M2119-234 | | 234 | QILWLYSKSSLAL | 13 |
| DKFZP564M182 | NM_015659 | | | |
| DKFZP564M182-309 | | 309 | QIEHIIENIVAVTKG | 15 |
| DKFZP564M182-77 | | 77 | NYGLLLNENESLFLM | 15 |
| DKFZP564M182-86 | | 86 | ESLFLMVVLWKIPSK | 15 |
| DKFZP564M182-344 | | 344 | KSAALPIFSSFVSNW | 15 |
| DKFZP564M182-190 | | 190 | KLRLLSSFDFFLTDA | 15 |
| DKFZP564M182-585 | | 585 | KEEAVKEKSPSLGKK | 15 |
| DKFZP564M182-313 | | 313 | IIENIVAVTKGLSEK | 15 |
| DKFZP564M182-164 | | 164 | NKHGIKTVSQIISLQ | 15 |
| DKFZP564M182-260 | | 260 | INDCIGGTVLNISKS | 15 |
| MAGEA4 | NM_002362 | | | |
| MAGEA4-151 | | 151 | FREALSNKVDELAHF | 15 |
| MAGEA4-171 | | 171 | RAKELVTKAEMLERV | 15 |
| MAGEA4-391 | | 391 | SYVKVLEHVVRVNAR | 15 |
| MAGEA4-265 | | 265 | KTGLLIIVLGTIAME | 15 |
| MAGEA4-414 | | 414 | REAALLEEEEGV | 12 |
| MAGEA4-395 | | 395 | VLEHVVRVNARVRIA | 15 |
| MELK - maternal embryonic leucine | NM_014791 | | | |
| zipper kinase | | | | |
| MELK-783 | | 783 | NPDQLLNEIMSILPK | 15 |
| MELK-322 | | 322 | SSILLLQQMLQVDPK | 15 |
| MELK-157 | | 157 | VFRQIVSAVAYVHSQ | 15 |
| MELK-31 | | 31 | ACHILTGEMVAIKIM | 15 |
| MELK-784 | | 784 | PDQLLNEIMSILPKK | 15 |
| MELK-145 | | 145 | RLSEEETRVVFR | 12 |
| MELK-417 | | 417 | QYDHLTATYLLLLAK | 15 |
| MELK-722 | | 722 | LERGLDKVITVLTRS | 15 |
| MELK-234 | | 234 | CCGSLAYAAPELIQG | 15 |
| MELK-67 | | 67 | NTLGSDLPRIKTE | 13 |
| MELK-315 | | 315 | VPKWLSPSSILLLQQ | 15 |
| MELK-718 | | 718 | VFGSLERGLDKVITV | 15 |
| MELK-95 | | 95 | QLYHVLETANKIFMV | 15 |
| MELK-74 | | 74 | DLPRIKTEIEALKNL | 15 |
| MELK-642 | | 642 | RNQCLKETPIKIPVN | 15 |
| MELK-180 | | 180 | PENLLFDEYHKLKLI | 15 |
| MELK-241 | | 241 | AAPELIQGKSYLGSE | 15 |
| NEXN - nexilin (F actin binding protein) | NM_144573 | | | |
| NEXN-81 | | 81 | GDDSLLITVVPVKSY | 15 |
| NEXN-34 | | 34 | IQRELAKRAEQIED | 14 |
| NEXN-382 | | 382 | NLKSKFEKIGQL | 12 |
| NEXN-340 | | 340 | ETFGLSREYEELIKL | 15 |
| NEXN-261 | | 261 | SQEFLTPGKLEINFE | 15 |
| NEXN-661 | | 661 | KGSAASTCILTIESK | 15 |
| NFE2L2 - nuclear factor (erythroid- | NM_006164 | | | |
| derived 2)-like 2 | | | | |
| NFE2L2-409 | | 409 | SPATLSHSLSELLNG | 15 |
| NFE2L2-741 | | 741 | SLHLLKKQLSTLYLE | 15 |
| NFE2L2-745 | | 745 | LKKQLSTLYLEVFS | 14 |
| NFE2L2-164 | | 164 | CMQLLAQTFPFVDDN | 15 |
| NFE2L2-626 | | 626 | TRDELRAKALHIPFP | 15 |
| NFE2L2-506 | | 506 | EVEELDSAPGSVKQN | 15 |
| NFE2L2-249 | | 249 | DIEQVWEELLSIPEL | 15 |
| NFE2L2-315 | | 315 | FYSSIPSMEKEVGNC | 15 |
| NFRKB - nuclear factor related to kappa | NM_006165 | | | |
| B binding protein | | | | |
| NFRKB-413 | | 413 | GDLTLNDIMTRVNAG | 15 |
| NFRKB-559 | | 559 | LEILLLESQASLPML | 15 |
| NFRKB-1575 | | 1575 | SAVSLPSMNAAVSKT | 15 |
| NFRKB-1221 | | 1221 | TVTSLPATASPV | 12 |
| NFRKB-626 | | 626 | ALQYLAGESRAVPSS | 15 |
| NFRKB-1599 | | 1599 | TPISISTGAPTVRQV | 15 |
| NFRKB-553 | | 553 | SFFSLLLEILLLESQ | 15 |
| NFRKB-226 | | 226 | KQILASRSDLLEMA | 14 |
| NFRKB-1568 | | 1568 | GTVHTSAVSLPSM | 13 |
| NFRKB-1094 | | 1094 | TMLSPASSQTAPS | 13 |
| NFRKB-546 | | 546 | GINEISSSFFSLLLE | 15 |
| NFRKB-88 | | 88 | DVVSLSTWQEVLSDS | 15 |
| NFRKB-1675 | | 1675 | IKGNLGANLSGLGRN | 15 |
| NUP107 - nucleoporin 107 kDa | NM_020401 | | | |
| NUP107-413 | | 413 | KQRQLTSYVGSVRPL | 15 |
| NUP107-577 | | 577 | IYAALSGNLKQLLPV | 15 |
| NUP107-345 | | 345 | QRDSLVRQSQLVVDW | 15 |
| NUP107-471 | | 471 | DEVRLLKYLFTLIRA | 15 |
| NUP107-1218 | | 1218 | LLQKLRESSLMLLDQ | 15 |
| NUP107-632 | | 632 | VEQEIQTSVATLDET | 15 |
| NUP107-782 | | 782 | SIEVLKTYIQLLIRE | 15 |
| NUP107-225 | | 225 | SFLKHSSSTVFDL | 13 |
| NUP107-1099 | | 1099 | WKGHLDALTADVKEK | 15 |
| NUP107-734 | | 734 | LPGHLLRFMTHLILF | 15 |
| NUP107-339 | | 339 | VVEALFQRDSLVRQS | 15 |
| NUP107-250 | | 250 | QVNILSKIVSRATPG | 15 |
| NUP107-1110 | | 1110 | VKEKMYNVLLFVDGG | 15 |
| NUP107-1211 | | 1211 | SKEELRKLLQKLRES | 15 |
| NUP107-656 | | 656 | ANWTLEKVFEELQAT | 15 |
| NUP107-811 | | 811 | QDLAVAQYALFLESV | 15 |
| NUP107-472 | | 472 | EVRLLKYLFTLIRAG | 15 |
| NUP107-420 | | 420 | YVGSVRPLVTELDPD | 15 |
| NUP107-940 | | 940 | RAEALKQGNAIMRKF | 15 |
| RPA2 - replication protein A2, 32 kDa | NM_002946 | | | |
| RPA2-79 | | 79 | LSATLVDEVFRIGNV | 15 |
| RPA2-322 | | 322 | KHMSVSSIKQAVDFL | 15 |
| RPA2-267 | | 267 | PANGLTVAQNQVLNL | 15 |
| RPA2-71 | | 71 | VPCTISQLLSATLVD | 15 |
| RPA2-325 | | 325 | SVSSIKQAVDFLSNE | 15 |
| USP34 - ubiquitin specific protease 34 | NM_014709 | | | |
| USP34-3151 | | 3151 | FLLSLQAISTMVHFY | 15 |
| USP34-1119 | | 1119 | QKHALYSHSAEVQVR | 15 |
| USP34-1967 | | 1967 | QGTSLIQRLMSVAYT | 15 |
| USP34-2383 | | 2383 | ATCYLASTIQQLYMI | 15 |
| USP34-3318 | | 3318 | IVSMLFTSIAKLTPE | 15 |
| USP34-397 | | 397 | PLRHLLNLVSALEPS | 15 |
| USP34-4106 | | 4106 | FTETLVKLSVLVAYE | 15 |
| USP34-1351 | | 1351 | CMESLMIASSSLEQE | 15 |
| USP34-3874 | | 3874 | DLVELLSIFLSVLKS | 15 |
| USP34-3310 | | 3310 | YNNRLAEHIVSMLFT | 15 |
| USP34-2226 | | 2226 | GLTGLLRLATSVVKH | 15 |
| USP34-4264 | | 4264 | NRVEISKASASLNGD | 15 |
| USP34-4202 | | 4202 | MTHFLLKVQSQVFSE | 15 |
| USP34-1961 | | 1961 | LVQGTSLIQRL | 11 |
| USP34-4518 | | 4518 | PSTSISAVLSDLADL | 15 |
| USP34-414 | | 414 | TEQTLYLASMLIKAL | 15 |
| USP34-245 | | 245 | RLAGLSQITNQLHTF | 15 |
| USP34-4294 | | 4294 | LNPALIPTLQELLSK | 15 |
| USP34-2529 | | 2529 | FGGVITNNVVSLDCE | 15 |
| USP34-2517 | | 2517 | SPELKNTVKSLFGG | 14 |
| USP34-4219 | | 4219 | CANLISTLITNLISQ | 15 |
| USP34-3226 | | 3226 | KMIALVALLVEQ | 12 |
| USP34-3875 | | 3875 | LVELLSIFLSVLKST | 15 |
| USP34-3507 | | 3507 | LLGLLSRAKLYVDAA | 15 |
| USP34-4593 | | 4593 | LCRTIESTIHVVTRI | 15 |
| USP34-3106 | | 3106 | HSKHLTEYFAFLYEF | 15 |
| USP34-2227 | | 2227 | LTGLLRLATSVVKHK | 15 |
| USP34-2090 | | 2090 | NRSFLLLAASTL | 12 |
| USP34-1103 | | 1103 | FFDNLVYYIQTVREG | 15 |
| USP34-416 | | 416 | QTLYLASMLIKALWN | 15 |
| USP34-3801 | | 3801 | CWTTLISAFRILLES | 15 |
| USP34-2439 | | 2439 | TLLELQKMFTYLMES | 15 |
| USP34-465 | | 465 | SFASLLNTNIPIGNK | 15 |
| USP34-238 | | 238 | MSPTLTMRLAGLSQI | 15 |
| USP34-3556 | | 3556 | MTYCLISKTEKLMFS | 15 |
| USP34-3496 | | 3496 | TTVVLHQVYNVLLGL | 15 |
| USP34-3488 | | 3488 | RDLPLSPDTTVVLHQ | 15 |
| USP34-3327 | | 3327 | KMIALVALLVEQS | 13 |
| USP34-2925 | | 2925 | DPKAVSLMTAKLSTS | 15 |
| AARS - alanyl-tRNA synthetase | NM_001605 | | | |
| AARS-1289 | | 1289 | EALQLATSFAQLRLG | 15 |
| AARS-402 | | 402 | AYRVLADHARTITVA | 15 |
| AARS-1108 | | 1108 | QKDELRETLKSLKKV | 15 |
| AARS-327 | | 327 | TGMGLERLVSVLQNK | 15 |
| AARS-889 | | 889 | IANEMIEAAKAVYTQ | 15 |
| AARS-1046 | | 1046 | LKKCLSVMEAKVKAQ | 15 |
| AARS-539 | | 539 | LDRKIQSLGDS | 15 |
| AARS-1115 | | 1115 | TLKSLKKVMDDLDRA | 15 |
| AARS-1042 | | 1042 | KAESLKKCLSVMEAK | 15 |
| AARS-1017 | | 1017 | TEEAIAKGIRRIVAV | 15 |
| AARS-820 | | 820 | ATHILNFALRSVLGE | 15 |
| AARS-482 | | 482 | VVQSLGDAFPELKKD | 15 |
| AARS-658 | | 658 | YNYHLDSSGSYVFEN | 15 |
| AARS-1135 | | 1135 | QKRVLEKTKQFIDSN | 15 |
| ABL1 - v-abl Abelson murine leukemia | NM_005157 | | | |
| viral oncogene homolog 1 | | | | |
| ABL1-1515 | | 1515 | DFSKLLSSVKEISDI | 15 |
| ABL1-1342 | | 1342 | PLSTLPSASSALAGD | 15 |
| ABL1-349 | | 349 | KKYSLTVAVKTLKED | 15 |
| ABL1-465 | | 465 | NAVVLLYMATQISSA | 15 |
| ABL1-1427 | | 1427 | NSEQMASHSAVLEAG | 15 |
| ABL1-472 | | 472 | MATQISSAMEYLEKK | 15 |
| ABL1-937 | | 937 | SPHLWKKSSTLTSS | 14 |
| ABL1-1488 | | 1488 | KLENNLRELQIC | 12 |
| ABL1-1362 | | 1362 | AFIPLISTRVSLRKT | 15 |
| ABL1-260 | | 260 | TLAELVHHHSTVADG | 15 |
| ABL1-1409 | | 1409 | VVLDSTEALCLA | 12 |
| ABL1-557 | | 557 | APESLAYNKFSIKSD | 15 |
| ACAT2 - acetyl-Coenzyme A | NM_005891 | | | |
| acetyltransferase 2 ( | | | | |
| ACAT2-488 | | 488 | GCRILVTLLHTLERM | 15 |
| ACAT2-9 | | 9 | DPVVIVSAARTIIGS | 15 |
| ACAT2-424 | | 424 | DIFEINEAFAAVSAA | 15 |
| ACAT2-322 | | 322 | KPYFLTDGTGTVTPA | 15 |
| ACAT2-428 | | 428 | INEAFAAVSAAIVKE | 15 |
| ACAT2-491 | | 491 | ILVTLLHTLERMGRS | 15 |
| ACAT2-337 | | 337 | NASGINDGAAAVALM | 15 |
| AKAP13 - A kinase (PRKA) anchor | NM_006738 | | | |
| protein 13 | | | | |
| AKAP13-2954 | | 2954 | EQEDLAQSLSLVKDV | 15 |
| AKAP13-3489 | | 3489 | LTRSLSRPSSLIEQE | 15 |
| AKAP13-3096 | | 3096 | IFASLDQKSTVISLK | 15 |
| AKAP13-229 | | 229 | PRETLMHFAVRLGLL | 15 |
| AKAP13-3077 | | 3077 | QAVLLTDILVFLQEK | 15 |
| AKAP13-1520 | | 1520 | PNVLLSQEKNAVLGL | 15 |
| AKAP13-585 | | 585 | DQESLSSGDAVLQRD | 15 |
| AKAP13-3420 | | 3420 | LVFMLKRNSEQVVQS | 15 |
| AKAP13-3306 | | 3306 | PLMKSAINEVEIL | 13 |
| AKAP13-3069 | | 3069 | GRLKEVQAVLLTD | 13 |
| AKAP13-1688 | | 1688 | GADLIEEAASRIVDA | 15 |
| AKAP13-1052 | | 1052 | DQAVISDSTFSLANS | 15 |
| AKAP13-383 | | 383 | FKLMNIQQQLMKT | 13 |
| AKAP13-1024 | | 1024 | LDKPLTNMLEVVSHP | 15 |
| AKAP9 - A kinase (PRKA) anchor | NM_005751 | | | |
| protein (yotiao) 9 | | | | |
| AKAP9-5282 | | 5282 | DRALTDYITRLEAL | 14 |
| AKAP9-4202 | | 4202 | DRRSLLSEIQALHAQ | 15 |
| AKAP9-1964 | | 1964 | QEQLEEEVAKVIVS | 14 |
| AKAP9-3115 | | 3115 | EIDQLNEQVTKLQQ | 14 |
| AKAP9-1825 | | 1825 | QVQELESLISSLQQQ | 15 |
| AKAP9-3715 | | 3715 | NMTSLQKDLSQVRDH | 15 |
| AKAP9-2532 | | 2532 | LLEAISETSSQLEHA | 15 |
| AKAP9-4287 | | 4287 | LQEQLSSEKMVVAEL | 15 |
| AKAP9-2360 | | 2360 | ANNRLLKILLEVVKT | 15 |
| AMOTL2 - angiomotin like 2 | NM_016201 | | | |
| AMOTL2-415 | | 415 | GSAHLAQMEAVLREN | 15 |
| AMOTL2-583 | | 583 | EQEKLEREMALLRGA | 15 |
| AMOTL2-473 | | 473 | RIEKLESEIQRLSEA | 15 |
| AMOTL2-656 | | 656 | KVERLQQALGQLQAA | 15 |
| AMOTL2-480 | | 480 | EIQRLSEAHESLTRA | 15 |
| AMOTL2-330 | | 330 | EVRILQAQVPPVFLQ | 15 |
| ANKHD1 - ankyrin repeat and KH | NM_017747 | | | |
| domain containing 1 | | | | |
| ANKHD1-245 | | 245 | VSCALDEAAAALTRM | 15 |
| ANKHD1-2244 | | 2244 | TPNSLSTSYKTVSLP | 15 |
| ANKHD1-1352 | | 1352 | LTDTLDDLIAAVSTR | 15 |
| ANKHD1-234 | | 234 | DPEVLRRLTSSVSCA | 15 |
| ANKHD1-2955 | | 2955 | AAVQLSSAVNIMNGS | 15 |
| ANKHD1-1356 | | 1356 | LDDLIAAVSTRVPTG | 15 |
| ANKHD1-1061 | | 1061 | KLNELGQRISAIEK | 14 |
| ANKHD1-336 | | 336 | GYYELAQVLLAMHAN | 15 |
| ANKHD1-340 | | 340 | LAQVLLAMHANVEDR | 15 |
| ANKHD1-3006 | | 3006 | GPATLFNHFSSLFDS | 15 |
| ANKHD1-2308 | | 2308 | RSKKLSVPASVVSRI | 15 |
| ANKRD11 - ankyrin repeat domain 11 | NM_013275 | | | |
| ANKRD11-3272 | | 3272 | TREVIQQTLAAIVDA | 15 |
| ANKRD11-304 | | 304 | KQLLAAGAEVNTK | 13 |
| ANKRD11-3400 | | 3400 | PPPSLAEPLKELFRQ | 15 |
| ANKRD11-822 | | 822 | KSPFLSSAEGAVPKL | 15 |
| ANKRD11-2154 | | 2154 | FERMLSQKDLEIEER | 15 |
| ANKRD11-3407 | | 3407 | PLKELFRQQEAVRGK | 15 |
| ANKRD13 - ankyrin repeat domain 13 | NM_033121 | | | |
| ANKRD13-499 | | 499 | FPLSLVEQVIPIIDL | 15 |
| ANKRD13-720 | | 720 | IQESLLTSTEGLCPS | 15 |
| ANKRD13-781 | | 781 | WELRLQEEEAELQQV | 15 |
| ANKRD13-266 | | 266 | ERFDLSQEMERLTLD | 15 |
| ANKRD13-74 | | 74 | SLGHLESARVLLRHK | 15 |
| ANKRD13-404 | | 404 | DRNPLESLLGTVEHQ | 15 |
| ANKRD17 - ankyrin repeat domain 17 | NM_032217 | | | |
| ANKRD17-1379 | | 1379 | LNDTLDDIMAAV | 12 |
| ANKRD17-263 | | 263 | DPEVLRRLTSSVSCA | 15 |
| ANKRD17-3102 | | 3102 | PESMLSGKSSYLPNS | 15 |
| ANKRD17-386 | | 386 | GYYELAQVLLAMHAN | 15 |
| ANKRD17-1667 | | 1667 | MLAAMNGHTAAVKLL | 15 |
| ANKRD17-478 | | 478 | VVKVLLESGASIEDH | 15 |
| ANKRD17-390 | | 390 | LAQVLLAMHANVEDR | 15 |
| ANKRD17-188 | | 188 | ENPMLETASKLLLSG | 15 |
| ANKRD30A - ankyrin repeat domain | NM_052997 | | | |
| 30A | | | | |
| ANKRD30A-577 | | 577 | DSRSLFESSAKIQVC | 15 |
| ANKRD30A-158 | | 158 | NKASLTPLLLSITKR | 15 |
| ANKRD30A-1219 | | 1219 | DSTSLSKILDTVHS | 14 |
| ANKRD30A-1428 | | 1428 | ENCMLKKEIAMLKLE | 15 |
| ANKRD30A-115 | | 115 | VYSEILSVVAKL | 12 |
| ANKRD30A-1435 | | 1435 | EIAMLKLEIATLKHQ | 15 |
| ANKRD30A-230 | | 230 | IVGMLLQQNVDVFAA | 15 |
| APEX2 - APEX nuclease | NM_014481 | | | |
| APEX2-76 | | 76 | TRDALTEPLAIVEGY | 15 |
| APEX2-247 | | 247 | RAEALLAAGSHVIIL | 15 |
| APEX2-384 | | 384 | DYVLGDRTLVIDTF | 14 |
| APEX2-240 | | 240 | FYRLLQIRAEALLAA | 15 |
| ARID4B - AT rich interactive domain 4B, | NM_016374 | | | |
| BCAA; BRCAA1; SAP180 | | | | |
| ARID4B-1690 | | 1690 | HYLSLKSEVASIDRR | 15 |
| ARID4B-1676 | | 1676 | RITILQEKLQEIRKH | 15 |
| ARID4B-468 | | 468 | NLFKLFRLVHKLGGF | 15 |
| ARID4B-234 | | 234 | QIDELLGKVVCVDYI | 15 |
| ARNTL - aryl hydrocarbon receptor | NM_001178 | | | |
| nuclear translocator-like | | | | |
| ARNTL-665 | | 665 | IGRMIAEEIMEIHRI | 15 |
| ARNTL-808 | | 808 | DEAAMAVIMSLLEAD | 15 |
| ARNTL-579 | | 579 | EVEYIVSTNTVVLAN | 15 |
| ARNTL-153 | | 153 | KLDKLTVLRMAVQHM | 15 |
| ARNTL-814 | | 814 | VIMSLLEADAGLGGP | 15 |
| ARNTL-234 | | 234 | KILFVSESVFKILNY | 15 |
| ASPSCR1 - alveolar soft part sarcoma | NM_024083 | | | |
| chromosome region, candidate 1 | | | | |
| ASPSCR1-345 | | 345 | PTRPLTSSSAKLPKS | 15 |
| ASPSCR1-223 | | 223 | LTGGSATIRFV | 12 |
| ASPSCR1-648 | | 648 | LEHAISPSAADVLVA | 15 |
| ASPSCR1-158 | | 158 | TLWELLSHFPQIREC | 15 |
| ATF3 - activating transcription factor 3 | NM_001674 | | | |
| ATF3-78 | | 78 | LCHRMSSALESVTVS | 15 |
| ATF3-162 | | 162 | ESEKLESVNAELKAQ | 15 |
| ATF3-169 | | 169 | VNAELKAQIEELKNE | 15 |
| ATXN3 - ataxin 3 | NM_004993 | | | |
| ATXN3-32 | | 32 | SPVELSSIAHQLDEE | 15 |
| ATXN3-189 | | 189 | SDTYLALFLAQLQQE | 15 |
| ATXN3-469 | | 469 | LQAAVTMSLETVRND | 15 |
| ATXN3-254 | | 254 | RPKLIGEELAQLKEQ | 15 |
| ATXN3-99 | | 99 | FSIQVISNALKVWGL | 15 |
| B3GALT4 - UDP-Gal:betaGlcNAc beta | NM_003782 | | | |
| 1,3-galactosyltransferase | | | | |
| B3GALT4-352 | | 352 | TGYVLSASAVQL | 12 |
| B3GALT4-9 | | 9 | FRRLLLAALLLVIVW | 15 |
| B3GALT4-32 | | 32 | GEELLSLSLASLLPA | 15 |
| BAIAP3 - BAI1-associated protein 3 | NM_003933 | | | |
| BAIAP3-227 | | 227 | DEEALLSYLQQVFGT | 15 |
| BAIAP3-578 | | 578 | WRGELSTPAATILCL | 15 |
| BAIAP3-239 | | 239 | FGTSLEEHTEAIERV | 15 |
| BAIAP3-1261 | | 1261 | WELLLQAILQALGAN | 15 |
| BAIAP3-555 | | 555 | SHLLLLSHLLRLEHS | 15 |
| BAIAP3-1212 | | 1212 | LMKYLDEKLALLNAS | 15 |
| BAIAP3-406 | | 406 | DDVSLVEACRKLNEV | 15 |
| BCR - breakpoint cluster region | NM_004327 | | | |
| BCR-265 | | 265 | RISSLGSQAMQMERK | 15 |
| BCR-1196 | | 1196 | ELQMLTNSCVKLQTV | 15 |
| BCR-1111 | | 1111 | LKKKLSEQESLLLLM | 15 |
| BCR-1188 | | 1188 | RSFSLTSVELQMLTN | 15 |
| BCR-1059 | | 1059 | ELDALKIKISQIKSD | 15 |
| BDP1 - TFIIIB150; TFIIIB90 | NM_018429 | | | |
| BDP1-145 | | 145 | SLVKSSVSVPSE | 12 |
| BDP1-2842 | | 2842 | TRNTISKVTSNLRIR | 15 |
| BDP1-341 | | 341 | GSIILDEESLTVEVL | 15 |
| BDP1-2385 | | 2385 | KESALAKIDAELEEV | 15 |
| BDP1-1837 | | 1837 | DIQNISSEVLSMMHT | 15 |
| BDP1-2205 | | 2205 | EKKVLTVSNSQIETE | 15 |
| BDP1-2358 | | 2358 | QLLLKEKAELLTS | 13 |
| BRD2 - bromodomain containing 2, | NM_005104 | | | |
| NAT; RING3 | | | | |
| BRD2-711 | | 711 | RLAELQEQLRAVHEQ | 15 |
| BRD2-410 | | 410 | PPGSLEPKAARLPPM | 15 |
| BRD2-267 | | 267 | KLAALQGSVTSAHQV | 15 |
| BRD2-227 | | 227 | DIVLMAQTLEKIFLQ | 15 |
| BRD2-718 | | 718 | QLRAVHEQLAALSQG | 15 |
| BRD2-708 | | 708 | RAHRLAELQEQLRAV | 15 |
| BZW2 - basic leucine zipper and W2 | NM_014038 | | | |
| domains 2 | | | | |
| BZW2-426 | | 426 | ALKHLKQYAPLLAVF | 15 |
| BZW2-65 | | 65 | LEAVAKFLDST | 12 |
| CHTF18 - chromosome transmission | NM_022092 | | | |
| fidelity factor 18 homolog | | | | |
| CHTF18-328 | | 328 | EAQKLSDTLHSLRSG | 15 |
| CHTF18-306 | | 306 | LGVSLASLKKQVDGE | 15 |
| CHTF18-706 | | 706 | LPSRLVQRLQEVSLR | 15 |
| CHTF18-1061 | | 1061 | EKQQLASLVGTMLA | 15 |
| CHTF18-896 | | 896 | RDSSLGAVCVALDWL | 15 |
| CHTF18-321 | | 321 | RRERLLQEAQKLSDT | 15 |
| CHTF18-1045 | | 1045 | LAPKLRPVSTQLYST | 15 |
| CHTF18-1030 | | 1030 | PQALLLDALCLLLDI | 15 |
| CLIC6 - chloride intracellular channel 6 | NM_053277 | | | |
| CLIC6-408 | | 408 | GDGSLSPQAEAIEVA | 15 |
| CLIC6-787 | | 787 | HEKNLLKALRKLDNY | 15 |
| CTNNA1 - catenin (cadherin-associated | NM_001903 | | | |
| protein), alpha 1, 102 kDa | | | | |
| CTNNA1-172 | | 172 | AARALLSAVTRLLIL | 15 |
| CTNNA1-331 | | 331 | IYKQLQQAVTGISNA | 15 |
| CTNNA1-28 | | 28 | VERLLEPLVTQVTTL | 15 |
| CTNNA1-966 | | 966 | DIIVLAKQMCMIMME | 15 |
| CTNNA1-409 | | 409 | FRPSLEERLESIISG | 15 |
| CTNNA1-1119 | | 1119 | AKNLMNAVVQTVKAS | 15 |
| CTNNA1-1111 | | 1111 | SAMSLIQAAKNLMNA | 15 |
| CTTN - cortactin | NM_005231 | | | |
| CTTN-149 | | 149 | YQSKLSKHCSQVDSV | 15 |
| CTTN-468 | | 468 | PVEAVTSKTSNIRAN | 15 |
| CTTN-629 | | 629 | SQQGLAYATEAVYES | 15 |
| CTTN-706 | | 706 | DPDDIITNIEMIDDG | 15 |
| CTTN-660 | | 660 | YENDLGITAVALYDY | 15 |
| CTTN-427 | | 427 | KNASTFEDVTQVSSA | 15 |
| CTTNBP2 - cortactin binding protein 2 | NM_033427 | | | |
| CTTNBP2-1035 | | 1035 | CVRLLLSAEAQVNAA | 15 |
| CTTNBP2-2134 | | 2134 | NNPVLSATINNLRMP | 15 |
| CTTNBP2-254 | | 254 | EAQKLEDVMAKLEEE | 15 |
| CTTNBP2-1373 | | 1373 | VSQALTNHFQAISSD | 15 |
| CTTNBP2-1901 | | 1901 | GQQAVVKAALSILLN | 15 |
| CTTNBP2-1296 | | 1296 | DCKHLLENLNALKIP | 15 |
| DAD1 - defender against cell death 1 | NM_001344 | | | |
| DAD1-26 | | 26 | RLKLLDAYLLYILLT | 15 |
| DAD1-77 | | 77 | FNSFLSGFISGVGSF | 15 |
| DAD1-16 | | 16 | LEEYLSSTPQRLKLL | 15 |
| DDX5 - DEAD (Asp-Glu-Ala-Asp) box | NM_004396 | | | |
| polypeptide 5 | | | | |
| DDX5-241 | | 241 | PTRELAQQVQQVAAE | 15 |
| DDX5-190 | | 190 | TLSYLLPAIVHINHQ | 15 |
| DDX5-627 | | 627 | LISVLREANQAINPK | 15 |
| DDX5-322 | | 322 | GKTNLRRTTYLVLDE | 15 |
| DDX5-620 | | 620 | KQVSDLISVLREA | 13 |
| DDX5-634 | | 634 | ANQAINPKLLQLVED | 15 |
| DDX58 - DEAD (Asp-Glu-Ala-Asp) box | NM_014314 | | | |
| polypeptide 58 | | | | |
| DDX58-488 | | 488 | TIPSLSIFTLMIFDE | 15 |
| DDX58-965 | | 965 | NLVILYEYVGNVIKM | 15 |
| DDX58-1109 | | 1109 | KCKALACYTADVRVI | 15 |
| DDX58-1013 | | 1013 | LTSNAGVIEKE | 12 |
| DDX58-726 | | 726 | ICKALFLYTSHLRKY | 15 |
| DDX58-645 | | 645 | IIAQLMRDTESLAKR | 15 |
| DNAJA1 - DnaJ (Hsp40) homolog, | NM_001539 | | | |
| subfamily A, member 1 | | | | |
| DNAJA1-384 | | 384 | ISTLDNRTIVITSH | 14 |
| DNAJA1-231 | | 231 | IGPGMVQQIQSVCME | 15 |
| DNAJA1-152 | | 152 | VVHQLSVTLEDLYNG | 15 |
| DNAJA1-68 | | 68 | FKQISQAYEVLSDA | 14 |
| DNAJA1-21 | | 21 | TQEELKKAYRKLALK | 15 |
| DNAJA2 - DnaJ (Hsp40) homolog, | NM_005880 | | | |
| subfamily A, member 2 | | | | |
| DNAJA2-240 | | 240 | LAPGMVQQMQSVCSD | 15 |
| DNAJA2-335 | | 335 | IVLLLQEKEHEVFQR | 15 |
| DNAJA2-473 | | 473 | NPDKLSELEDLLPSR | 15 |
| DNAJA2-23 | | 23 | SENELKKAYRKLAKE | 15 |
| DNAJA2-489 | | 489 | EVPNIIGETEEVELQ | 15 |
| DNAJB1 - DnaJ (Hsp40) homolog, | NM_006145 | | | |
| subfamily B, member 1 | | | | |
| DNAJB1-349 | | 349 | LREALCGCTVNVPTL | 15 |
| DNAJB1-430 | | 430 | FPERIPQTSRTVL | 13 |
| DNAJB1-338 | | 338 | GSDVIYPARISLREA | 15 |
| DNAJB1-230 | | 230 | VTHDLRVSLEEIYSG | 15 |
| DNM1L - dynamin 1-like, DRP1; DVLP; | NM_005690 | | | |
| DYMPLE; HDYNIV; VPS | | | | |
| DNM1L-627 | | 627 | RFPKLHDAIVEVVTC | 15 |
| DNM1L-415 | | 415 | RINVLAAQYQSLLNS | 15 |
| DNM1L-389 | | 389 | GTKYLARTLNRLLMH | 15 |
| DNM1L-313 | | 313 | AMDVLMGRVIPVKLG | 15 |
| DNM1L-3 | | 3 | MEALIPVINKLQDV | 14 |
| DNM1L-10 | | 10 | VINKLQDVFNTVGAD | 15 |
| DRCTNNB1A - down-regulated by | NM_032581 | | | |
| Ctnnb1, a (DRCTNNB1A) | | | | |
| DRCTNNB1A-36 | | 36 | DKSSLVSSLYKV | 12 |
| DRCTNNB1A-588 | | 588 | SSHGLAKTAATVF | 13 |
| DRCTNNB1A-23 | | 23 | PETSLPNYATNLKDK | 15 |
| DRCTNNB1A-265 | | 265 | SLQSLCQICSRICVC | 15 |
| DRCTNNB1A-164 | | 164 | HTKVLSFTIPSLSKP | 15 |
| DUSP12 - dual specificity phosphatase | NM_007240 | | | |
| 12 | | | | |
| DUSP12-311 | | 311 | CRRSLFRSSSILDHR | 15 |
| DUSP12-259 | | 259 | ELQNLPQELFAVDPT | 15 |
| DUSP12-160 | | 160 | CHAGVSRSVAIITAF | 15 |
| DUSP12-114 | | 114 | LLSHLDRCVAFIG | 13 |
| ELKS - Rab6-interacting protein 2 | NM_015064 | | | |
| (ELKS) | | | | |
| ELKS-241 | | 241 | KESKLSSSMNSIKTF | 15 |
| ELKS-1120 | | 1120 | MKAKLSSTQQSLAEK | 15 |
| ELKS-778 | | 778 | SSLKERVKSLQAD | 13 |
| ELKS-984 | | 984 | EVDRLLEILKEV | 12 |
| ELKS-624 | | 624 | ELLALQTKLETLTNQ | 15 |
| ELKS-1102 | | 1102 | QVEELLMAMEKVKQE | 15 |
| ELKS-1113 | | 1113 | VKQELESMKAKLSST | 15 |
| ELKS-803 | | 803 | LEEALAEKERTIERL | 15 |
| EXOSC6 - exosome component 6 | NM_058219 | | | |
| EXOSC6-224 | | 224 | ALTAAALALADA | 12 |
| EXOSC6-273 | | 273 | AAAGLTVALMPV | 12 |
| EXOSC6-185 | | 185 | PRAQLEVSALLLEDG | 15 |
| EXOSC6-302 | | 302 | LNQVAGLLGSG | 12 |
| EXOSC6-338 | | 338 | LYPVLQQSLVRAARR | 15 |
| EXOSC6-231 | | 231 | AALALADAGVEMYDL | 15 |
| EXOSC6-229 | | 229 | TAAALALADAGVEMY | 15 |
| EXOSC10 - exosome component 10 | NM_001001998 | | | |
| EXOSC10-883 | | 883 | TTCLIATAVITLFNE | 15 |
| EXOSC10-100 | | 100 | QGDRLLQCMSRVMQY | 15 |
| EXOSC10-168 | | 168 | RVGILLDEASGVNKN | 15 |
| EXOSC10-876 | | 876 | KEDNLLGTTCLIATA | 15 |
| EXOSC10-725 | | 725 | PNHMMLKIAEELPKE | 15 |
| FAHD1 - fumarylacetoacetate hydrolase | NM_031208 | | | |
| domain containing 1 | | | | |
| FAHD1-234 | | 234 | SIPYIISYVSKIITL | 15 |
| FAHD1-228 | | 228 | TSSMIFSIPYIISYV | 15 |
| FAHD1-251 | | 251 | GDIILTGTPKGVGPV | 15 |
| FRS2 - fibroblast growth factor receptor | NM_006654 | | | |
| substrate 2 | | | | |
| FRS2-32 | | 32 | DGNELGSGIMELTDT | 15 |
| FRS2-649 | | 649 | RTAAMSNLQKALPRD | 15 |
| FRS2-497 | | 497 | EDDNLGPKTPSLNGY | 15 |
| FRS2-146 | | 146 | EIMQNNSINVVEE | 13 |
| FRS2-504 | | 504 | KTPSLNGYHNNLDPM | 15 |
| FRS2-539 | | 539 | VNTENVTVPAS | 12 |
| GLIPR1 - GLI pathogenesis-related 1 | NM_006851 | | | |
| (glioma) | | | | |
| GLIPR1-329 | | 329 | SVILILSVIITILVQ | 15 |
| GLIPR1-330 | | 330 | VILILSVIITILVQL | 15 |
| GLIPR1-319 | | 319 | RYTSLFLIVNSVILI | 15 |
| GLIPR1-4 | | 4 | MRVTLATIAWMVSFV | 15 |
| GLIPR1-227 | | 227 | GFDALSNGAHFICNY | 15 |
| GMRP-1 - K+ channel tetramerization | NM_032320 | | | |
| protein | | | | |
| GMRP-1-574 | | 574 | SITNLAAAAADIPQD | 15 |
| GMRP-1-393 | | 393 | FEFYLEEMILPLMVA | 15 |
| GMRP-1-352 | | 352 | KCRDLSALMHEL | 12 |
| GMRP-1-467 | | 467 | YSTKLYRFFKYIENR | 15 |
| GMRP-1-571 | | 571 | KSKSITNLAAAAADI | 15 |
| GNPTAG - N-acetylglucosamine-1- | NM_032520 | | | |
| phosphotransferase, gamma subunit | | | | |
| GNPTAG-335 | | 335 | AHKELSKEIKRLKGL | 15 |
| GNPTAG-4 | | 4 | MAAGLARLLLLLGLS | 15 |
| GNPTAG-87 | | 87 | HLFRLSGKCFSLVES | 15 |
| GOLGA1 - golgi autoantigen, golgin | NM_002077 | | | |
| subfamily a, 1 | | | | |
| GOLGA1-561 | | 561 | RTQALEAQIVALERT | 15 |
| GOLGA1-400 | | 400 | VITHLQEKVASLEKR | 15 |
| GOLGA1-967 | | 967 | EAFHLIKAVSVLLNF | 15 |
| GOLGA1-94 | | 94 | LEARLSDYAEQVRNL | 15 |
| GOLGA1-649 | | 649 | VSVAMAQALEEVRKQ | 15 |
| GOLGA1-351 | | 351 | KEQELQALIQQLS | 13 |
| GOLGA1-743 | | 743 | ALRTLKAEEAAVVAE | 15 |
| GOLGA1-733 | | 733 | QIHQLQAELEALRTL | 15 |
| GOLGA1-785 | | 785 | LRGPLQAEALSVNES | 15 |
| GOLGA1-904 | | 904 | PGPEMANMAPSVT | 13 |
| GOLGA2 - golgi autoantigen, golgin | NM_004486 | | | |
| subfamily a, 2 | | | | |
| GOLGA2-339 | | 339 | RVGELERALSAVSTQ | 15 |
| GOLGA2-1130 | | 1130 | EYIALYQSQRAVLKE | 15 |
| GOLGA2-492 | | 492 | LEAHLGQVMESVRQL | 15 |
| GOLGA2-1187 | | 1187 | KLLELQELVLRLVGD | 15 |
| GOLGA2-1061 | | 1061 | THRALQGAMEKLQS | 14 |
| GOLGA2-569 | | 569 | RVQELETSLAELRNQ | 15 |
| GOLGA2-788 | | 788 | LQEKLSELKETVELK | 15 |
| GOLGA2-721 | | 721 | QNRELKEQLAELQSG | 15 |
| GOLGA2-156 | | 156 | STESLRQLSQQLNGL | 15 |
| GOLGA4 - golgi autoantigen, golgin | NM_002078 | | | |
| subfamily a, 4 | | | | |
| GOLGA4-940 | | 940 | ELESLSSELSEVLKA | 15 |
| GOLGA4-1131 | | 1131 | ERILLTKQVAEVEAQ | 15 |
| GOLGA4-2867 | | 2867 | LQTQLAQKTTLISDS | 15 |
| GOLGA4-622 | | 622 | ERISLQQELSRVKQE | 15 |
| GOLGA4-2991 | | 2991 | TKTMAKVITTVLKF | 14 |
| GOLGA4-1892 | | 1892 | NSISLSEKEAAISSL | 15 |
| GOLGA4-307 | | 307 | YISVLQTQVSLLKQR | 15 |
| GOLGA4-2065 | | 2065 | LETELKSQTARIMEL | 15 |
| GOLGA4-1830 | | 1830 | LKKELSENINAVTLM | 15 |
| GOLGA4-1572 | | 1572 | ENTFLQEQLVELKML | 15 |
| GOLGA4-2299 | | 2299 | EVHILEEKLKSVESS | 15 |
| GOLGA4-954 | | 954 | ARHKLEEELSVLKDQ | 15 |
| GOLGA4-937 | | 937 | QTELESLSSELSEV | 14 |
| GOLGB1 - golgi autoantigen, golgin | NM_004487 | | | |
| subfamily b, macrogolgin | | | | |
| GOLGB1-3907 | | 3907 | EVQSLKKAMSSL | 12 |
| GOLGB1-3322 | | 3322 | KTNQLMETLKTIKKE | 15 |
| GOLGB1-3558 | | 3558 | SISQLTRQVTALQEE | 15 |
| GOLGB1-2956 | | 2956 | LQENLDSTVTQLAAF | 15 |
| GOLGB1-2618 | | 2618 | LEERLMNQLAELNGS | 15 |
| GOLGB1-2131 | | 2131 | ENQSLSSSCESLKLA | 15 |
| GOLGB1-640 | | 640 | NIASLQKRVVELENE | 15 |
| GOLGB1-2065 | | 2065 | LTKSLADVESQVSAQ | 15 |
| GOLGB1-1925 | | 1925 | KEAALTKIQTEIIEQ | 15 |
| GOLGB1-1021 | | 1021 | ERDQLLSQVKELSMV | 15 |
| GOLGB1-2381 | | 2381 | EKDSLSEEVQDLKHQ | 15 |
| GOLGB1-3551 | | 3551 | EIESLKVSISQLTRQ | 15 |
| GOLGB1-2772 | | 2772 | KISALERTVKALEFV | 15 |
| GRASP - GRP1-associated scaffold | NM_181711 | | | |
| protein | | | | |
| GRASP-319 | | 319 | KDPSIYDTLESVRSC | 15 |
| GRASP-502 | | 502 | FRRRLLKFIPGLNRS | 15 |
| GRASP-259 | | 259 | RKAELEARLQYLKQT | 15 |
| GRASP-323 | | 323 | IYDTLESVRSCLYGA | 15 |
| GRIM19 - cell death-regulatory protein | NM_015965 | | | |
| GRIM19 (GRIM19) | | | | |
| GRIM19-76 | | 76 | VPRTISSASATLIMA | 15 |
| GRIM19-20 | | 20 | KTPQLQPGSAFLPRV | 15 |
| GRIM19-236 | | 236 | LRENLEEEAIIMKDV | 15 |
| GRIM19-160 | | 160 | GYSMLAIGIGTLIYG | 15 |
| GSPT1 - G1 to S phase transition 1 | NM_002094 | | | |
| GSPT1-267 | | 267 | REHAMLAKTAGVKHL | 15 |
| GSPT1-324 | | 324 | CKEKLVPFLKKVGFN | 15 |
| GSPT1-655 | | 655 | KTIAIGKVLKLVPEK | 15 |
| HAGH - hydroxyacylglutathione | NM_005326 | | | |
| hydrolase | | | | |
| HAGH-105 | | 105 | RIGALTHKITHLSTL | 15 |
| HAGH-8 | | 8 | VLPALTDNYMYLVID | 15 |
| HAGH-115 | | 115 | HLSTLQVGSLNV | 12 |
| HNRPAB - heterogeneous nuclear | NM_004499 | | | |
| ribonucleoprotein A/B | | | | |
| HNRPAB-156 | | 156 | FGFILFKDAASVEKV | 15 |
| HNRPAB-273 | | 273 | VKKVLEKKFHTV | 12 |
| HNRPAB-167 | | 167 | VEKVLDQKEHRLDGR | 15 |
| HNRPAB-252 | | 252 | MDPKLNKRRGFVFIT | 15 |
| HSPCA - heat shock 90 kDa protein 1, | NM_005348 | | | |
| alpha | | | | |
| HSPCA-184 | | 184 | YSAYLVAEKVTVITK | 15 |
| HSPCA-25 | | 25 | FQAEIAQLMSLIINT | 15 |
| HSPCA-788 | | 788 | MKDILEKKVEKVVVS | 15 |
| HSPCA-901 | | 901 | YETALLSSGFSLEDP | 15 |
| HSPCA-895 | | 895 | DLVILLYETALLSSG | 15 |
| HSPD1 - heat shock 60 kDa protein 1 | NM_002156 | | | |
| HSPD1-726 | | 726 | GVASLLTTAEVVVTE | 15 |
| HSPD1-543 | | 543 | RLAKLSDGVAVLKVG | 15 |
| HSPD1-571 | | 571 | VTDALNATRAAVEEG | 15 |
| HSPD1-661 | | 661 | IVEKIMQSSSEVGYD | 15 |
| HSPD1-337 | | 337 | KISSIQSIVPALEIA | 15 |
| HSPD1-248 | | 248 | IGNIISDAMKKVGRK | 15 |
| HUMAUANTIG - nucleolar GTPase | NM_013285 | | | |
| HUMAUANTIG-641 | | 641 | APQLLPSSSLEVVPE | 15 |
| HUMAUANTIG-478 | | 478 | QYITLMRRIFLIDCP | 15 |
| HUMAUANTIG-710 | | 710 | ANTEMQQILTRVRQN | 15 |
| HUMAUANTIG-502 | | 502 | ETDIVLKGVVQVEKI | 15 |
| IFI16 - interferon, gamma-inducible | NM_005531 | | | |
| protein 16 | | | | |
| IFI16-95 | | 95 | DIPTLEDLAETLKKE | 15 |
| IFI16-9 | | 9 | KNIVLLKGLEVINDY | 15 |
| IFI16-715 | | 715 | EVMVLNATESFVYEP | 15 |
| IFI16-500 | | 500 | KKNQMSKLISEMHSF | 15 |
| IKBKAP - inhibitor of kappa light | NM_003640 | | | |
| polypeptide gene enhancer | | | | |
| IKBKAP-1658 | | 1658 | EDLALLEALSEVVQN | 15 |
| IKBKAP-1584 | | 1584 | QESDLFSETSSVVSG | 15 |
| IKBKAP-313 | | 313 | REFALQSTSEPVAGL | 15 |
| IKBKAP-719 | | 719 | VIHHLTAASSEMDEE | 15 |
| IKBKAP-1116 | | 1116 | VCDAMRAVMESINPH | 15 |
| ILF3 - interleukin enhancer binding | NM_004516 | | | |
| factor 3, 90 kDa | | | | |
| ILF3-246 | | 246 | MEKVLAGETLSVNDP | 15 |
| ILF3-173 | | 173 | VADNLAIQLAAVTED | 15 |
| ILF3-622 | | 622 | KTAKLHVAVKVLQDM | 15 |
| ILF3-566 | | 566 | LQYKLVSQTGPVHAP | 15 |
| IQWD1 - IQ motif and WD repeats 1 | NM_018442 | | | |
| IQWD1-667 | | 667 | PASFMLRMLASLN | 13 |
| IQWD1-67 | | 67 | LEVSETAMEVDTP | 13 |
| IQWD1-653 | | 653 | NELMLEETRNTITVP | 15 |
| IQWD1-237 | | 237 | EWSSIASSSRGIGSH | 15 |
| IQWD1-575 | | 575 | EHLMLLEADNHVVNC | 15 |
| KLHL2 - kelch-like 2, | NM_007246 | | | |
| KLHL2-661 | | 661 | GVGVLNNLLYAVGGH | 15 |
| KLHL2-544 | | 544 | GAAVLNGLLYAVGGF | 15 |
| KLHL2-409 | | 409 | TPMNLPKLMVVVGGQ | 15 |
| KLHL2-252 | | 252 | ADVVLSEEFLNLGIE | 15 |
| LIMS1 - LIM and senescent cell antigen- | NM_004987 | | | |
| like domains 1 | | | | |
| LIMS1-419 | | 419 | LKKRLKKLAETLGRK | 15 |
| LIMS1-230 | | 230 | CGKELTADARELKGE | 15 |
| LIMS1-182 | | 182 | KCHAIIDEQPLIFKN | 15 |
| LMNA - lamin A/C | NM_005572 | | | |
| LMNA-406 | | 406 | RIDSLSAQLSQLQKQ | 15 |
| LMNA-731 | | 731 | AMRKLVRSVTVVEDD | 15 |
| LMNA-324 | | 324 | FESRLADALQELRAQ | 15 |
| LMNA-182 | | 182 | LEALLNSKEAALSTA | 15 |
| LMNA-410 | | 410 | LSAQLSQLQKQLAAK | 15 |
| LMNA-417 | | 417 | LQKQLAAKEAKLRDL | 15 |
| LMNA-403 | | 403 | SRIRIDSLSAQLSQL | 15 |
| LMNA-238 | | 238 | LEAALGEAKKQLQDE | 15 |
| LMNA-487 | | 487 | EYQELLDIKLALDME | 15 |
| MED6 - mediator of RNA polymerase II | NM_005466 | | | |
| transcription, subunit 6 | | | | |
| MED6-77 | | 77 | QRLTLEHLNQMVGIE | 15 |
| MED6-91 | | 91 | EYILLHAQEPILFII | 15 |
| MED6-160 | | 160 | INSRVLTAVHGIQSA | 15 |
| MED6239 | | 239 | QRQRVDALLLDLRQK | 15 |
| MKRN1 - makorin, ring finger protein, 1 | NM_013446 | | | |
| MKRN1-175 | | 175 | ASSSLSSIVGPLVEM | 15 |
| MKRN1-101 | | 101 | YSHDLSDSPYSVVCK | 15 |
| MKRN1-163 | | 163 | TATELTTKSSLAASS | 15 |
| MKRN1-483 | | 483 | KQKLILKYKEAMSNK | 15 |
| NAP1L3 - nucleosome assembly protein | NM_004538 | | | |
| 1-like 3 | | | | |
| NAP1L3-145 | | 145 | AVRNRVQALRNI | 12 |
| NAP1L3-648 | | 648 | ILKSIYYYTGEVNGT | 15 |
| NAP1L3-173 | | 173 | AIHDLERKYAELNKP | 15 |
| NEDD9 - neural precursor cell | NM_006403 | | | |
| expressed, dev. down-regulated 9 | | | | |
| NEDD9-1100 | | 1100 | STTALQEMVHQVTDL | 15 |
| NEDD9-973 | | 973 | HFISLLNAIDALFSC | 15 |
| NEDD9-566 | | 566 | LQQALEMGVSSLMAL | 15 |
| NEDD9-1055 | | 1055 | SSNQLCEQLKTIVMA | 15 |
| NEDD9-980 | | 980 | AIDALFSCVSSAQPP | 15 |
| NEDD9-626 | | 626 | VELFLKEYLHFVKGA | 15 |
| NS - nucleostemin | NM_014366 | | | |
| NS-392 | | 392 | VSMGLTRSMQVVPLD | 15 |
| NS-257 | | 257 | WLNYLKKELPTVVFR | 15 |
| NS-401 | | 401 | QVVPLDKQITIIDSP | 15 |
| NS-250 | | 250 | PKENLESWLNYLKKE | 15 |
| NUBP2 - nucleotide binding protein 2 | NM_012225 | | | |
| NUBP2-338 | | 338 | AFAALTSIAQKILDA | 15 |
| NUBP2-109 | | 109 | QSISLMSVGFLLEKP | 15 |
| NUBP2-155 | | 155 | KNALIKQFVSDVAWG | 15 |
| OGFR - opioid growth factor receptor | NM_007346 | | | |
| OGFR-570 | | 570 | SQGSLRTGTQEVGGQ | 15 |
| OGFR-337 | | 337 | RQSALDYFMFAVRCR | 15 |
| OGFR-565 | | 565 | EGCALSQGSLRTGTQ | 15 |
| PARC - p53-associated parkin-like | NM_015089 | | | |
| cytoplasmic protein | | | | |
| PARC-956 | | 956 | GLSALSQAVEEVTER | 15 |
| PARC-722 | | 722 | GEKALGEISVSVEMA | 15 |
| PARC-981 | | 981 | LREKLVKMLVELLTN | 15 |
| PARC-1368 | | 1368 | NKTLLLSVLRVITRL | 15 |
| PARC-1140 | | 1140 | SESLLLTVPAAVIL | 14 |
| PARC-3152 | | 3152 | FAVNLRNRVSAIHEV | 15 |
| PARC-2454 | | 2454 | SPELLLQALVPLTSG | 15 |
| PARC-1654 | | 1654 | HRGVLVRQLTLLVAS | 15 |
| PARC-731 | | 731 | VSVEMAESLLQVLSS | 15 |
| PIAS1 - protein inhibitor of activated | NM_016166 | | | |
| STAT, 1 | | | | |
| PIAS1-338 | | 338 | NITSLVRLSTTVPNT | 15 |
| PIAS1-6 | | 6 | DSAELKQMVMSLRVS | 15 |
| PIAS1-166 | | 166 | ELPHLTSALHPVHPD | 15 |
| PIAS1-428 | | 428 | PDSEIATTSLRVSLL | 15 |
| PPIL4 - peptidylprolyl isomerase | NM_139126 | | | |
| (cyclophilin)-like 4 | | | | |
| PPIL4-8 | | 8 | LETTLGDVVIDLYTE | 15 |
| PPIL4-306 | | 306 | TQAILLEMVGDLPDA | 15 |
| PPIL4-419 | | 419 | IHVDFSQSVAKVKWK | 15 |
| PPIL4-150 | | 150 | GSQFLITTGENLDYL | 15 |
| PSME3 - proteasome (prosome, | NM_005789 | | | |
| macropain) activator subunit 3 | | | | |
| PSME3-156 | | 156 | SNQQLVDIIEKVKPE | 15 |
| PSME3-150 | | 150 | PNGMLKSNQQLVDII | 15 |
| PSME3-3 | | 3 | MASLLKVDQEVKLK | 14 |
| PSME3-318 | | 318 | LHDMILKNIEKIKRP | 15 |
| RAB40C - member RAS oncogene | NM_021168 | | | |
| family | | | | |
| RAB40C-310 | | 310 | KSFSMANGMNAVMMH | 15 |
| RAB40C-319 | | 319 | NAVMMHGRSYSLASG | 15 |
| RAB40C-225 | | 225 | FNVIESFTELSRI | 13 |
| RABEP1 - rabaptin, RAB GTPase | NM_004703 | | | |
| binding effector protein 1 | | | | |
| RABEP1-13 | | 13 | PDVSLQQRVAELEKI | 15 |
| RABEP1-810 | | 810 | SALVLRAQASEILLE | 15 |
| RABEP1-1044 | | 1044 | QLESLQEIKISLEEQ | 15 |
| RABEP1-1016 | | 1016 | ISSLKAELERIKVE | 14 |
| RABEP1-861 | | 861 | QMAVLMQSREQVSEE | 15 |
| RABEP1-657 | | 657 | TASLLSSVTQGMESA | 15 |
| RABEP1-1034 | | 1034 | LESTLREKSQQLESL | 15 |
| RABEP1-246 | | 246 | DAEKLRSVVMPMEKE | 15 |
| RBM25 - RNA binding motif protein 25 | XM_027330 | | | |
| RBM25-34 | | 34 | VPMSIMAPAPTVLV | 14 |
| RBM25-978 | | 978 | KRKHIKSLIEKIPTA | 15 |
| RBM25-266 | | 266 | IEVLIREYSSELNAP | 15 |
| RBM25-258 | | 258 | RDQMIKGAIEVLIRE | 15 |
| RBPSUH - recombining binding protein | NM_005349 | | | |
| suppressor of hairless | | | | |
| RBPSUH-658 | | 658 | NSTSVTSSTATVVS | 14 |
| RBPSUH-628 | | 628 | AGAILRANSSQVPPN | 15 |
| RBPSUH-255 | | 255 | LFNRLRSQTVSTRYL | 15 |
| RBPSUH-659 | | 659 | STSVTSSTATVVS | 13 |
| RBPSUH-350 | | 350 | IIRKVDKQTALLDA | 14 |
| RBPSUH-236 | | 236 | KKQSLKNADLCIASG | 15 |
| SDCCAG1 - serologically defined colon | NM_004713 | | | |
| cancer antigen 1, NY-CO-1 | | | | |
| SDCCAG1-13 | | 13 | LRAVLAELNASLLGM | 15 |
| SDCCAG1-934 | | 934 | LASCTSELISE | 12 |
| SDCCAG1-232 | | 232 | TLERLTEIVASAPKG | 15 |
| SDCCAG1-860 | | 860 | TGEYLTTGSFMIRGK | 15 |
| SDCCAG1-475 | | 475 | LKGELIEMNLQIVDR | 15 |
| SDCCAG1-229 | | 229 | PLLTLERLTEIVASA | 15 |
| SR-A1 - serine arginine-rich pre-mRNA | NM_021228 | | | |
| splicing factor | | | | |
| SR-A1-1126 | | 1126 | RKVKLQSKVAVLIRE | 15 |
| SR-A1-394 | | 394 | EEEGLSQSISRISET | 15 |
| SR-A1-1525 | | 1525 | KAQELIQATNQILSH | 15 |
| SR-A1-1683 | | 1683 | YKDILRKAVHKICHS | 15 |
| SR-A1-1504 | | 1504 | GVLALTALLFKMEEA | 15 |
| HUB - Hu antigen B (ELAVL2) | NM_004432 | | | |
| HUB-146 | | 146 | LRLQTKTIKVSYA | 13 |
| HUB-467 | | 467 | NGYRLGDRVLQVSFK | 15 |
| HUB-78 | | 78 | ELKSLFGSIGEIESC | 15 |
| HUB-325 | | 325 | RLDNLLNMAYGVKRF | 15 |
| HUB-185 | | 185 | ELEQLFSQYGRIITS | 15 |
| HUB-75 | | 75 | TQEELKSLFGSIGEI | 15 |
| HUC - Hu antigen C (ELAVL3) | NM_001420 | | | |
| HUC-146 | | 146 | LKLQTKTIKVSYA | 13 |
| HUC-475 | | 475 | NGYRLGERVLQVSFK | 15 |
| HUC-5 | | 5 | VTQILGAMESQVGGG | 15 |
| HUC-338 | | 338 | SPLSLIARFSPIAID | 15 |
| HUC-325 | | 325 | RLDNLLNMAYGVKSP | 15 |
| HUC-78 | | 78 | EFKSLFGSIGDIESC | 15 |
| HUD - Hu antigen D (ELAVL4) | NM_021952 | | | |
| HUD-153 | | 153 | NGLRLQTKTIKVSYA | 15 |
| HUD-226 | | 226 | SRILVDQVTGVSRG | 15 |
| HUD-488 | | 488 | NGYRLGDRVLQVSFK | 15 |
| HUD-85 | | 85 | EFRSLFGSIGEIESC | 15 |
| HUR - Hu antigen R (ELAVL1) | NM_001419 | | | |
| HUR-106 | | 106 | NGLRLQSKTIKVSYA | 15 |
| HUR-35 | | 35 | TQDELRSLFSSIG | 13 |
| HUR-414 | | 414 | NGYRLGDKILQVSFK | 15 |
| HUR-186 | | 186 | QTTGLSRGVAFIRFD | 15 |
| HUR-179 | | 179 | NSRVLVDQTTGLSRG | 15 |
| CRMP5 - colapsin rec. | NM_020134 | | | |
| dihydropyrimidinase-like 5 (DPYSL5) | | | | |
| CRMP5-110 | | 110 | TKAALVGGTTMIIGH | 15 |
| CRMP5-660 | | 660 | RTPYLGDVAVVVHPG | 15 |
| CRMP5-418 | | 418 | LMSLLANDTLNIVAS | 15 |
| CRMP5-716 | | 716 | GMRDLHESSFSLSGS | 15 |
| CRMP5-642 | | 642 | VYKKLVQREKTLKVR | 15 |
| CRMP5-111 | | 111 | KAALVGGTTMIIGHV | 15 |
| CRMP5-558 | | 558 | EATKTISASTQVQGG | 15 |
| EXOSC1 hRrp46p | NM_016046 | | | |
| EXOSC1-98 | | 98 | KVSSINSRFAKVHIL | 15 |
| EXOSC1-185 | | 185 | SNYLLTTAENELGVV | 15 |
| EXOSC1-169 | | 169 | PGDIVLAKVISLGDA | 15 |
| EXOSC1-83 | | 83 | TESQLLPDVGAIVTC | 15 |
| EXOSC7 | NM_015004 | | | |
| EXOSC7-306 | | 306 | EACSLASLLVSVTSK | 15 |
| EXOSC7-349 | | 349 | VGKVLHASLQSVLHK | 15 |
| EXOSC7-176 | | 176 | HCWVLYVDVLLLECG | 15 |
| EXOSC5 | NM_020158 | | | |
| EXOSC5-255 | | 255 | ERKLLMSSTKGLYSD | 15 |
| EXOSC5-157 | | 157 | PRTSITVVLQVVSDA | 15 |
| EXOSC5-175 | | 175 | LACCLNAACMALVDA | 15 |
| EXOSC5-243 | | 243 | ARAVLTFALDSVERK | 15 |
| PGP 9.5 ubiquitin carboxyl-terminal | M30496 | | | |
| hydrolase UCH-L3 | | | | |
| PGP 9.5-263 | | 263 | SDETLLEDAIEVCKK | 15 |
| PGP 9.5-111 | | 111 | MKQTISNACGTIGLI | 15 |
| GAD2 - glutamate decarboxylase 2 | NM_000818 | | | |
| GAD2-714 | | 714 | RMSRLSKVAPVIKAR | 15 |
| GAD2-389 | | 389 | SHFSLKKGAAALGIG | 15 |
| GAD2-644 | | 644 | KCLELAEYLYNIIKN | 15 |
| GAD2-244 | | 244 | YFNQLSTGLDMVGLA | 15 |
| GAD2-328 | | 328 | PGGAISNMYAMMIAR | 15 |
| GAD2-152 | | 152 | TLAFLQDVMNILLQY | 15 |
| GAD2-783 | | 783 | DIDFLIEEIERLGQD | 15 |
| GAD2-304 | | 304 | VTLKKMREIIGWP | 13 |

TABLE 2
Disclosed are 51 peptide epitopes, from the set of 1,448 peptide epitopes
in Table 1, which were determined to be informative for distinguishing
between NSCLC, SCLC, and control. See Experimental.

| Number | Gene/epitope | peptide | mer |
|---|---|---|---|
| | TRP-2/4 | ANDPIFVVL | 9 |
| | HAGHL-237 | GHEHTLSNLEFAQKV | 15 |
| 14 | IQWD1-315 | SAENPVENHINITQS | 15 |
| 33 | KIAA0373-1107 | RKFAVIRHQQSLLYK | 15 |
| 38 | KIAA0373-1193 | MKKILAENSRKITVL | 15 |
| 88 | LOC401193-156 | EFLRSKKSSEEITQY | 15 |
| 103 | MSLN-186 | FSRITKANVDLLPRG | 15 |
| 108 | NACA-261 | AVRALKNNSNDIVNA | 15 |
| 113 | NISCH-805 | CIGYTATNQDFIQRL | 15 |
| 114 | NISCH-1764 | KTTGKMENYELIHSS | 15 |
| 117 | NISCH-1271 | THNCRNRNSFKLSRV | 15 |
| 122 | NISCH-1105 | RSCFAPQHMAMLCSP | 15 |
| 158 | RBMS1-108 | PYGKIVSTKAILDKT | 15 |
| 189 | ROCK2-1296 | HKQELTEKDATIASL | 15 |
| 272 | SDCCAG3-255 | SYDALKDENSKLRRK | 15 |
| 274 | SDCCAG3-462 | AEILKSIDRISEI | 13 |
| 278 | SDCCAG8-815 | ECCTLAKKLEQISQK | 15 |
| 377 | TP53-171 | YSPALNKMFCQLAKT | 15 |
| 409 | UTP14A-818 | IRDFLKEKREAVEAS | 15 |
| 411 | UTP14A-182 | TAQVLSKWDVVLKN | 15 |
| 454 | ZNF292-3415 | KKNNLENKNAKIVQI | 15 |
| 455 | ZNF292-1612 | TPQNLERQVNNLMTF | 15 |
| 458 | ZNF292-3154 | HKSDLPAFSAEVEEE | 15 |
| 501 | MELK-67 | NTLGSDLPRIKTE | 13 |
| 508 | MELK-241 | AAPELIQGKSYLGSE | 15 |
| 525 | NFRKB-1575 | SAVSLPSMNAAVSKT | 15 |
| 608 | AARS-1017 | TEEAIAKGIRRIVAV | 15 |
| 616 | ABL1-465 | NAVVLLYMATQISSA | 15 |
| 625 | ACAT2-488 | GCRILVTLLHTLERM | 15 |
| 780 | CTTNBP2-254 | EAQKLEDVMAKLEEE | 15 |
| 788 | DDX5-190 | TLSYLLPAIVHINHQ | 15 |
| 803 | DNAJA1-21 | TQEELKKAYRKLALK | 15 |
| 817 | DNM1L-3 | MEALIPVINKLQDV | 14 |
| 820 | DRCTNNB1A-588 | SSHGLAKTAATVF | 13 |
| 828 | ELKS-241 | KESKLSSSMNSIKTF | 15 |
| 843 | EXOSC10-883 | TTCLIATAVITLFNE | 15 |
| 884 | GOLGA2-1061 | THRALQGAMEKLQS | 14 |
| 965 | IQWD1-575 | EHLMLLEADNHVVNC | 15 |
| 972 | LIMS1-182 | KCHAIIDEQPLIFKN | 15 |
| 978 | LMNA-417 | LQKQLAAKEAKLRDL | 15 |
| 989 | MKRN1-483 | KQKLILKYKEAMSNK | 15 |
| 990 | NAP1L3-145 | AVRNRVQALRNI | 12 |
| 1042 | RBM25-978 | KRKHIKSLIEKIPTA | 15 |
| 1049 | RBPSUH-350 | IIRKVDKQTALLDA | 14 |
| 1050 | RBPSUH-236 | KKQSLKNADLCIASG | 15 |
| 1053 | SDCCAG1-232 | TLERLTEIVASAPKG | 15 |
| 1057 | SR-A1-1126 | RKVKLQSKVAVLIRE | 15 |
| 1115 | SOX1/17 | HPHAHPHNPQPMHRY | 15 |
| 1145 | NY-ESO-1/2 | GDADGPGGPGIPDGP | 15 |
| 1146 | NY-ESO-1/6 | PRGPHGGAASGLNGC | 15 |
| 1149 | SSX1/11 | SGPQNDGKQLHPPGK | 15 |

Tables 3-6 disclose the results of autoantibody profiling using 51 epitopes of Table 2 in NSCLC, SCLC and control samples. See Experimental.

TABLE 3
Classifier: NON-SMALL CELL LUNG CANCER SAMPLES as
training group
Number of markers in training
group: 1253
Method: Neural Network

| | Statistical | | Statistical | Plasma | Statistical |
|---|---|---|---|---|---|
| Plasma sample | match | Plasma sample | match | sample | match |
| NSCLC | 0% | Control | 0% | SCLC | 100% |
| NSCLC | 100% | Control | 0% | SCLC | 100% |
| NSCLC | 100% | Control | 0% | SCLC | 100% |
| NSCLC | 100% | Control | 0% | SCLC | 0% |
| NSCLC | 100% | Control | 0% | SCLC | 0% |
| NSCLC | 100% | Control | 0% | SCLC | 100% |
| NSCLC | 100% | Control | 0% | SCLC | 0% |
| NSCLC | 100% | Control | 0% | SCLC | 0% |
| NSCLC | 100% | Control | 0% | SCLC | 60% |
| NSCLC | 100% | Control | 0% | SCLC | 0% |
| NSCLC | 100% | Control | 0% | SCLC | 100% |
| NSCLC | 100% | Control | 0% | SCLC | 0% |
| NSCLC | 100% | Control | 0% | SCLC | 0% |
| NSCLC | 100% | Control | 0% | SCLC | 100% |
| NSCLC | 0% | Control | 0% | SCLC | 100% |
| NSCLC | 100% | Control | 0% | SCLC | 0% |
| NSCLC | 100% | Control | 0% | SCLC | 56% |
| NSCLC | 100% | Control | 100% | SCLC | 1% |
| NSCLC | 100% | Control | 0% | SCLC | 0% |
| NSCLC | 100% | Control | 7% | SCLC | 0% |
| NSCLC | 100% | Control | 0% | SCLC | 2% |
| NSCLC | 100% | Control | 0% | SCLC | 0% |
| NSCLC | 0% | Control | 0% | SCLC | 0% |
| NSCLC | 100% | Control | 0% | SCLC | 0% |
| NSCLC | 100% | Control | 0% | SCLC | 0% |
| NSCLC | 100% | Control | 65% | SCLC | 0% |
| NSCLC | 100% | Control | 0% | | |
| NSCLC | 100% | Control | 0% | | |
| NSCLC | 100% | Control | 0% | | |
| NSCLC | 0% | Control | 0% | | |
| NSCLC | 100% | Control | 9% | | |
| NSCLC | 100% | Control | 0% | | |
| NSCLC | 100% | Control | 0% | | |
| NSCLC | 0% | | | | |
| NSCLC | 100% | | | | |
| NSCLC | 100% | | | | |
| NSCLC | 0% | | | | |
| Mean | 0.837837838 | | 0.054848485 | | 0.315 |
| Standard Error | 0.061433251 | | 0.035571953 | | 0.08852857 |
| Median | 1 | | 0 | | 0 |
| Mode | 1 | | 0 | | 0 |
| Standard Deviation | 0.373683877 | | 0.204345315 | | 0.451408906 |
| Sample Variance | 0.13963964 | | 0.041757008 | | 0.20377 |
| Kurtosis | 1.745188398 | | 16.66992414 | | −1.295276226 |
| Skewness | −1.911470521 | | 4.095015871 | | 0.831444585 |
| Range | 1 | | 1 | | 1 |
| Minimum | 0 | | 0 | | 0 |
| Maximum | 1 | | 1 | | 1 |
| Sum | 31 | | 1.81 | | 8.19 |
| Count | 37 | | 33 | | 26 |

TABLE 4
Method:
Support Vector Machine: Radial Base Function kernel.

| Plasma | | | | | |
|---|---|---|---|---|---|
| sample | Statistical match | Plasma sample | Statistical match | Plasma sample | Statistical match |
| NSCLC | 81% | Control | 41 % | SCLC | 35% |
| NSCLC | 98% | Control | 1% | SCLC | 58% |
| NSCLC | 98% | Control | 0% | SCLC | 30% |
| NSCLC | 100% | Control | 3% | SCLC | 6% |
| NSCLC | 101% | Control | −2% | SCLC | 32% |
| NSCLC | 100% | Control | −3% | SCLC | 91% |
| NSCLC | 86% | Control | 1% | SCLC | 13% |
| NSCLC | 102% | Control | 2% | SCLC | 4% |
| NSCLC | 90% | Control | 1% | SCLC | 43% |
| NSCLC | 88% | Control | 2% | SCLC | 21% |
| NSCLC | 90% | Control | −2% | SCLC | 4% |
| NSCLC | 66% | Control | −21% | SCLC | 4% |
| NSCLC | 100% | Control | 2% | SCLC | 4% |
| NSCLC | 97% | Control | 4% | SCLC | 43% |
| NSCLC | 92% | Control | −12% | SCLC | 22% |
| NSCLC | 78% | Control | −20% | SCLC | 19% |
| NSCLC | 92% | Control | 0% | SCLC | 3% |
| NSCLC | 42% | Control | 1% | SCLC | 5% |
| NSCLC | 102% | Control | −1% | SCLC | 5% |
| NSCLC | 100% | Control | 5% | SCLC | 2% |
| NSCLC | 98% | Control | −2% | SCLC | 12% |
| NSCLC | 98% | Control | −6% | SCLC | 13% |
| NSCLC | 59% | Control | 1% | SCLC | 3% |
| NSCLC | 36% | Control | −5% | SCLC | − 2% |
| NSCLC | 97% | Control | 23% | SCLC | 3% |
| NSCLC | 90% | Control | 4% | SCLC | −3% |
| NSCLC | 97% | Control | 1% | | |
| NSCLC | 87% | Control | −9% | | |
| NSCLC | 97% | Control | −15% | | |
| NSCLC | 23% | Control | 1% | | |
| NSCLC | 82% | Control | 1% | | |
| NSCLC | 100% | Control | 3% | | |
| NSCLC | 81% | Control | 1% | | |
| NSCLC | 101% | | | | |
| NSCLC | 83% | | | | |
| NSCLC | 60% | | | | |
| NSCLC | 56% | | | | |
| Mean | 0.850810811 | | −0.0003125 | | 0.180769231 |
| Standard Error | 0.032816668 | | 0.019257824 | | 0.042891359 |
| Median | 0.92 | | 0.01 | | 0.09 |
| Mode | 1 | | 0.01 | | 0.04 |
| Standard Deviation | 0.199615998 | | 0.108938704 | | 0.218703874 |
| Sample Variance | 0.039846547 | | 0.011867641 | | 0.047831385 |
| Kurtosis | 2.220723288 | | 6.551736654 | | 3.841127046 |
| Skewness | −1.669600142 | | 1.551257739 | | 1.830688658 |
| Range | 0.79 | | 0.62 | | 0.94 |
| Minimum | 0.23 | | −0.21 | | −0.03 |
| Maximum | 1.02 | | 0.41 | | 0.91 |
| Sum | 31.48 | | −0.01 | | 4.7 |
| Count | 37 | | 32 | | 26 |

TABLE 5
Classifier of the Arrays: NSCLC samples on 50 marker set
Method: Support Vector Machine: Radial Base Function kernel.

| Plasma sample | Statistical match | Plasma sample | Statistical match | Plasma sample | Statistical match |
|---|---|---|---|---|---|
| NSCLC | 102% | Control | 51% | SCLC | 3% |
| NSCLC | 89% | Control | −2% | SCLC | 2% |
| NSCLC | 85% | Control | 12% | SCLC | 15% |
| NSCLC | 98% | Control | −5% | SCLC | 30% |
| NSCLC | 76% | Control | −14% | SCLC | 53% |
| NSCLC | 102% | Control | −2% | SCLC | 88% |
| NSCLC | 94% | Control | 0% | SCLC | −3% |
| NSCLC | 99% | Control | 10% | SCLC | 4% |
| NSCLC | 77% | Control | −6% | SCLC | 20% |
| NSCLC | 82% | Control | 4% | SCLC | 17% |
| NSCLC | 71% | Control | −1% | SCLC | 3% |
| NSCLC | 62% | Control | −22% | SCLC | 4% |
| NSCLC | 63% | Control | 5% | SCLC | 2% |
| NSCLC | 57% | Control | 2% | SCLC | 21% |
| NSCLC | 101% | Control | 2% | SCLC | 3% |
| NSCLC | 100% | Control | −30% | SCLC | 11% |
| NSCLC | 64% | Control | 4% | SCLC | 0% |
| NSCLC | 11% | Control | −13% | SCLC | 0% |
| NSCLC | 101% | Control | −15% | SCLC | 2% |
| NSCLC | 97% | Control | 3% | SCLC | 7% |
| NSCLC | 97% | Control | −4% | SCLC | 6% |
| NSCLC | 82% | Control | −14% | SCLC | −1% |
| NSCLC | 68% | Control | 0% | SCLC | 4% |
| NSCLC | 34% | Control | −17% | SCLC | 10% |
| NSCLC | 98% | Control | 20% | SCLC | −2% |
| NSCLC | 79% | Control | 34% | SCLC | 2% |
| NSCLC | 76% | Control | 3% | | |
| NSCLC | 98% | Control | −15% | | |
| NSCLC | 85% | Control | −1% | | |
| NSCLC | 17% | Control | 3% | | |
| NSCLC | 43% | Control | −32% | | |
| NSCLC | 71% | Control | 4% | | |
| NSCLC | 45% | Control | −4% | | |
| NSCLC | 82% | | | | |
| NSCLC | 98% | | | | |
| NSCLC | 26% | | | | |
| NSCLC | 75% | | | | |
| Mean | 0.758108 | | −0.012121212 | | 0.115769231 |
| Standard Error | 0.040918 | | 0.027987272 | | 0.03869873 |
| Median | 0.82 | | −0.01 | | 0.04 |
| Mode | 0.98 | | 0.04 | | 0.02 |
| Standard Deviation | 0.248896 | | 0.16077464 | | 0.19732558 |
| Sample Variance | 0.061949 | | 0.025848485 | | 0.038937385 |
| Kurtosis | 0.581168 | | 3.018160625 | | 9.147145282 |
| Skewness | −1.1099 | | 0.984452432 | | 2.863009047 |
| Range | 0.91 | | 0.83 | | 0.91 |
| Minimum | 0.11 | | −0.32 | | −0.03 |
| Maximum | 1.02 | | 0.51 | | 0.88 |
| Sum | 28.05 | | −0.4 | | 3.01 |
| Count | 37 | | 33 | | 26 |

TABLE 6
Classifier: NON-SMALL CELL LUNG
CANCER SAMPLES as training group
Number of markers in training group:
entire peptide library

| | | |
|---|---|---|
| NSCLC | NON-CANCER | SCLC |
| Statistical | Control | Statistical |
| match | Statistical match | match |

METHOD 1
Method: Neural Network

| | | | |
|---|---|---|---|
| Mean | 0.837837838 | 0.054848485 | 0.315 |
| Standard Error | 0.061433251 | 0.035571953 | 0.08852857 |
| number of samples | 37 | 33 | 26 |

METHOD 2
Support Vector Machine: Radial Base Function kernel

| | | |
|---|---|---|
| 0.850810811 | −0.0003125 | 0.180769 |
| 0.032816668 | 0.019257824 | 0.042891 |
| 37 | 32 | 26 |

Classifier: NSCLC samples as training group
Number of markers: 50 peptides
Support Vector Machine: Radial Base Function kernel

| | | | |
|---|---|---|---|
| Mean | 0.758108108 | −0.012121212 | 0.115769231 |
| Standard Error | 0.040918211 | 0.027987272 | 0.03869873 |
| number of samples | 37 | 33 | 26 |

Abbreviations:
NSCLC—non-small cell lung cancer
SCLC—small cell lung cancer

Table 7 discloses additional epitopes, corresponding to differentiation antigens, that may be Used for autoantibody profiling

Differentiation antigens

| | |
|---|---|
| CEA | YLSGANLNL |
| | IMIGVLVGV |
| | HLFGYSWYK |
| | YACFVSNLATGRNNS |
| | LWWVNNQSLPVSP |
| gp100/Pmel17 | KTWGQYWQV |
| | AMLGTHTMEV |
| | ITDQVPFSV |
| | YLEPGPVTA |
| | LLDGTATLRL |
| | VLYRYGSFSV |
| | SLADTNSLAV |
| | RLMKQDFSV |
| | RLPRIFCSC |
| | LIYRRRLMK |
| | ALLAVGATK |
| | IALNFPGSQK |
| | ALNFPGSQK |
| | VYFFLPDHL |
| | RTKQLYPEW |
| | HTMEVTVYHR |
| | VPLDCVLYRY |
| | SNDGPTLI |
| Kallikrein4 | SVSESDTIRSISIAS |
| | LLANGRMPTVLQCVN |
| | RMPTVLQCVNVSVVS |
| mammaglobin-A | PLLENVISK |
| Melan-A/MART-1 | EAAGIGILTV |
| | ILTVILGVL |
| | AEEAAGIGILT |
| | RNGYRALMDKSLHVGTQCALTRR |
| PSA | FLTPKKLQCV |
| | VISNDVCAQV |
| TRP-1/gp75 | MSLQRQFLR |
| | SLPYWNFATG |
| TRP-2 | SVYDFFVWL |
| | TLDSQVMSL |
| | LLGPGRPYR |
| | ANDPIFVVL |
| | ALPYWNFATG |
| tyrosinase | KCDICTDEY |
| | SSDYVIPIGTY |
| | MLLAVLYCL |
| | CLLWSFQTSA |
| | YMDGTMSQV |
| | AFLPWHRLF |
| | TPRLPSSADVEF |
| | LPSSADVEF |
| | SEIWRDIDFd |
| | QNILLSNAPLGPQFP |
| | SYLQDSDPDSFQD |
| | FLLHHAFVDSIFEQWLQRHRP |

Table 8 discloses addtional epitopes, corresponding to antigens overexpressed in tumors, That may be used for autoantibody profiling.

| ANTIGENS OVEREXPRESSED IN TUMORS | |
|---|---|
| adipophilin | SVASTITGV |
| CPSF | KVHPVIWSL |
| | LMLQNALTTM |
| EphA3 | DVTFNIICKKCG |
| G250/MN/CAIX | HLSTAFARV |
| HER-2/neu | KIFGSLAFL |
| | IISAVVGIL |
| | ALCRWGLLL |
| | ILHNGAYSL |
| | RLLQETELV |
| | VVLGVVFGI |
| | YMIMVKCWMI |
| | HLYQGCQVV |
| | YLVPQQGFFC |
| | PLQPEQLQV |
| | TLEEITGYL |
| | ALIHHNTHL |
| | PLTSIISAV |
| | VLRENTSPK |
| Intestinalcarboxylesterase | SPRWWPTCL |
| alpha-foetoprotein | GVALQTMKQ |
| M-CSF | LPAVVGLSPGEQEY |
| MUC1 | STAPPVHNV |
| | LLLLTVLTV |
| | PGSTAPPAHGVT |
| p53 | LLGRNSFEV |
| | RMPEAAPPV |
| | SQKTYQGSY |
| PRAME | VLDGLDVLL |
| | SLYSFPEPEA |
| | ALYVDSLFFL |
| | SLLQHLIGL |
| | LYVDSLFFL |
| PSMA | NYARTEDFF |
| RAGE-1 | SPSSNRIRNT |
| RU2AS | LPRWPPPQL |
| survivin | ELTLGEFLKL |
| Telomerase | ILAKFLHWL |
| | RLVDDFLLV |
| | RPGLLGASVLGLDDI |
| | LTDLQPYMRQFVAHL |
| WT1 | CMTWNQMNL |

Table 9 discloses addtional epitopes corresponding to antigens expresses in multiple tumor Types, that may be used for autoantibody profiling

SHARED TUMOR SPECIFIC ANTIGENS

| | |
|---|---|
| BAGE-1 | AARAVFLAL |
| GAGE-1,2,8 | YRPRPRRY |
| GAGE-3,4,5,6,7 | YYWPRPRRY |
| GnTVf | VLPDVFIRCV |
| HERV-K-MEL | MLAVISCAV |
| LAGE-1 | MLMAQEALAFL |
| | SLLMWITQC |
| | LAAQERRVPR |
| | SLLMWITQCFLPVF |
| | QGAMLAAQERRVPRAAEVPR |
| | AADHRQLQLSISSCLQQL |
| | CLSRRPWKRSWSAGSCPGMPHL |
| | ILSRDAAPLPRPG |
| MAGE-A1 | EADPTGHSY |
| | SLFRAVITK |
| | EVYDGREHSA |
| | RVRFFFPSL |
| | EADPTGHSY |
| | REPVTKAEML |
| | DPARYEFLW |
| | ITKKVADLVGF |
| | SAFPTTINF |
| | SAYGEPRKL |
| | LLKYRAREPVTKAE |
| | EYVIKVSARVRF |
| MAGE-A2 | YLQLVFGIEV |
| | EYLQLVFGI |
| | REPVTKAEML |
| | EGDCAPEEK |
| | LLKYRAREPVTKAE |
| MAGE-A3 | EVDPIGHLY |
| | FLWGPRALV |
| | KVAELVHFL |
| | TFPDLESEF |
| | MEVDPIGHLY |
| | EVDPIGHLY |
| | REPVTKAEML |
| | AELVHFLLL |
| | MEVDPIGHLY |
| | WQYFFPVIF |
| | EGDCAPEEK |
| | KKLLTQHFVQENYLEY |
| | ACYEFLWGPRALVETS |
| | VIFSKASSSLQL |
| | GDNQIMPKAGLLIIV |
| | TSYVKVLHHMVKISG |
| | AELVHFLLLKYRAR |
| | LLKYRAREPVTKAE |
| MAGE-A4 | EVDPASNTY |
| | GVYDGREHTV |
| | SESLKMIF |
| MAGE-A6 | MVKISGGPR |
| | EVDPIGHVY |
| | REPVTKAEML |
| | EGDCAPEEK |
| | LLKYRAREPVTKAE |
| MAGE-A10 | GLYDGMEHL |
| | DPARYEFLW |
| MAGE-A12 | FLWGPRALV |
| | VRIGHLYIL |
| | EGDCAPEEK |
| | AELVHFLLLKYRAR |
| MAGE-C2 | LLFGLALIEV |
| | ALKDVEERV |
| NA-88 | QGQHFLQKV |
| NY-ESO-1/LAGE-2 | SLLMWITQC |
| | ASGPGGGAPR |
| | LAAQERRVPR |
| | MPFATPMEA |
| | MPFATPMEA |
| | LAMPFATPM |
| | ARGPESRLL |
| | SLLMWITQCFLPVF |
| | QGAMLAAQERRVPRAAEVPR |
| | PGVLLKEFTVSGNILTIRLT |
| | VLLKEFTVSG |
| | AADHRQLQLSISSCLQQL |
| | PGVLLKEFTVSGNILTIRLTAADHR |
| Sp17 | ILDSSEEDK |
| SSX-2 | KASEKIFYV |
| | EKIQKAFDDIAKYFSK |
| | KIFYVYMKRKYEAM |
| TRP2-INT2g | EVISCKLIKR |

Table 10 discloses additional epitopes, corresponding to tumor antigens that arise through Mutation, that may be used for autoantibody profiling.

| Tumor antigens resulting from | |
|---|---|
| mutations | |
| alpha-actinin-4 | FIASNGVKLV |
| BCR-ABLfusionprotein(b3a2) | SSKALQRPV |
| | GFKQSSKAL |
| | ATGFKQSSKALQRPVAS |
| CASP-8 | FPSDSWCYF |
| beta-catenin | SYLDSGIHF |
| Cdc27 | FSWAMDLDPKGA |
| CDK4 | ACDPHSGHFV |
| CDKN2A | AVCPWTWLR |
| COA-1f | TLYQDDTLTLQAAG |
| dek-canfusionprotein | TMKQICKKEIRRLHQY |
| Elongationfactor2 | ETVSEQSNV |
| ETV6-AML1fusionprotein | RIAECILGM |
| | IGRIAECILGMNPSR |
| LDLR- | WRRAPAPGA |
| fucosyltransferaseASfusionprotein | |
| | PVTWRRAPA |
| hsp70-2 | SLFEGIDIYT |
| KIAAO205 | AEPINIQTW |
| MART2 | FLEGNEVGKTY |
| MUM-1f | EEKLIVVLF |
| MUM-2 | SELFRSGLDSY |
| | FRSGLDSYV |
| MUM-3 | EAFIQPITR |
| neo-PAP | RVIKNSIRLTL |
| MyosinclassI | KINKNPKYK |
| OS-9g | KELEGILLL |
| pml-RARalphafusionprotein | NSNHVASGAGEAAIETQSSSS |
| | EEIV |
| PTPRK | PYYFAAELPPRNLPEP |
| K-ras | VVVGAVGVG |
| N-ras | ILDTAGREEY |
| TriosephosphateIsomerase | GELIGILNAAKVPAD |

Table 11 discloses are 25 preferred lung cancer deteministic epitopes from the set of 1,448 Peptide epitopes in Table 1. See Experimental.

| | | | |
|---|---|---|---|
| 1 | GRINA-398 | TCFLAVDTQLLLGNK | 15 |
| 2 | AP1G21020 | LFRILNPNKAPLRLK | 15 |
| 14 | IQWD1-315 | SAENPVENHINITQS | 15 |
| 33 | KIAA0373-1107 | RKFAVIRHQQSLLYK | 15 |
| 38 | KIAA0373-1193 | MKKILAENSRKITVL | 15 |
| 88 | LOC401193-156 | EFLRSKKSSEEITQY | 15 |
| 103 | MSLN-186 | FSRITKANVDLLPRG | 15 |
| 108 | NACA-261 | AVRALKNNSNDIVNA | 15 |
| 114 | NISCH-1764 | KTTGKMENYELIHSS | 15 |
| 117 | NISCH-1271 | THNCRNRNSFKLSRV | 15 |
| 122 | NISCH-1105 | RSCFAPQHMAMLCSP | 15 |
| 158 | RBMS1-108 | PYGKIVSTKAILDKT | 15 |
| 274 | SDCCAG3-462 | AEILKSIDRISEI | 13 |
| 411 | UTP14A-182 | TAQVLSKWDPVVLKN | 15 |
| 454 | ZNF292-3415 | KKNNLENKNAKIVQI | 15 |
| 455 | ZNF292-1612 | TPQNLERQVNNLMTF | 15 |
| 525 | NFRKB-1575 | SAVSLPSMNAAVSKT | 15 |
| 608 | AARS-1017 | TEEAIAKGIRRIVAV | 15 |
| 616 | ABL1-465 | NAVVLLYMATQISSA | 15 |
| 828 | ELKS-241 | KESKLSSSMNSIKTF | 15 |
| 965 | IQWD1-575 | EHLMLLEADNHVVNC | 15 |
| 972 | LIMS1-182 | KCHAIIDEQPLIFKN | 15 |
| 1050 | RBPSUH-236 | KKQSLKNADLCIASG | 15 |
| 1057 | SR-A1-1126 | RKVKLQSKVAVLIRE | 15 |
| 1146 | NY-ESO-1/6 | PRGPHGGAASGLNGC | 15 |

Table 12 discloses the results of autoantibody profiling using 25 epitopes of Table 11 in NSCLC control samples. See Experimental.

Support Vector Machine: Radial Base Function kernel
Layer: RawData
Subset: Complete set

Statistical match to NSCLC Classifier

| | NSCLC | CONTROL |
|---|---|---|
| Mean | 0.948275862 | 0.124516129 |
| Standard Error | 0.020541134 | 0.037884484 |

t-Test: Two-Sample Assuming Equal Variances

| | Variable 1 | Variable 2 |
|---|---|---|
| Mean | 0.948275862 | 0.124516129 |
| Variance | 0.012236207 | 0.044492258 |
| Observations | 29 | 31 |
| Pooled Variance | 0.028920371 | |
| Hypothesized Mean Difference | 0 | |
| df | 58 | |
| t Stat | 18.75006802 | |
| P(T < = t) one-tail | 1.35315E−26 | |
| t Critical one-tail | 1.671552763 | |
| P(T < = t) two-tail | 2.70629E−26 | |
| t Critical two-tail | 2.001717468 | |

NSCLC = NON-SMALL LUNG CANCER
We tested an array that contained 25 of our best markers (the ones that scored the best among the entire peptide library)
We tested these 25-marker arrays with 29 NSCLC and 31 non-cancer control markers
We carried out the pattern recognition using Support Vector Machine (available in GeneMath XT bioinformatics package)

### EXPERIMENTAL

We have carried out pilot studies on breast and lung cancer. In our breast cancer study, we determined the serum aAB composition in 16 breast cancer patients and 16 gender-matched non-cancer control individuals. The lung cancer study was carried out as a comparative study on NSCLC and SCLC sera in order to detect differences between these two predominant types of lung cancer. Both of these pilot studies were carried out simultaneously with the same set of epitopes. This set included 428 different epitopes representing 135 different proteins. The informative epitopes were sorted into two groups based on an increased/decreased (I/D) signal dichotomy. Briefly, we carried out a cancer vs. non-cancer comparison for breast cancer, and an NSCLC vs. SCLC for lung cancer using the neighborhood analysis. This method, adopted from large-scale gene-expression studies (Golub et al., Science (1999) 286:531-7) identifies informative peptide epitopes. Informative epitopes are the epitopes that produce a significantly different signal in one group of patient sera compared with another group of patient sera.

### Breast Cancer: Informative Epitopes

The breast cancer pilot study produced a set of 27 informative epitopes exhibiting an increased/decreased (I/D) dichotomy (FIG. 2). Intriguingly, the subset of epitopes that produced a decreased signal was greater than the subset of epitopes which produced an increased signal in breast cancer compared with non-cancer control. For both subsets of informative epitopes, the highly significant p-values were determined in the EB vs. EC comparison (FIG. 2).

The I/D-dichotomy for informative breast cancer epitopes is significantly disproportional. Determined on unsorted informative epitopes, EB was significantly smaller than EC (22±0.8 vs. 30±1.3, respectively; p=0.00000183). Thus, as demonstrated by informative breast cancer epitopes, the capacity of peptide epitopes to produce an in vitro immune reaction with serum aAB is smaller in breast cancer compared with non-cancer control (FIG. 2). We interpret this result as an indication that breast cancer sera contain either lower titer aAB or lower affinity aAB than control sera. In fact, we hypothesize that this “fading” of the “in vitro immune reaction” in breast cancer points to a weakened B-cell immunity. Nevertheless, we believe that also the anti-tumor humoral immune response is manifest in breast cancer because we detected a sub-set of informative epitopes that produced a significantly increased in vitro immune reaction in breast cancer sera (FIG. 2).

Lung Cancer: NSCLC vs. SCLC: Informative Epitopes

The lung cancer pilot study produced 28 informative epitopes that characterize the serum aAB difference between NSCLC and SCLC. Similar to the informative breast cancer epitopes, the informative lung cancer epitopes exhibited a significantly disproportional I/D-dichotomy (FIG. 3). Specifically, ES was significantly smaller than EN (28.4±1.0 vs. 32.5±0.9; p=0.006). Considering also our breast cancer study, and the published data about cancer survival, the following hypothesis can be put forward: Decreased average informative epitope strength [E] in breast cancer and SCLC indicate a compromised immune status of breast cancer and SCLC patients compared with their reference groups. This weakened immune status explains poorer survival in breast cancer and SCLC relative to non-cancer controls and NSCLC patients, respectively. As demonstrated by the Mayo Lung Project, the median survival is shorter and the 5-year survival poorer in SCLC compared with NSCLC (Marcus et al., J Natl Cancer Inst. (2000) 92:1308-16). Furthermore, in view of the above hypothesis, it is reasonable that a smaller difference emerged between ES and EN compared with EB and EC because non-cancer individuals generally have a better life expectancy than cancer patients.

### Epitope Microarray Reveals Higher Order Among Informative Cancer Epitopes: (i) Overlapping Informative Epitopes

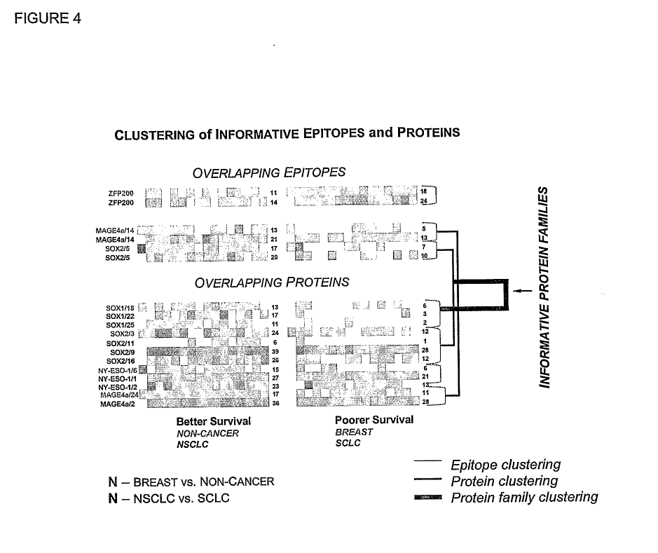

The two above pilot studies revealed an overlap (FIG. 4). We detected three epitopes that were informative for both breast and lung cancer (FIG. 4). Intriguingly, all three of these overlapping epitopes exhibited the same I/D-dichotomy in regard to the published knowledge about cancer survival. Specifically, ZFP-200 produced an increased signal in both breast cancer and SCLC relative to the non-cancer control and NSCLC, respectively; MAGE4a/14 and SOX2/5 produced a decreased signal in breast cancer and SCLC relative to the non-cancer control and NSCLC.

### (ii) Overlapping Informative Proteins

We also detected informative epitopes that did not overlap but represented the same protein (FIG. 4). Non-overlapping epitopes from four proteins, MAGE4a, NY-ESO, SOX-1 and SOX-2, produced an informative signal for both breast and lung cancer. The I/D-dichotomy of all four of these proteins in regard to the published cancer survival data (Marcus et al., J Natl Cancer Inst. (2000) 92:1308-16) was the same in that they all exhibited a decreased in vitro immune reactivity in the poorer survival group (FIG. 4). Thus, clustering of both informative epitopes and proteins to reveal aAB associations between cancer types, and potentially common pathogenic mechanisms, appears to be possible using an epitope microarray.

### Epitope Validation

With our cancer epitope microarrays, we have focused on (1) transcription factors expressed in embryonal tissues (Gure et al. supra; Chen et al., (1997) supra), (2) proteins known to trigger B-cell response in cancer (Tan, supra, Lubin, supra), and (3) proteins with embryo/testis/tumor specificity known to activate tumor specific cytolytic T-cells (Van Der Bruggen et al., Immunol Rev. (2002) 188:51-64; Boon et al., Annu Rev Immunol. (1994) 12:337-65). As our pilot studies indicate, this approach appears to bear fruit in that the informative epitopes for both breast and lung cancer include members of the SOX-family (embryo specific transcription factor), p53, members of IMP and HuD-family (known inducers of B-cell response in cancer), and tumor/testis/cancer proteins such as members of MAGE and NY-ESO family (FIGS. 2-4).

### Epitope Signal Analysis

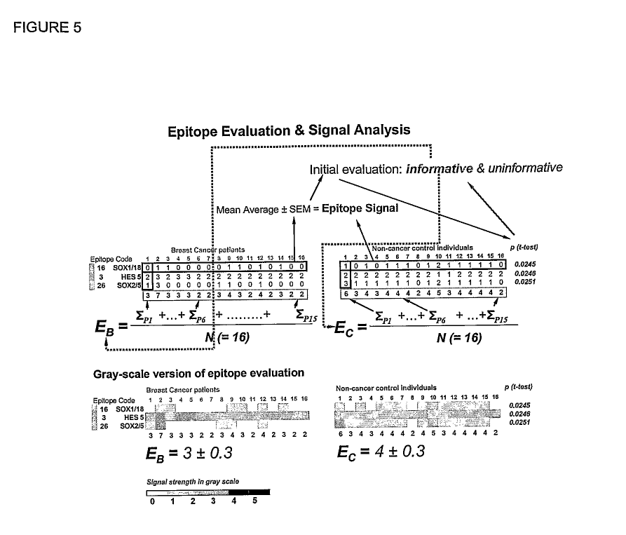

We used the neighborhood analysis (Golub et al., supra) in order to determine informative epitopes. We included both signal frequency and intensity in data analysis. Mean average ±SEM of signal intensity per a specific epitope in a group is referred to as an epitope signal. In order to evaluate epitopes, we carried out a two-sided Student t-test assuming equal variance (FIG. 5) on epitope signals. All epitopes that produce a significantly different epitope signal in a two-way comparison were considered informative epitopes. The example in FIG. 5 illustrates the evaluation of epitopes. In addition to epitope signal, the following endpoints were calculated and evaluated in data analysis:

ΣP—composite signal strength for all informative epitopes per an individual test subject;

E—Average Informative Epitope Strength per group of patients;

E=[ΣP1+ . . . +ΣPn/N]±SEM, where N denotes a number of patients in a group (FIG. 5). This parameter is calculated for both unsorted and sorted data.

### Signal Detection and Quantification

Our preliminary comparative experiments on alkaline phosphatase-(“AP”) based colorimetry and Cy3-based fluorimetry indicate that the signal over background ratio is up to an order of magnitude greater when Cy3 in place of AP is used (data not shown). This result is in agreement with previous studies indicating that fluorescence-based labeling produces a superior dynamic signal range over traditional color-producing labeling (Boon et al., supra).

Our existing, colorimetry-based data have the maximum range of 3 in 99% cases. Cy3-fluorescence-based experiments are done using neighborhood analysis in order decrease underestimates and overestimates of epitope importance based on colorimetric data. Somewhat different informative epitope sets may emerge. Because of greater sensitivity, the smaller quantities of sera required per assay are envisioned as a very relevant benefit of the fluorimetry-based visualization platform; a benefit that will increase in importance as the density of epitopes on the microarray increases.

### Data Normalization

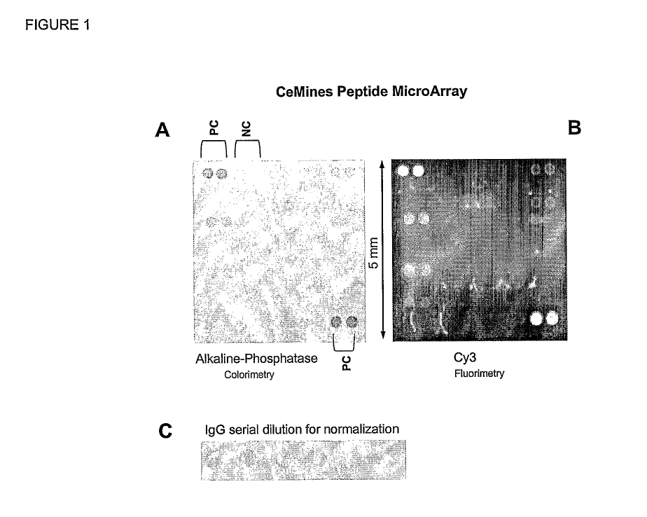

As depicted in FIG. 1, signal quantification and normalization is improved by implementing an internal control that is based on serial dilutions of human IgG. This internal control enables a more accurate normalization of each one of the individual peptide:aAB interactions as compared to single-concentration based signal quantification. As a result, the individual peptide epitope/aAB-binding activities may be expressed as equivalents of immunoreactivity of x-amount of human IgG. Introducing this specific normalization feature will improve the compatibility of the data from different experiments and test sites.

### Data Analysis

Epitopes that produce the greatest variance in the t-test are sorted in order determine the value of the most deviating epitopes. As our preliminary data indicate, approximately 1% of all individual peptide/autoantibody binding reactions produce a very strong signal, which in some cases exceeds even the positive control (data not shown). These rare, very strong signals may represent the cases in which a certain epitope detects a specific high-affinity anti-tumor serum aAB. Cy3-based fluorimetric detection is validated because it produces a greater dynamic range for the epitope microarray. Use of Cy3 reveals epitopes that identify high titer and high affinity anti-tumor serum aAB. Both colorimetry- and fluorimetry-produced data are analyzed and cross-validated. Cross-validation includes both p-value and variance-based analyses.

Power of Individual aABs and aAB Patterns

The system used determines (1) the individual diagnostic powers of each one of the informative epitopes, and (2) validates the diagnostic power of various combinations of informative epitopes (aAB patterns). The former can be achieved using the principles of “weighted votes” described by Golub et al., supra, whereas the latter can be accomplished using various pattern recognition algorithms, and then validating the resulting patterns individually. Briefly, in order to elucidate the diagnostic power of individual epitopes, a system of “weighted votes” may be used. In this type of system, the capacity of an informative epitope to predict a certain tumor is dependent on (1) its ability to alter the diagnostic power of a group of informative epitopes, and (2) to predict a tumor class in a blinded study. Specifically, the greater the capacity of an individual epitope to alter the diagnostic power of a group of epitopes, the more likely this epitope is to predict a certain tumor. The epitopes with the greatest individual predictive power will also be the most valuable markers in a blinded study. Because of enormous genetic complexity of cancer, and the variability of immune responses and antigen presentation, the diagnostic utility of various aAB patterns surpasses the diagnostic utility of individual epitopes.

### Different Epitopes Corresponding to Same Antigen Have Different Diagnostic Values

Proteins as antigens carry large number of epitopes that are not equally immunogenic and are not equally presented by antigen presenting and tumor cells.

For example from twenty-two KIA0373 epitopes, only two (KIAA0373-1107-RKFAVIRHQQSLLYK; and KIAA0373-1193-MKKILAENSRKITVL) exhibit consistent autoantibody binding activity and strong diagnostic value for NSCLC. Similar distinctions in diagnostic value between individual epitopes are observed for NISCH, SDCCAG3, ZNF292, RBPSUH and many other proteins.

In conclusion, our analysis has demonstrated that different epitopes from the same protein antigen may have different and even opposite diagnostic values. For example antibodies recognizing epitope SOX3/7 (peptide—PAMYSLLETELKNPV) are present and characteristic for NSCLC and epitope SOX3/14 (peptide—DEAKRLRAVHMKEYP) is characteristic for SCLC.

### Large Scale Autoantibody Profiling of Lung Cancer Patients: Diagnostic Value of Autoantibody Patterns

This study has three groups of patients:

1. healthy patients with history of heavy smoking (32 patients)

2. non small cell lung cancer patients (36 patients)

3. small cell lung cancer patients (26 patients)

Blood serum from all study individuals was analyzed using a peptide epitope array with 1,253 of the 1,448 peptide epitopes disclosed in Table 1.

Array images were analyzed using Array-Pro Analyzer (Media Cybernetics) and image data were analyzed using GeneMaths XT (Applied Maths) to obtain patterns of autoantibody binding activities that are characteristic for cancer patients and can be used as diagnostic tools. (Tables 3-6)

Analysis using Neural Networks and Support Vector Machine software demonstrated that discrete groups of autoantibodies are present in each patient category. In this specific set of study individuals, non small cell cancer patients can be grouped together with 83-85% specificity, whereas control patients belong to this group with less than 5% probability. (Tables 3-6)

### Autoantibody Profiling of Lung Cancer Patients: Lung Cancer Deterministic Peptides

A peptide array containing 25 of the most informative epitopes (Table 11) was used with the samples described above. This array contained the peptides that produced the best discrimination between non-small cell lung cancer (NSCLC) and control samples in the large-scale screening with 1,253 of the 1,448 peptide epitopes disclosed in Table 1. We refer to these as ‘lung cancer deterministic peptides’, which can be used as a highly accurate set of lung cancer diagnostic epitopes. We used Support Vector Machine as a pattern recognition algorithm. First, we used all of the NSCLC samples to compose a classifier and then we applied this classifier on both NSCLC and control samples. The average similarity of an NSCLC sample to the NSCLC classifier turned out to be ˜95%, and that of a control sample, 12.5%. (Table 12)

### Detection of Auto-antibodies: Peptide Microarray Protocol Using Nitrocellulose Pads on Coverslips

Microarray slides are commercially available, for example from Schleicher & Schuell. The protocol is a follows:

1. Blocking with Superblock, TBS based (pH 7.4), (Pierce Cat# 37535), 0.05% Tween 20 for 1 h at room temperature. Use 100-150 μl of blocking solution per well (16 pad slides)

2. Wash twice with TBS, pH 7.4 and 0.05% Tween 20 at room temperature 2 min each wash. Each wash 150 μl.

3. Dilute serum 1:15 with TBS, pH 7.4 containing Superblock diluted 1:10 and 0.05% Tween 20.

4. Incubate array with 150 μl of diluted serum overnight at +4° C. (minimum 16 hours).

5. Wash 5 times using TBS, pH 7.4 containing 0.05% Tween 20 at room temperature 5 min each wash. Each wash 150 μl.

6. Incubate with secondary antibody (alkaline phosphatase conjugated anti human IgA, IgM, IgG; ChemiconAP120A, lot 23091469) diluted 1:3000 with TBS, pH 7.4 containing Superblock diluted 1:10 and 0.05% Tween 20 for 1 hour at room temperature. Volume 150 μl.

7. Wash 5 times using TBS, pH 7.4 containing 0.05% Tween 20 at room temperature 5 min each wash. Each wash 150 μl.

8. Visualize auto-antibody binding using alkaline phosphatase substrate (Pierce 1-Step NBT/BCIP, product # 34042). It will take 15-30 minutes to see reaction products. Do not over incubate. Long incubation time will result in high background.

9. Stop reaction by rinsing with water

10. Dry slides and analyze.

Peptide Printing Protocol using Perkin Elmer Piezzo Arrayer

Preparation:

0.1% Tween in PBS Buffer

HPLC Grade Water

50 mM NaOH

Repel-Silane ES

HPLC Methanol

Method:

Before any run do the following:

Prime the tips using the Prime Utility;

Clean the tips with 50 mM NaOH, using the advance NaOH cleaning utility;

3) Prime the tips using the Prime Utility;

4) Silanate the tips using the Silanate Utility, the first four wells should be filled with 100% HPLC Grade Methanol; protein precipitation should not occur due to the NaOH cleaning; the last four wells will contain the Repel-Silane ES solution;

5) Prime the tips using the Prime Utility;

6) Tune the tips using the Tuning Utility;

7) Do a Standard Wash.

Setting up the protocol:

1) The Wash settings tab should be set to the following: syringe wash volume is 400 μl, Peripump on time is 10 seconds, and Sonication is set to yes;

2) Protocol Setup should implement the cleaning solution; the solution should be 1% Tween in PBS; the contact time should be 35 seconds, the flush volume 400 μl, and the aspirate volume is 15%;

3) The arrays should print 55 samples in duplicate or 110 spots on a 16 Pad Fast Slide;

Upon Error, a retry should be attempted once before ignoring.

Printing:

1) Peptide Samples (2 mg/ml in H₂O) along with controls arrive in 96 well plates and only need to be properly positioned in the source holder;

After printing, all slides need to be properly labeled.

Repeat above to clean for next printing.

All references and patents cited herein are expressly incorporated herein in their entirety by reference.

We claim:

1. A set of informative epitopes for distinguishing between a plurality of classes for a biological sample, comprising at least one epitope set forth in any of Tables 1, 7-10 and FIGS. 2 and 3, wherein the autoantibody binding activity of each informative epitope is independently higher in a sample characteristic of one of the plurality of particular classes than in a sample characteristic of another one of the plurality of particular classes.

2. The set of informative epitopes according to claim 1, comprising at least two epitopes set forth in any of Tables 1, 7-10 and FIGS. 2 and 3.

3. The set of informative epitopes according to claim 1, comprising at least five epitopes set forth in any of Tables 1, 7-10 and FIGS. 2 and 3.

4. The set of informative epitopes according to claim 1, comprising at least 10 epitopes set forth in any of Tables 1, 7-10 and FIGS. 2 and 3.

5. The set of informative epitopes according to claim 1, comprising at least 15 epitopes set forth in any of Tables 1, 7-10 and FIGS. 2 and 3.

6. The set of informative epitopes according to claim 1, comprising at least 25 epitopes set forth in any of Tables 1, 7-10 and FIGS. 2 and 3.

7. The set of informative epitopes according to claim 1, comprising at least 50 epitopes set forth in any of Tables 1, 7-10 and FIGS. 2 and 3.

8. The set of informative epitopes according to any one of claims 1-7, wherein at least two informative epitopes correspond to distinct regions of a single protein.

9. The set of informative epitopes according to claim 8, wherein the at least two informative epitopes correspond to non-overlapping sequences within the single protein.

10. The set of informative epitopes according to any one of claims 1-9, wherein the set of informative epitopes is capable of distinguishing between a disease class and a non-disease class, wherein the disease class is cancer.

11. The set of informative epitopes according to claim 10, wherein the autoantibody binding activity of at least one informative epitope is higher in the non-disease class than in the disease class.

12. The set of informative epitopes according to claim 10, wherein the set of informative epitopes is capable of distinguishing tumor stages.

13. The set of informative epitopes according to claim 10, wherein the disease class is lung cancer.

14. The set of informative epitopes according to claim 13, comprising the 51 epitopes set forth in Table 2.

15. The set of informative epitopes according to claim 13, comprising the epitopes TRP-2/4, HAGHL-237, IQWD1-315, KIAA0373-1107, KIAA0373-1193, LOC401193-156, MSLN-186, NACA-261, NISCH-805, NISCH-1271, NISCH-1105, RBMS1-108, ROCK2-1296, SDCCAG3-255, SDCCAG8-815, TP53-171, UTP14A-818, UTP14A-182, ZNF292-3415, ZNF292-1612, ZNF292-3154, MELK-67, MELK-241, NFRKB-1575, AARS-1017, ACAT2-488, CTTNBP2-254, DDX5-190, DNAJA1-21, DNM1L-3, DRCTNNB1A-588, ELKS-241, GOLGA2-1061, IQWD1-575, LIMS1-182, LMNA-417, MKRN1-483, NAP1L3-145, RBM25-978, RBPSUH-350, RBPSUH-236, SDCCAG1-232, SR-A1-1126, and NY-ESO-1/2 set forth in Table 2.

16. The set of informative epitopes according to claim 13, comprising the epitopes IQWD1-315, KIAA0373-1107, NISCH-805, NISCH-1105, RBMS1-108, UTP14A-182, ZNF292-1612, NFRKB-1575, GOLGA2-1061, IQWD1-575, LMNA-417, NAP1L3-145, and RBM25-978 set forth in Table 2.

17. The set of informative epitopes according to claim 13, comprising the epitopes IQWD1-315, NISCH-1105, RBMS1-108, ZNF292-1612, CTTNBP2-254, DDX5-190, ELKS-241, RBPSUH-350, and RBPSUH-236 set forth in Table 2.

18. The set of informative epitopes according to claim 13, comprising the epitopes IQWD1-315, KIAA0373-1107, KIAA0373-1193, NISCH-805, NISCH-1105, RBMS1-108, ZNF292-1612, LMNA-417, and RBPSUH-236 set forth in Table 2.

19. The set of informative epitopes according to claim 13, comprising the 25 epitopes set forth in Table 11.

20. The set of informative epitopes according to claim 13, comprising the 28 epitopes set forth in FIG. 3.

21. The set of informative epitopes according to any one of claims 1-20, wherein the set of informative epitopes is capable of distinguishing between NSCLC and SCLC.

22. The set of informative epitopes according to claim 10, wherein the disease class is breast cancer.

23. The set of informative epitopes according to claim 22, comprising the 27 epitopes set forth in FIG. 2.

24. A method for diagnosing lung cancer, comprising detecting autoantibody binding activity in a patient sample using the set of informative epitopes according to any one of claims 1-21.

25. A method for diagnosing breast cancer, comprising detecting autoantibody binding activity in a patient sample using the set of informative epitopes according to any one of claims 1-12, 22 and 23.

26. A method for determining cancer prognosis, comprising detecting autoantibody binding activity in a cancer patient sample using the set of informative epitopes according to claim 12.

27. The method according to any one of claims 24-26, wherein the set of informative epitopes is present on an epitope microarray.

28. An epitope microarray, comprising the set of informative epitopes according to any one of claims 1-23.
